(19) United States
(12) Patent Application Publication
(10) Pub. No.: US 2024/0376143 A1
(43) Pub. Date: Nov. 14, 2024

(54) SELECTION INHIBITION OF DNA POLYMERASE BETA BY A COVALENT INHIBITOR

(71) Applicant: The Johns Hopkins University, Baltimore, MD (US)

(72) Inventors: Marc M. GREENBERG, Baltimore, MD (US); Shelby C. YUHAS, Baltimore, MD (US)

(21) Appl. No.: 18/560,583

(22) PCT Filed: May 17, 2022
(86) PCT No.: PCT/US2022/029573

§ 371 Date: Nov. 13, 2023

Related U.S. Application Data
(60) Provisional application No. 63/189,535, filed on May 17, 2021.

(51) Int. Cl. C07H 19/10; A61K 31/7072; A61P 35/00; C12N 9/99; G01N 21/64; G01N 33/68

(52) U.S. Cl. CPC C07H 19/10; A61K 31/7072; A61P 35/00; C12N 9/99; G01N 21/6428; G01N 33/68; G01N 2021/6432; G01N 2021/6439; G01N 2333/9126

(57) ABSTRACT

Methods and compounds are disclosed for irreversibly inhibiting a DNA polymerase, including DNA polymerase β (pol β). Also disclosed are methods for inducing a synthetic lethality in a breast cancer type 1 (BRCA1)-deficient cancer cell, the method comprising inhibiting DNA polymerase β by administering a presently disclosed pol β inhibitor.

### STATEMENT OF GOVERNMENTAL INTEREST

This invention was made with government support under grant GM131736 awarded by the National Institutes of Health. The government has certain rights in the invention.

### SEQUENCE LISTING

The text of the computer readable sequence listing filed herewith, titled “39414-601_SEQUENCE_LISTING_ST25”, created May 17, 2022, having a file size of 2,348 bytes, is hereby incorporated by reference in its entirety.

### BACKGROUND

DNA damage and repair have significant biological consequences on ageing and diseases. Zhao and Sumberaz, 2020; Nelson and Dizdaroglu, 2020; Thompson and Cortez, 2020; Lindahl, 2016. Consequently, the DNA damage response system is an increasingly popular inhibition target. A recent report documenting that defects in DNA repair explains why some cancer patients exhibit exceptional responses to chemotherapy, provides additional incentive for developing repair enzyme inhibitors. Wheeler et al., 2020. Poly(ADP-ribose) polymerase (PARP) inhibitors, of which four are FDA approved for treating BRCA1-deficient cancers, are leading the way. Farmer et al., 2005; Rottenberg et al., 2008; Lord and Ashworth, 2017. A variety of other enzymes involved in DNA repair, including glycosylases, Tahara et al., 2019; Tahara et al., 2018; Visnes et al., 2018; Donley et al., 2015; Huang et al., 2009, phosphodiesterases, Rai et al., 2012; Dorjsuren et al., 2012, and polymerases, Wojtaszek et al., 2019; Gowda et al., 2017; Strittmatter et al., 2011; Strittmatter et., 2013; Jaiswal et al., 2015; Arian et al., 2014; Paul et al., 2017; Paul et al., 2018; Goellner et al., 2012, also are attractive targets. DNA repair inhibitors can work in conjunction with damaging agents to kill cells. Alternatively, as in the case of the PARP inhibitors, some enzymes can be targeted to exploit a synthetic lethal relationship involving a second enzyme that is defective in a cell to selectively kill them.

### SUMMARY

In some aspects, the presently disclosed subject matter provides a compound of formula (I):

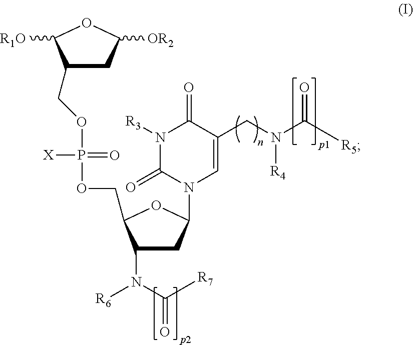

wherein:
n is an integer selected from 1, 2, 3, 4, 5, 6, 7, and 8;
X is selected from the group consisting of substituted or unsubstituted C₁-C₆ alkyl, O—S—, and —OR₈, wherein R₈ is substituted or unsubstituted C₁-C₆ alkyl;
p1 and p2 are each independently an integer selected from 0 or 1;
R₁ and R₂ are each independently selected from the group consisting of hydrogen and —C(═O)—R₉, wherein R₉ is selected from the group consisting of substituted or unsubstituted C₁-C₆ alkyl, heteroalkyl, cycloalkyl, cycloheteroalkyl, aryl, heteroaryl, arylalkyl, and heteroarylalkyl;
R₃, R₄, and R₆ are each independently H or C₁-C₆ alkyl;
R₅ and R₇ are each independently derived from a carboxylic acid-containing moiety selected from the group consisting of substituted or unsubstituted alkyl, heteroalkyl, cycloalkyl, cycloheteroalkyl, aryl, heteroaryl, arylalkyl, heteroarylalkyl, multicyclic aliphatic ring systems, multicyclic aromatic ring systems, fused aliphatic ring systems, fused aromatic ring systems, and combinations thereof; and
pharmaceutically acceptable salts thereof.

In certain aspects, R₁ and R₂ are each independently H or —(C═O)—CH₃.

In particular aspects, the compound of formula (I) is:

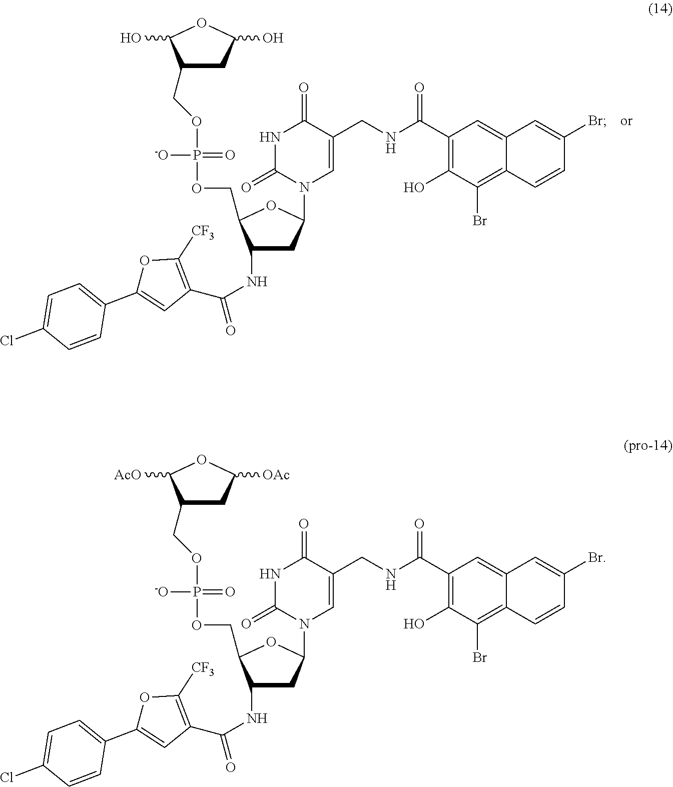

In other aspects, the presently disclosed subject matter provides a method for irreversibly inhibiting a DNA polymerase in a subject, the method comprising administering to the subject a therapeutically effective amount of a compound of Formula (I), or a pharmaceutically acceptable salt thereof.

In certain aspects, the DNA polymerase comprises a DNA repair enzyme. In particular aspects, the DNA repair enzyme is DNA polymerase β. In certain aspects, the compound of Formula (I) inhibits the lyase and the polymerase activities of the DNA polymerase.

In particular aspects, the subject has cancer. In more particular aspects, inhibiting the DNA polymerase treats, inhibits, delays, or prevents the spread of the cancer in the subject. In yet more particular aspects, the method further comprises treating, inhibiting, delaying, or preventing the spread of the cancer by inhibiting at least one cancer cell involved in one or more biological processes selected from the group consisting of cell migration, cell growth, cell adhesion, angiogenesis, cancer cell invasion, apoptosis, tumor formation, tumor progression, metastasis, degradation of the extracellular matrix, pericellular proteolysis, activation of plasminogen, and changes in the levels of an extracellular protease.

In certain aspects, the method further comprises administering to the subject a DNA damaging agent. In particular aspects, the DNA damaging agent is methyl methanesulfonate (MMS) or bleomycin.

In certain aspects, the DNA damaging agent is administered before or simultaneously with administration of the compound of Formula (I). In particular aspects, the compound of formula (I) and the DNA damaging agent have a synergistic effect in treating a cancer.

In other aspects, the presently disclosed subject matter provides a method for inhibiting a cancer cell, the method comprising contacting the cancer or noncancerous cell with a compound of Formula (I), or a pharmaceutically acceptable salt thereof, in an amount effective to irreversibly inhibit a DNA polymerase.

In yet other aspects, the presently disclosed subject matter provides a method for inducing a synthetic lethality in abreast cancer type 1 (BRCA1)-deficient cancer cell, the method comprising inhibiting DNA polymerase β by administering a compound of Formula (I) to the cell.

In even yet other aspects, the presently disclosed subject matter provides a method for identifying one or more DNA polymerase inhibitors, the method comprising: (a) contacting one or more candidate polymerase inhibitors with a DNA polymerase to form an inhibitor/polymerase solution; (b) contacting the inhibitor/polymerase solution with a DNA template and deoxynucleotide triphosphate(s) (dNTP), and (c) measuring polymerization using a fluorescently labeled DNA primer or a reagent that selectively binds to duplex DNA and fluoresces upon binding.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B demonstrate inhibition of DNA polymerase β by 14. (FIG. 1A) Ritz-Wilson plot of irreversible inhibition by 14. (FIG. 1B) IC₅₀ of 14 as a function of preincubation time. Data are the ave.±std. dev. of three replicates;

FIG. 2A and FIG. 2B show the effect of dialysis and pH on inactivation by 14. (FIG. 2A) Pol β strand displacement activity before and after dialysis with and without 14 (750 nM, 20 min preincubation). (FIG. 2B) pH Effect on inactivation by 14 (500 nM, 20 min) before and after dialysis. *Preincubation was carried out at pH 7.4, followed by dialysis at pH 8.0. Data are the ave.±std. dev. of three replicates;

FIG. 3A and FIG. 3B illustrate covalent modification of Pol β by 14. (FIG. 3A) Modified peptides observed in trypsin digest of Pol β preincubated with 14 (300 nM, 30 min). (FIG. 3B) Pol β crystal structure with modified residues (cyan spheres) (PDB: 1PBX);

FIG. 4 shows the effect of inhibitor 14 on DNA (0.25 nM) binding by Pol β. Data are the ave.±std. dev. of three replicates;

FIG. 5 shows inhibition of DNA polymerases by 14. Data are the ave.±std. dev. of three replicates;

FIG. 6A and FIG. 6B demonstrate treatment of mouse embryonic fibroblasts with MMS and pro-14. (FIG. 6A) MMS cytotoxicity with or without pro-14 in the presence or absence of Pol b. (FIG. B) Effect of greater pro-14 concentration. Data are the ave.±std. dev. of three replicates;

FIG. 7 shows the synergistic effect of DNA damaging agents with pro-14 in HeLa cells. Data are the ave.±std. dev. of three replicates;

FIG. 8A and FIG. 8B show (FIG. 8A) fluorescence-based strand displacement assay. (FIG. 8B) First-generation hit compounds from initial screen;

FIG. 9A and FIG. 9B demonstrate the inhibitory activity of first-generation inhibitor 7. (FIG. 9A) Pol β strand displacement inhibition following 20 min preincubation with 7 at various concentrations (listed). (FIG. 9B) Effect of preincubation time on inhibitory activity of 7 at 15 μM;

FIG. 10 shows the IC₅₀ value of 14 under exact conditions used to evaluate 2;

FIG. 11A and FIG. 11B shows the effect of pro-14 on MMS cytotoxicity in MEFs containing or lacking Pol λ (FIG. 11A) The effect of pro-14 on MMS cytotoxicity (0, 0.1, 0.2, 0.5, 1.0, 1.5 mM) in Pol λ WT and Pol λ−/− MEFs. (FIG. 11B) The effect of pro-14 on MMS cytotoxicity (0, 0.1, 0.2, 0.5, 1.0, 1.5 mM) in Pol λ WT and Pol β−/λ− MEFs;

FIG. 12 shows the effect of greater concentrations of pro-14 (0, 5, 15, 25 μM) on MMS cytotoxicity (0, 0.1, 0.2, 0.5, 1.0, 1.5 mM) in MEFs lacking Pol β and/or Pol λ.

FIG. 13 shows synthetic lethal interactions in isogenic ovarian cancer cells that are either proficient (UWB1.289+BRCA1) or deficient (UWB1.289) in homologous recombination. Synthetic lethality between Pol β and BRCA1 established using siRNA or pro-14. siNT=non-targeting siRNA; siPol β=siRNA for Pol β. Treatment time 2 h;

FIG. 14A, FIG. 14B, FIG. 14C, and FIG. 14D show synthetic lethal interactions between Pol β or PARP1 and BRCA1 in isogenic ovarian cancer cells (UWB1.289; UWB1.289+BRCA1). FIG. 14A) Synthetic lethality between Pol β and BRCA1 established using pro-14. Treatment time 1 h. FIG. 14B) Synthetic lethality between PARP1 and BRCA1 established using Olaparib. Treatment time 1 h. FIG. 14C) Cytotoxicity of pro-14 as a function of treatment time. FIG. 14D) Cytotoxicity of Olaparib as a function of treatment time;

FIG. 15A and FIG. 15B demonstrate cytotoxicity to mouse embryonic fibroblasts of (FIG. 15A) Pro-14. (FIG. 15B) Olaparib;

FIG. 16A and FIG. 16B show synthetic lethal interactions in breast cancer cells that are homologous recombination proficient (MCF-7) or deficient (MDAMB-436) due to BRCA1 deletion. (FIG. 16A) Synthetic lethality between Pol β and BRCA1 established using pro-14. (FIG. 16B) Synthetic lethality between PARP1 and BRCA1 established using Olaparib. Treatment time 1 h; and

FIG. 17A and FIG. 17B show the Western blot analysis of Pol β knock down in UWB1.289 cells. (FIG. 17A) Western blot and (FIG. 17B) Relative levels of Pol β expression in transfected cells. Data are avg±std. dev (three replicates).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Extra- and Intracellular Selective, Covalent Inactivation of DNA Polymerase Beta

In some embodiments, the presently disclosed subject matter provides compounds exhibiting unprecedented selectivity for the target, DNA polymerase β. In certain embodiments, the presently disclosed subject matter characterizes the mechanism of action and establishes the preference of representative compounds for DNA polymerase β versus four other polymerases. In further embodiments, the presently disclosed subject matter demonstrates that a corresponding pro-inhibitor acts on the target selectively in cells.

Accordingly, in some embodiments, the presently disclosed subject matter provides a compound of formula (I):

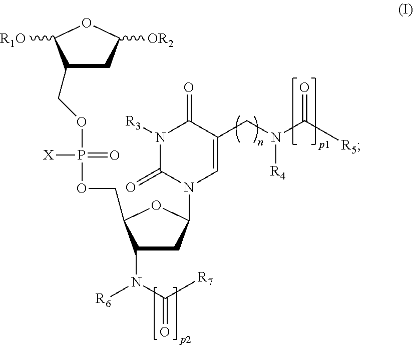

wherein:
n is an integer selected from 1, 2, 3, 4, 5, 6, 7, and 8;
X is selected from the group consisting of substituted or unsubstituted C₁-C₆ alkyl, O—, S—, and —OR₈, wherein R₈ is substituted or unsubstituted C₁-C₆ alkyl;
p1 and p2 are each independently an integer selected from 0 or 1;
R₁ and R₂ are each independently selected from the group consisting of hydrogen and —C(═O)—R₉, wherein R₉ is selected from the group consisting of substituted or unsubstituted C₁-C₆ alkyl, heteroalkyl, cycloalkyl, cycloheteroalkyl, aryl, heteroaryl, arylalkyl, and heteroarylalkyl;
R₃, R₄, and R₆ are each independently H or C₁-C₆ alkyl;
R₅ and R₇ are each independently derived from a carboxylic acid-containing moiety selected from the group consisting of substituted or unsubstituted alkyl, heteroalkyl, cycloalkyl, cycloheteroalkyl, aryl, heteroaryl, arylalkyl, heteroarylalkyl, multicyclic aliphatic ring systems, multicyclic aromatic ring systems, fused aliphatic ring systems, fused aromatic ring systems, and combinations thereof; and
pharmaceutically acceptable salts thereof.

In certain embodiments, X is O—. In other embodiments, X is —O—CH₂CH₂—C≡N.

In certain embodiments, R₁ and R₂ are each independently H or —(C═O)—CH₃.

In particular embodiments, R₅ and R₇ are each independently selected from the group consisting of:

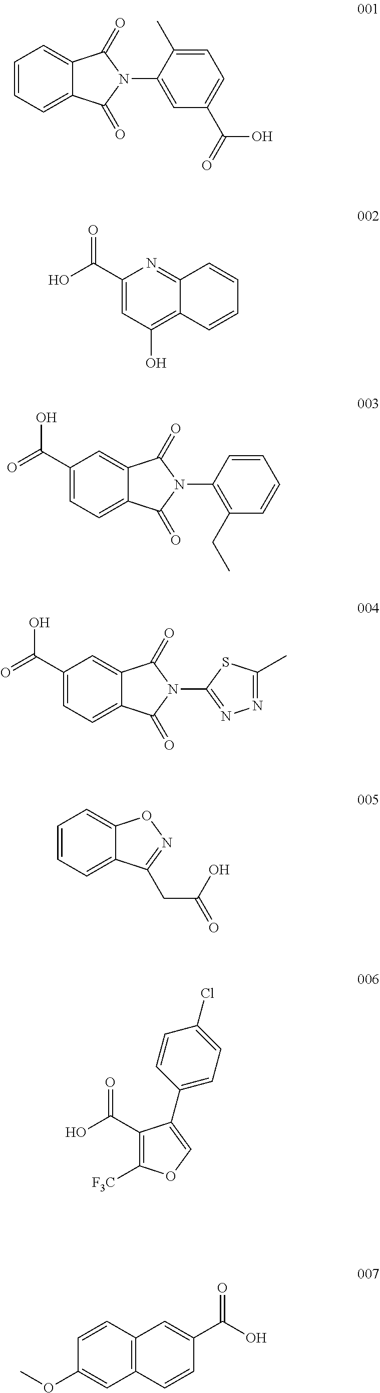

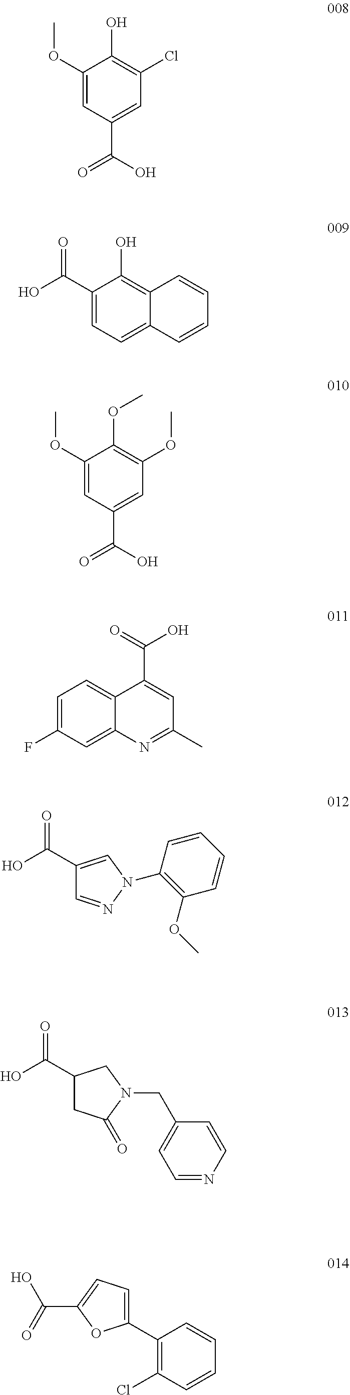

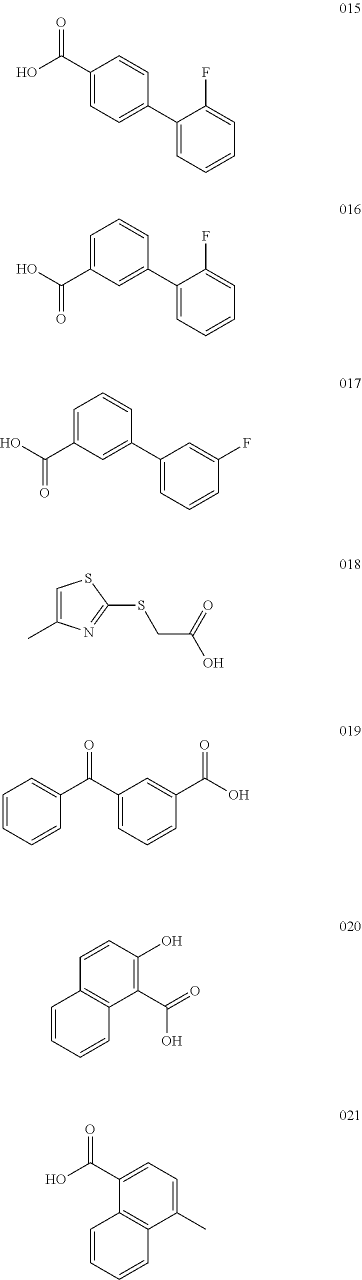

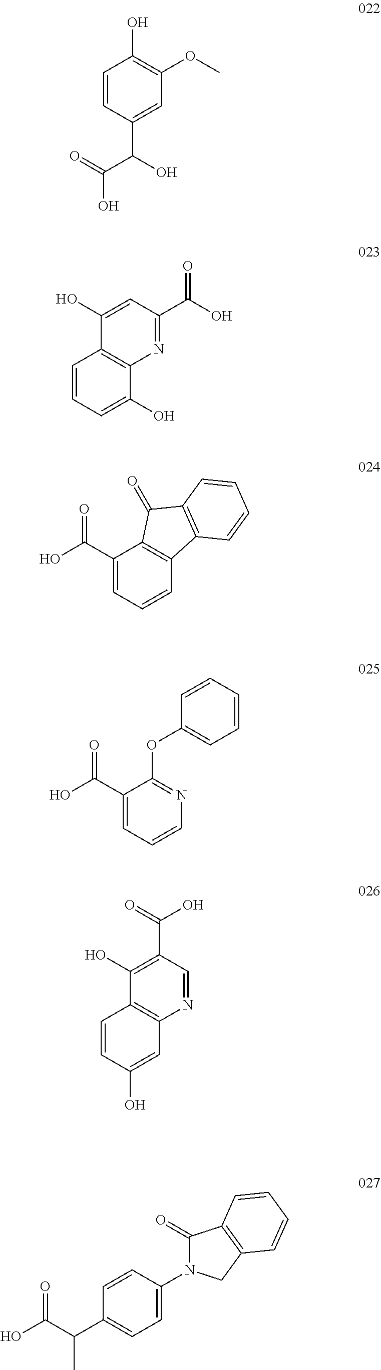 [SMILES: COc1cc(C(O)C(=O)O)ccc1O ; O=C(O)c1cc(O)c2cccc(O)c2n1 ; O=C(O)c1cccc2c1C(=O)c1ccccc1-2 ; O=C(O)c1cccnc1Oc1ccccc1 ; O=C(O)c1cnc2cc(O)ccc2c1O ; CC(C(=O)O)c1ccc(N2Cc3ccccc3C2=O)cc1]

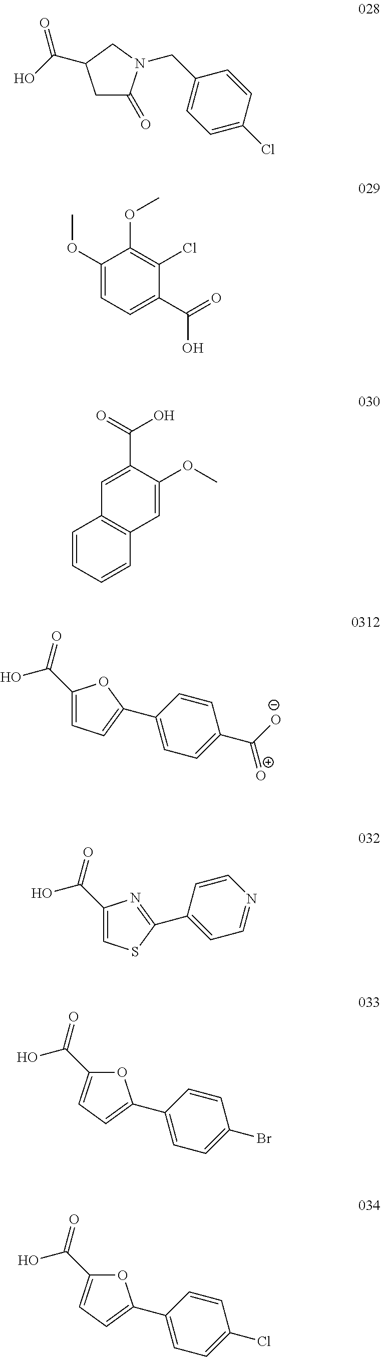

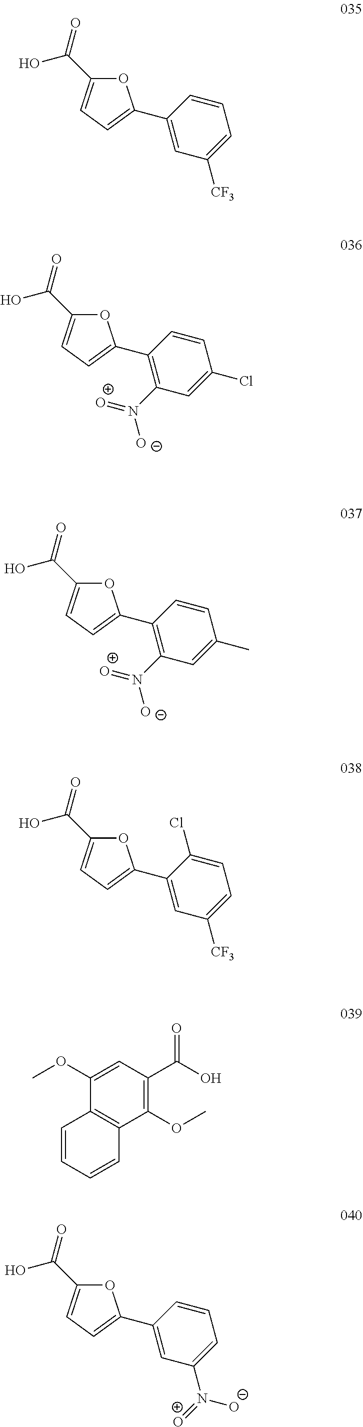

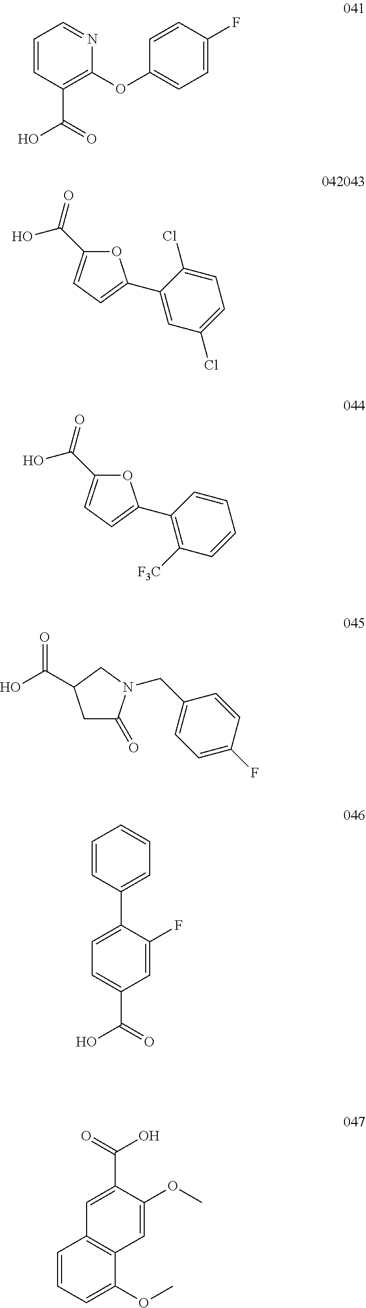 [SMILES: O=C(O)c1cccnc1Oc1ccc(F)cc1 ; O=C(O)c1ccc(-c2cc(Cl)ccc2Cl)o1 ; O=C(O)c1ccc(-c2ccccc2C(F)(F)F)o1 ; O=C(O)C1CC(=O)N(Cc2ccc(F)cc2)C1 ; O=C(O)c1ccc(-c2ccccc2)c(F)c1 ; COc1cc2c(OC)cccc2cc1C(=O)O]

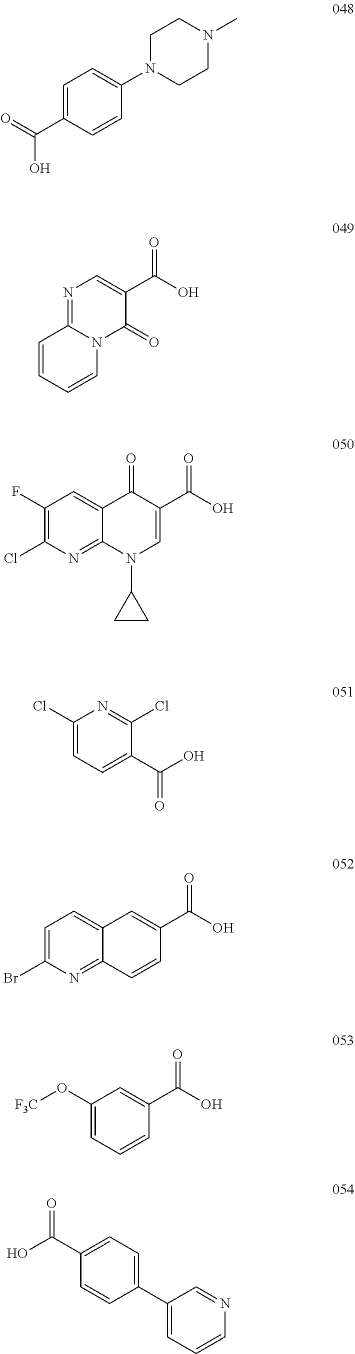

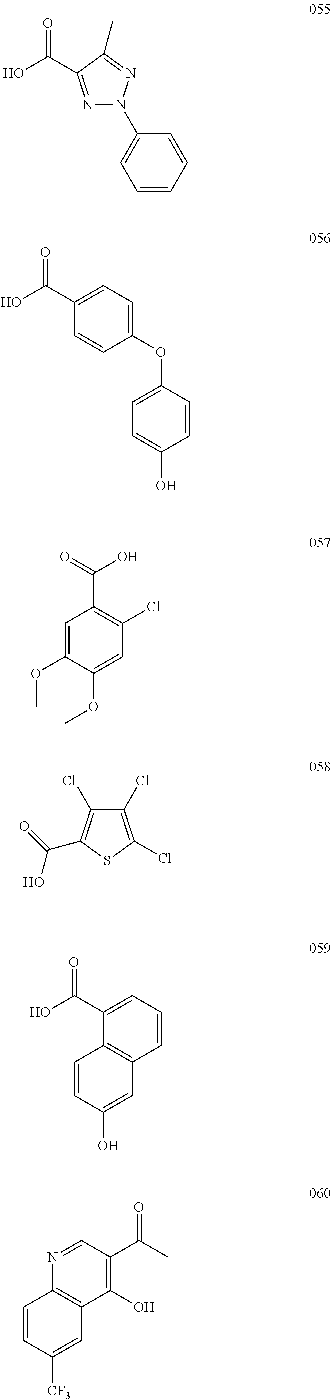 [SMILES: Cc1nn(-c2ccccc2)nc1C(=O)O ; O=C(O)c1ccc(Oc2ccc(O)cc2)cc1 ; COc1cc(Cl)c(C(=O)O)cc1OC ; O=C(O)c1sc(Cl)c(Cl)c1Cl ; O=C(O)c1cccc2cc(O)ccc12 ; CC(=O)c1cnc2ccc(C(F)(F)F)cc2c1O]

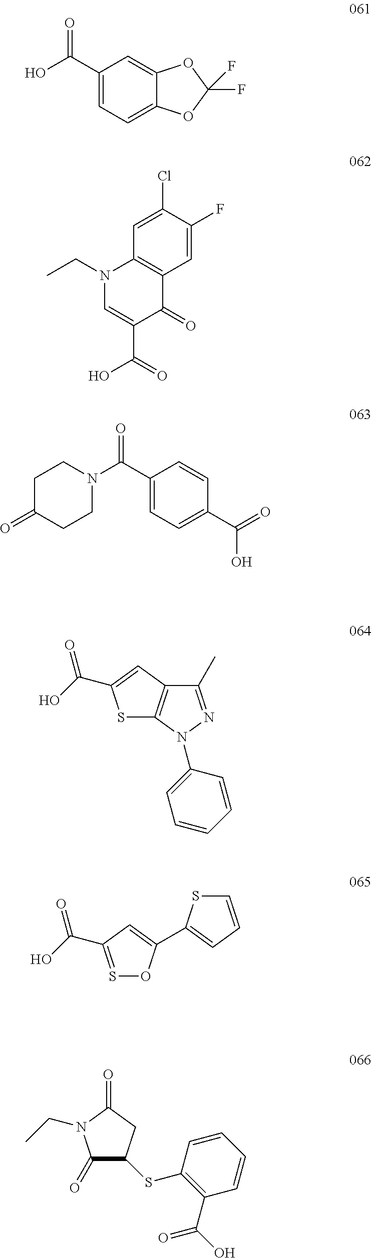

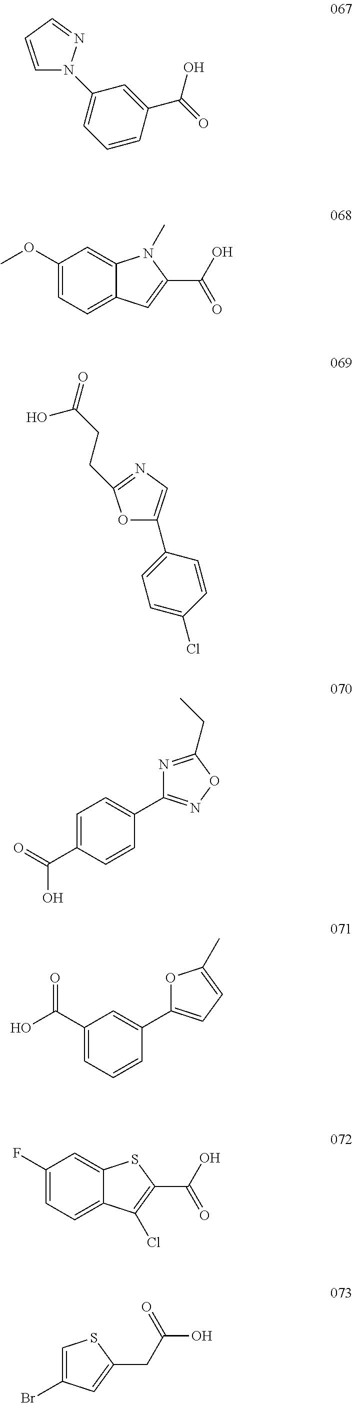

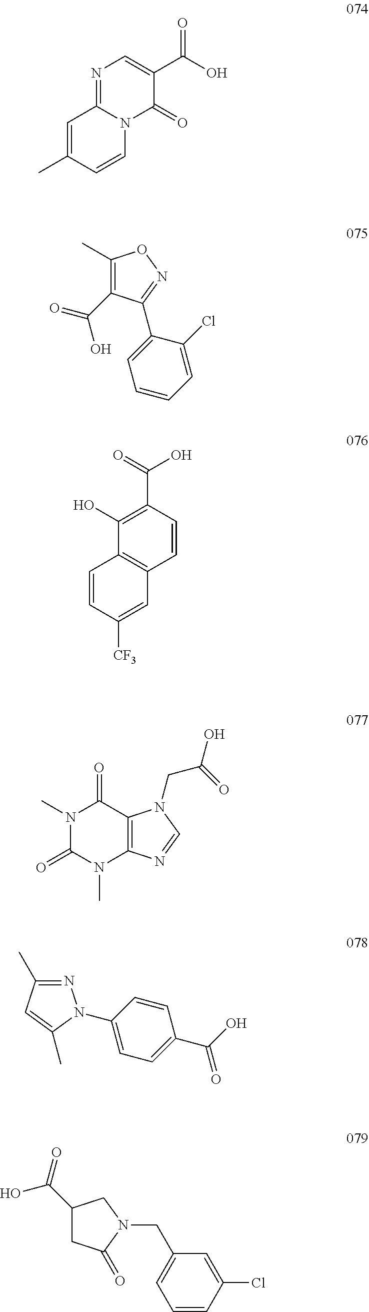 [SMILES: Cc1ccn2c(=O)c(C(=O)O)cnc2c1 ; Cc1onc(-c2ccccc2Cl)c1C(=O)O ; O=C(O)c1ccc2cc(C(F)(F)F)ccc2c1O ; Cn1c(=O)c2c(ncn2CC(=O)O)n(C)c1=O ; Cc1cc(C)n(-c2ccc(C(=O)O)cc2)n1 ; O=C(O)C1CC(=O)N(Cc2cccc(Cl)c2)C1]

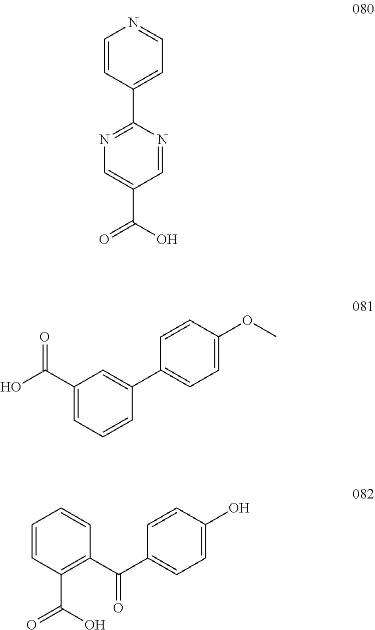 [SMILES: O=C(O)c1cnc(-c2ccncc2)nc1 ; COc1ccc(-c2cccc(C(=O)O)c2)cc1 ; O=C(O)c1ccccc1C(=O)c1ccc(O)cc1]

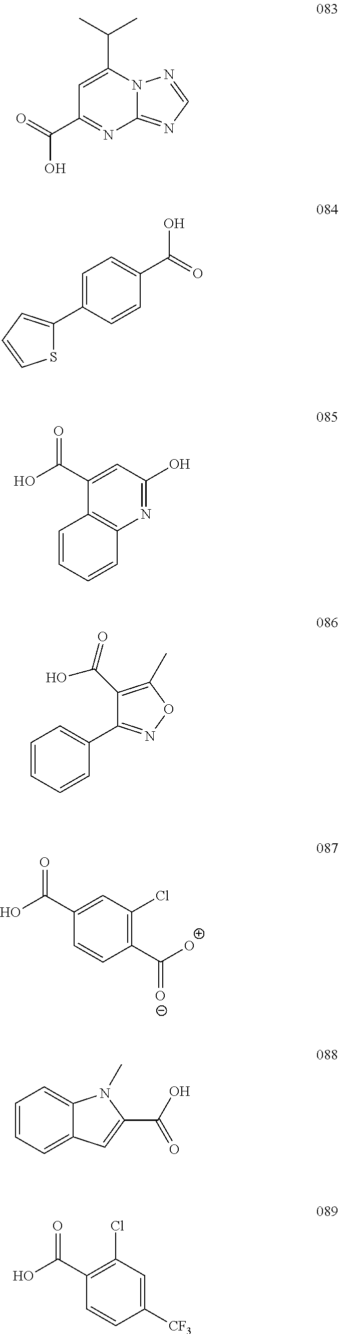

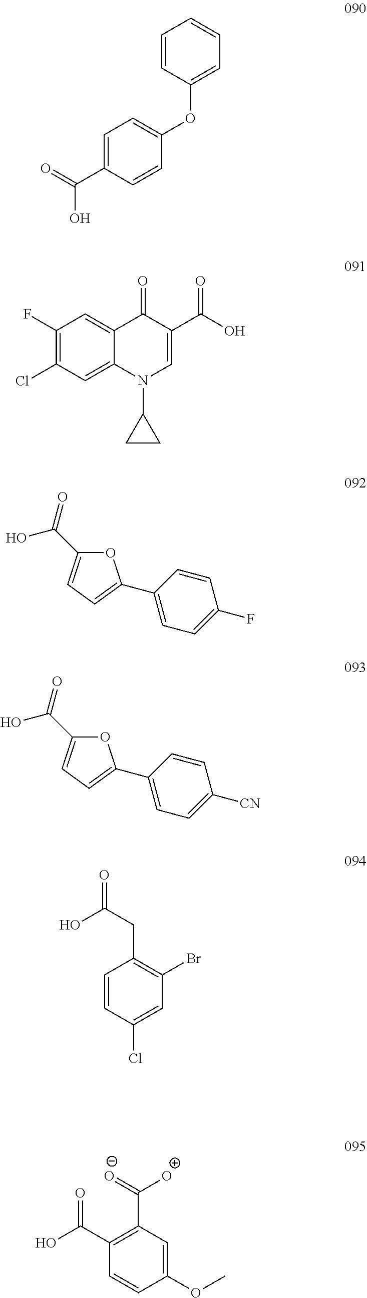

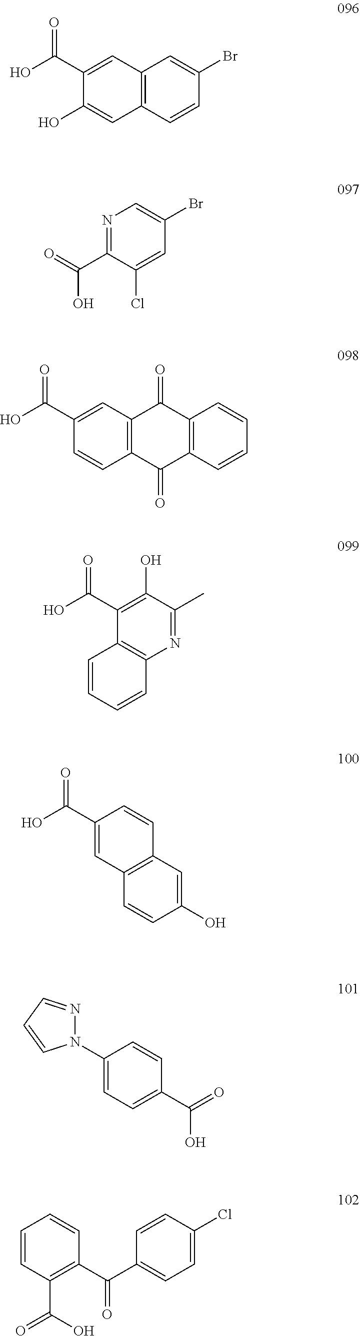

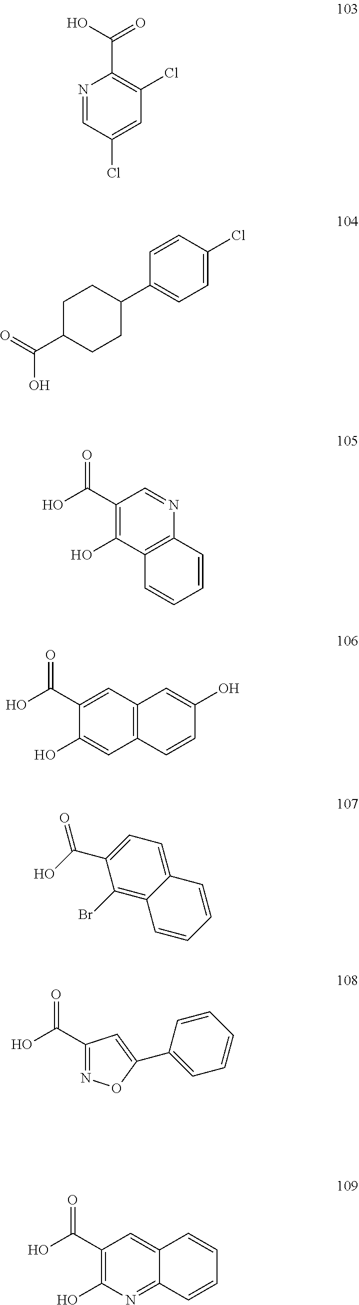

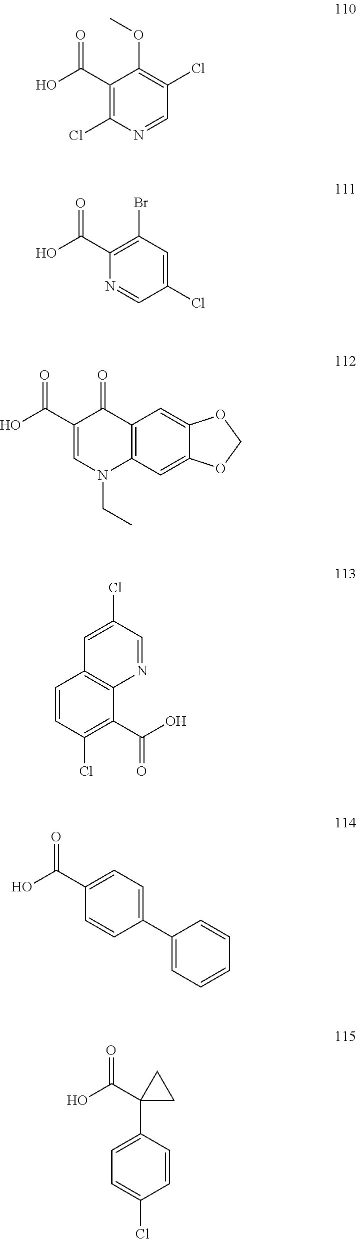 [SMILES: COc1c(Cl)cnc(Cl)c1C(=O)O ; O=C(O)c1ncc(Cl)cc1Br ; CCn1cc(C(=O)O)c(=O)c2cc3c(cc21)OCO3 ; O=C(O)c1c(Cl)ccc2cc(Cl)cnc12 ; O=C(O)c1ccc(-c2ccccc2)cc1 ; O=C(O)C1(c2ccc(Cl)cc2)CC1]

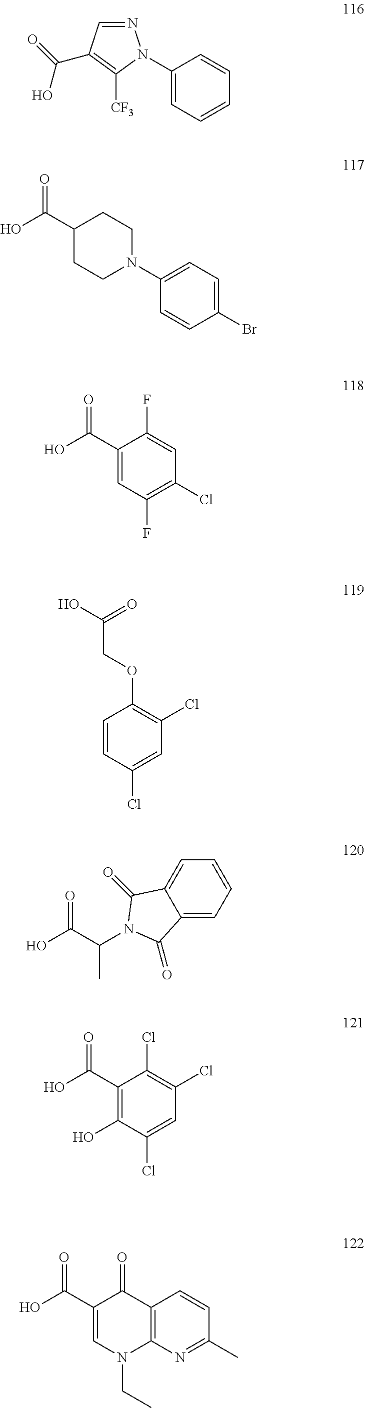

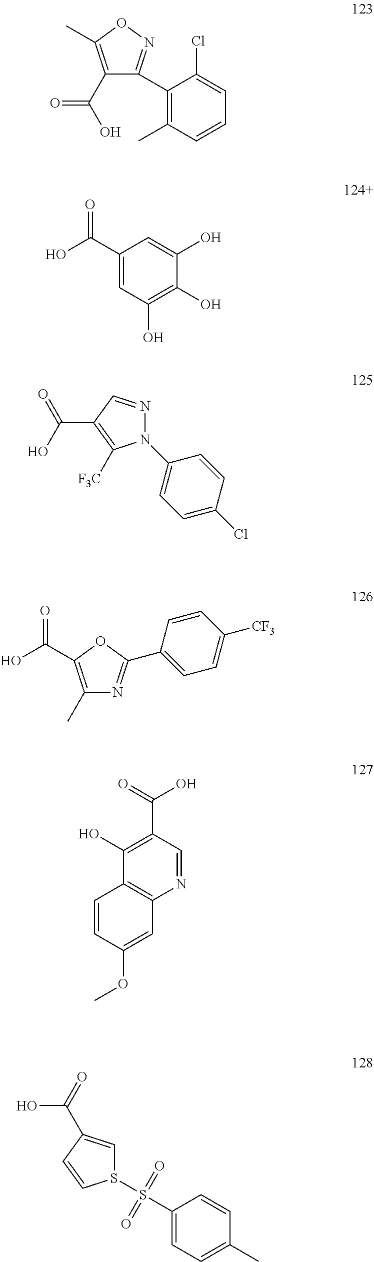

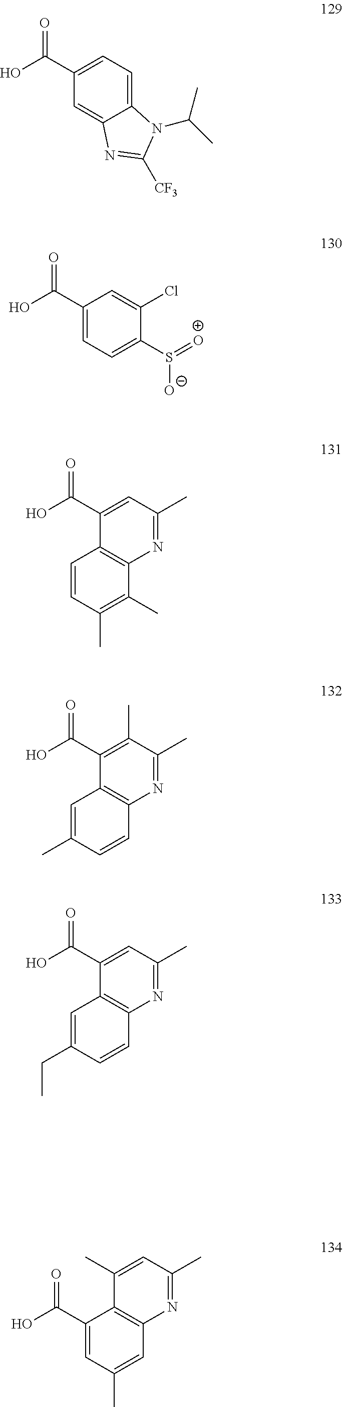 [SMILES: CC(C)n1c(C(F)(F)F)nc2cc(C(=O)O)ccc21 ; O=C(O)c1ccc(S([O-])=[OH+])c(Cl)c1 ; Cc1cc(C(=O)O)c2ccc(C)c(C)c2n1 ; Cc1ccc2nc(C)c(C)c(C(=O)O)c2c1 ; CCc1ccc2nc(C)cc(C(=O)O)c2c1 ; Cc1cc(C(=O)O)c2c(C)cc(C)nc2c1]

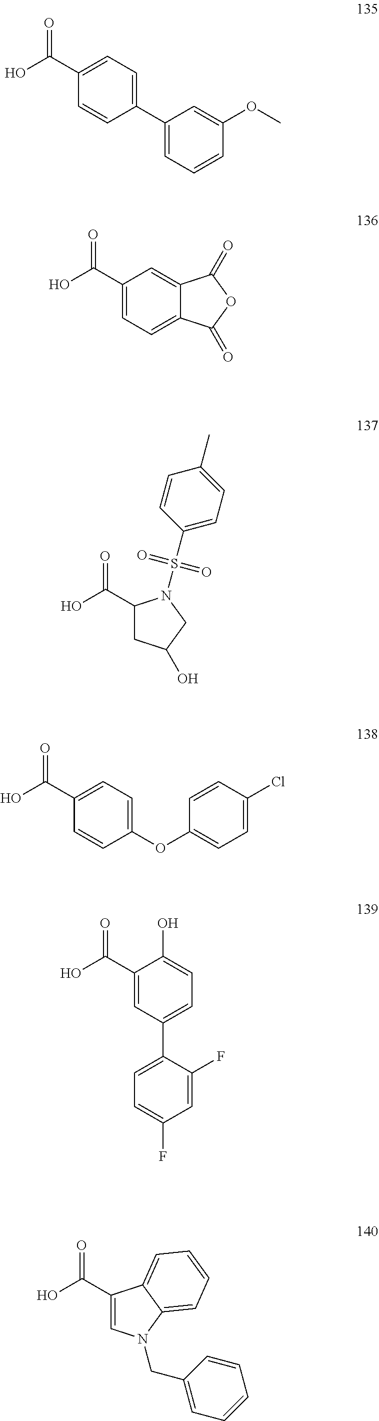 [SMILES: COc1cccc(-c2ccc(C(=O)O)cc2)c1 ; O=C(O)c1ccc2c(c1)C(=O)OC2=O ; Cc1ccc(S(=O)(=O)N2CC(O)CC2C(=O)O)cc1 ; O=C(O)c1ccc(Oc2ccc(Cl)cc2)cc1 ; O=C(O)c1cc(-c2ccc(F)cc2F)ccc1O ; O=C(O)c1cn(Cc2ccccc2)c2ccccc12]

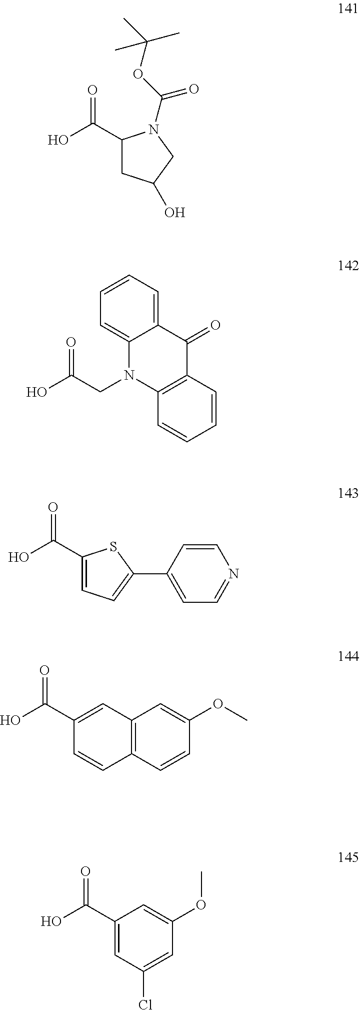 [SMILES: CC(C)(C)OC(=O)N1CC(O)CC1C(=O)O ; O=C(O)Cn1c2ccccc2c(=O)c2ccccc21 ; O=C(O)c1ccc(-c2ccncc2)s1 ; COc1ccc2ccc(C(=O)O)cc2c1 ; COc1cc(Cl)cc(C(=O)O)c1]

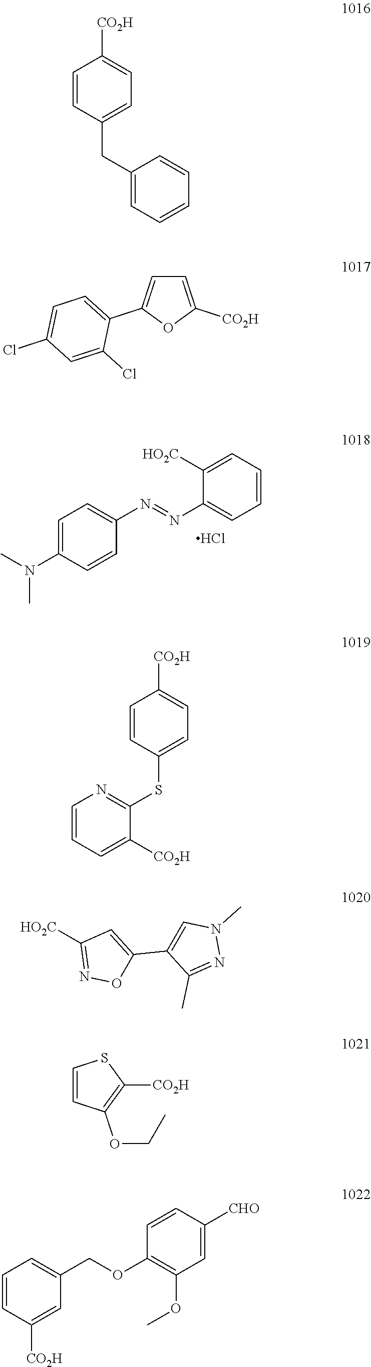

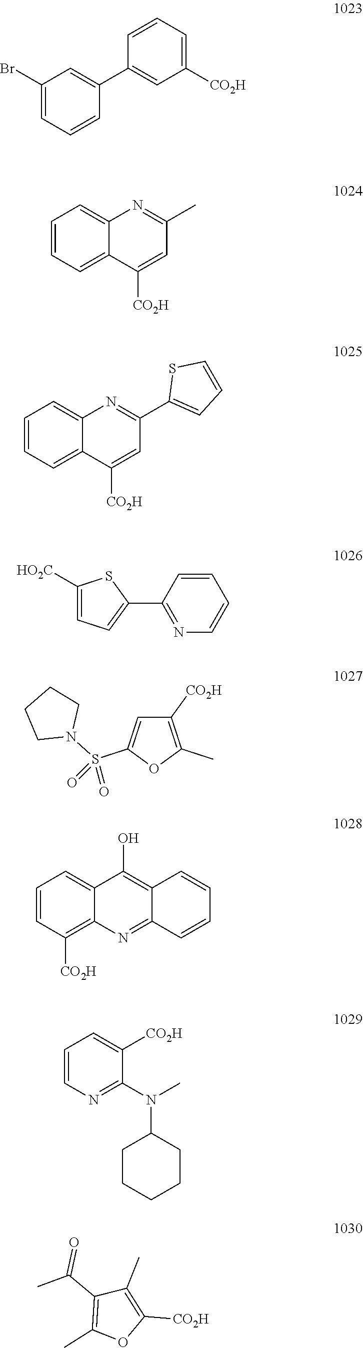

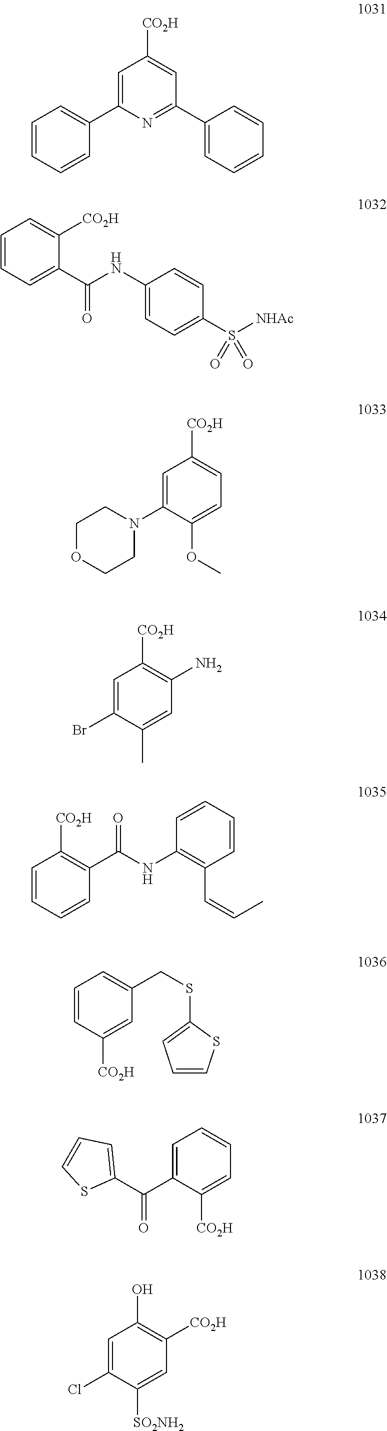

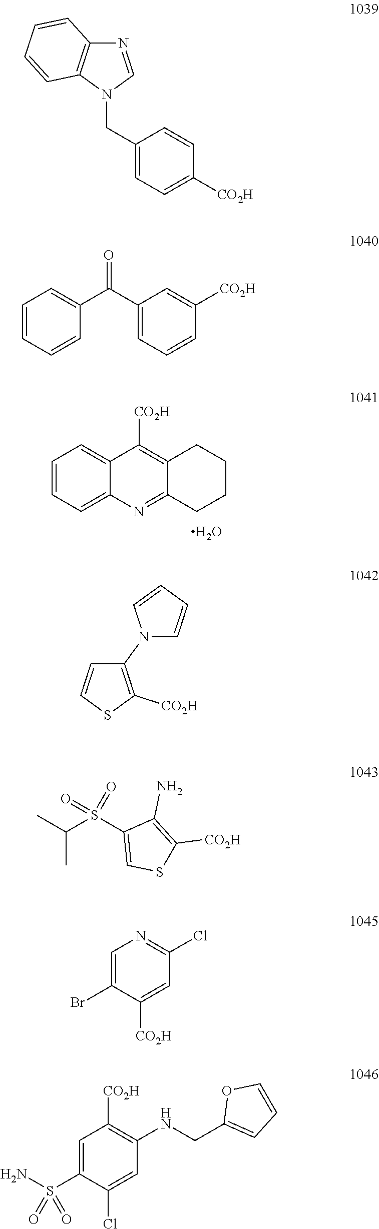

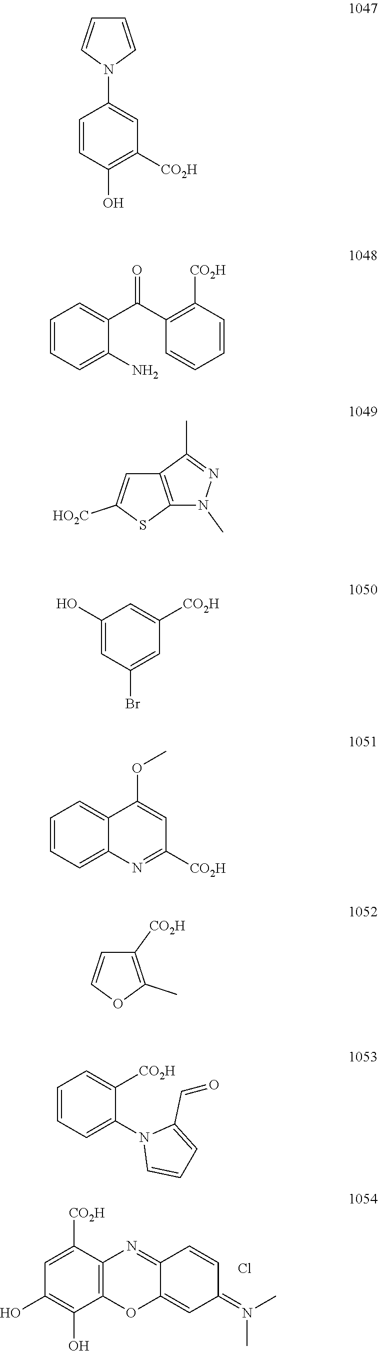

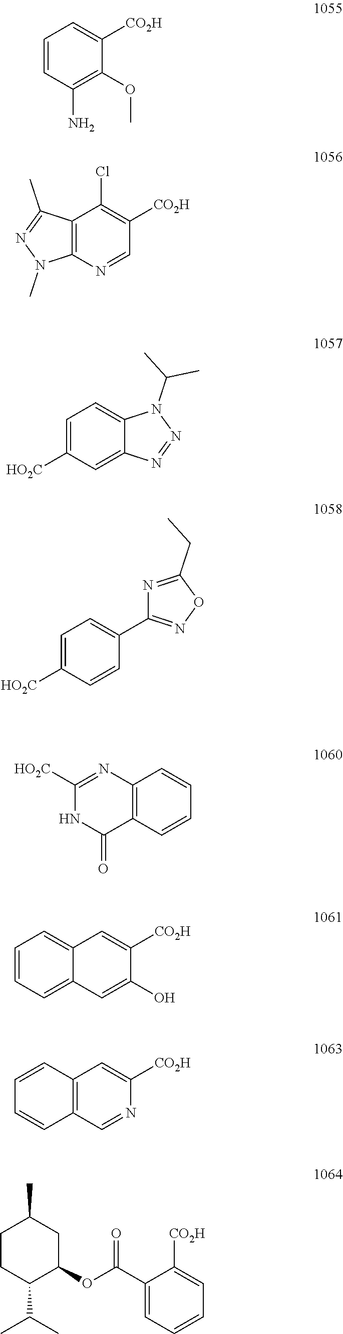

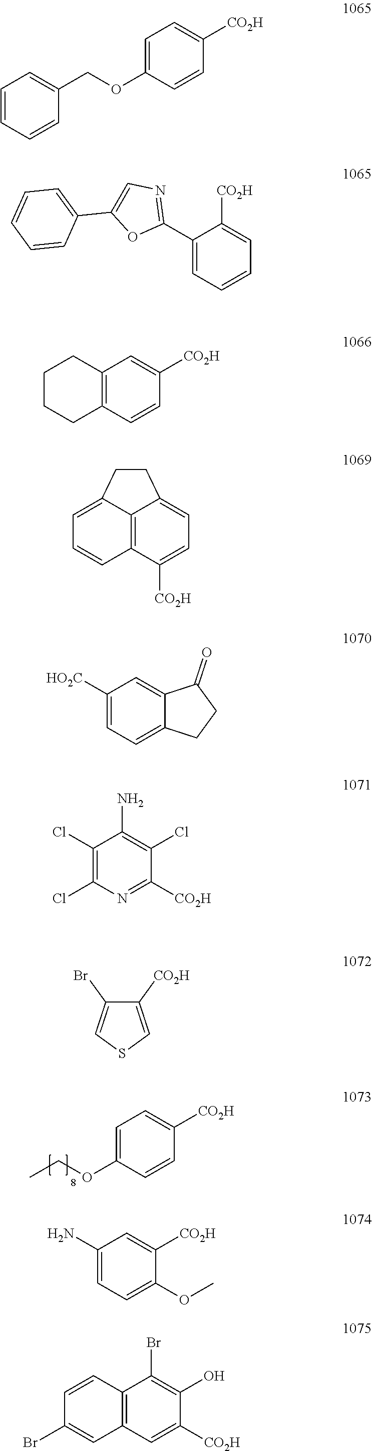

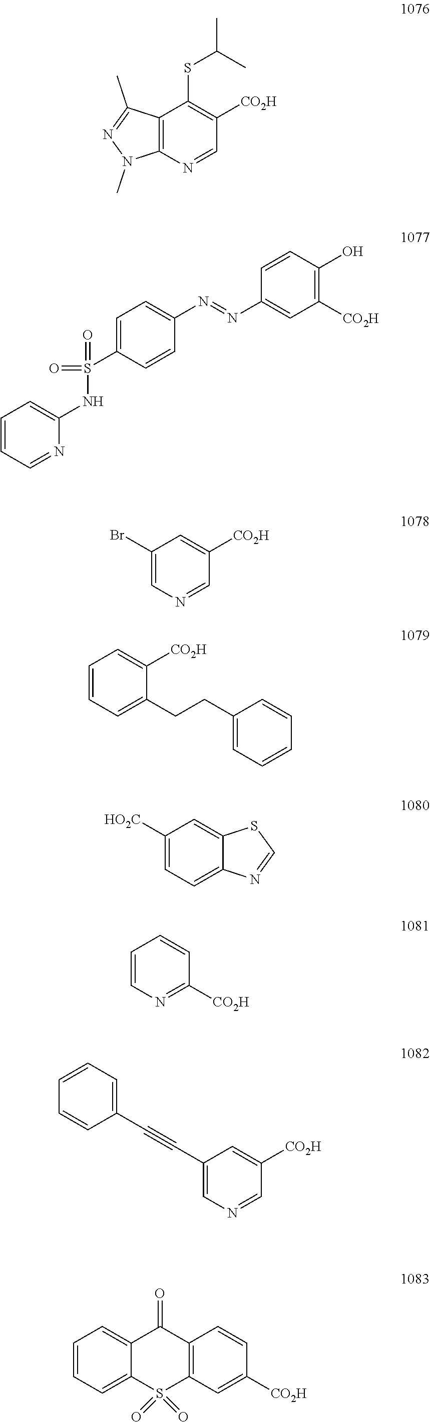

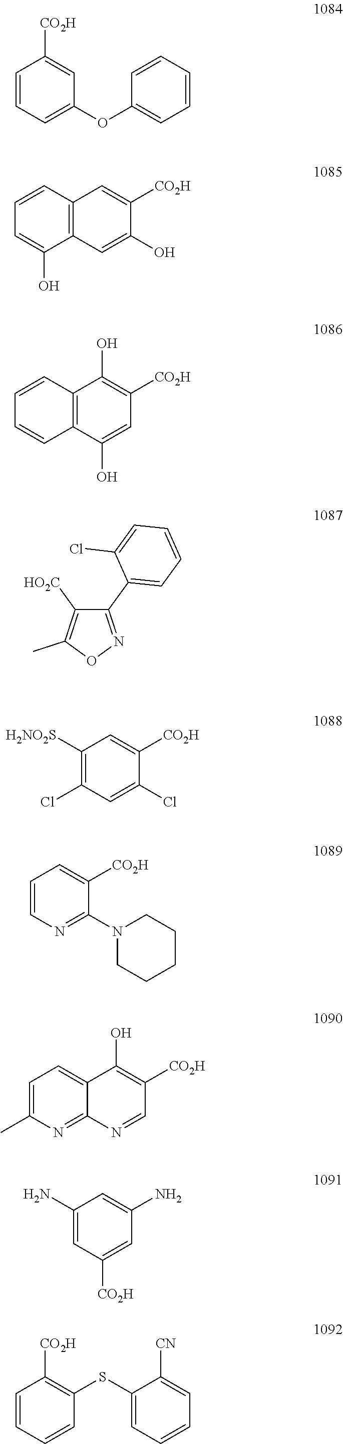

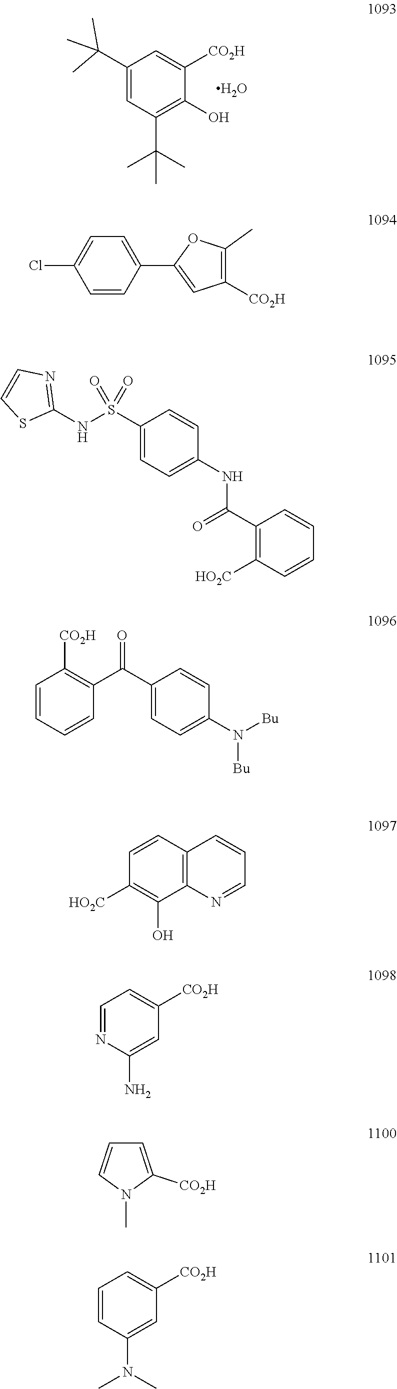

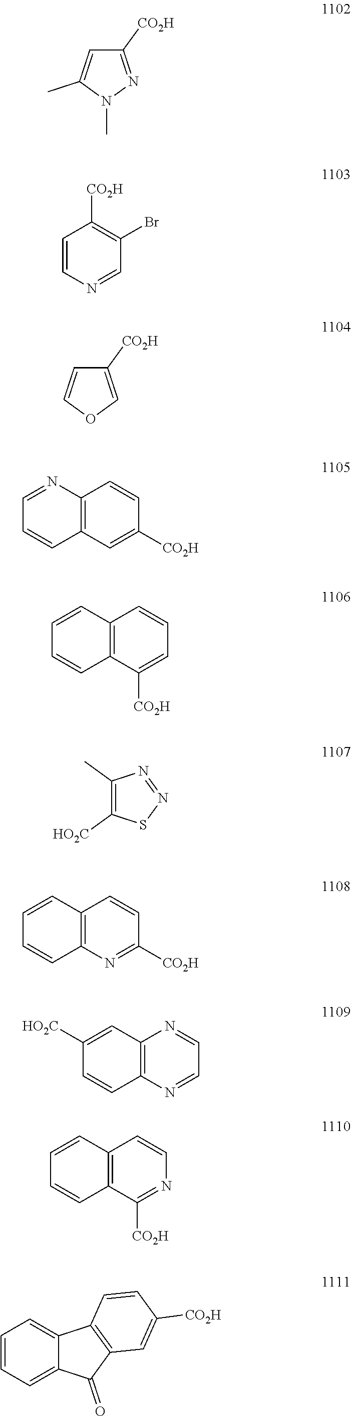

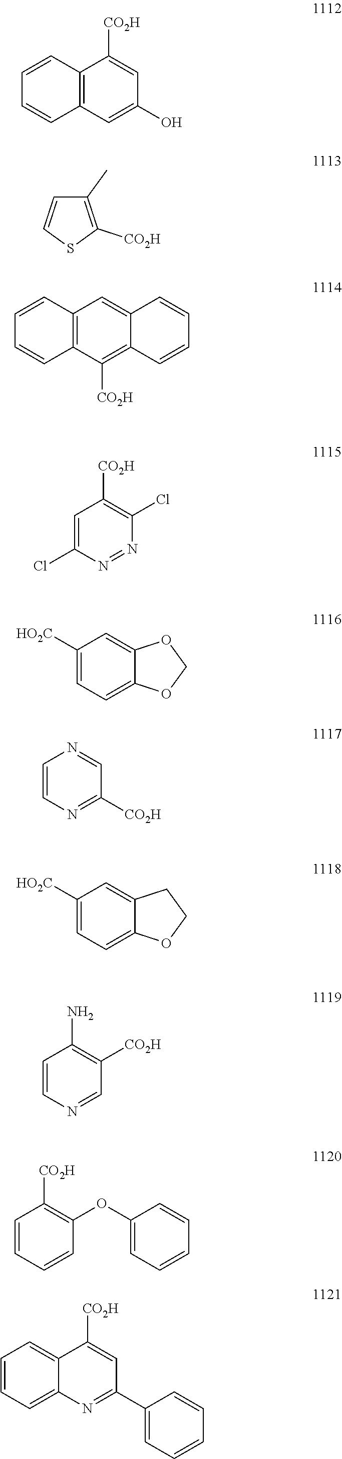

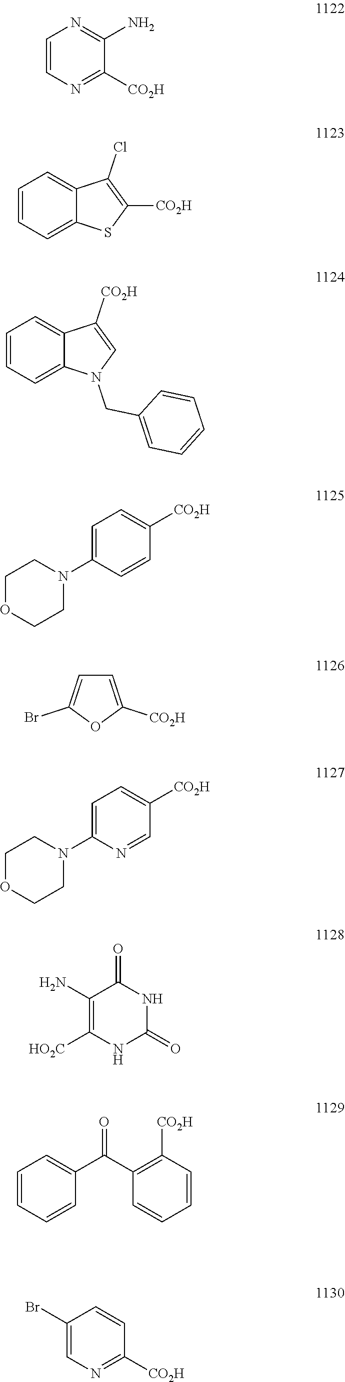

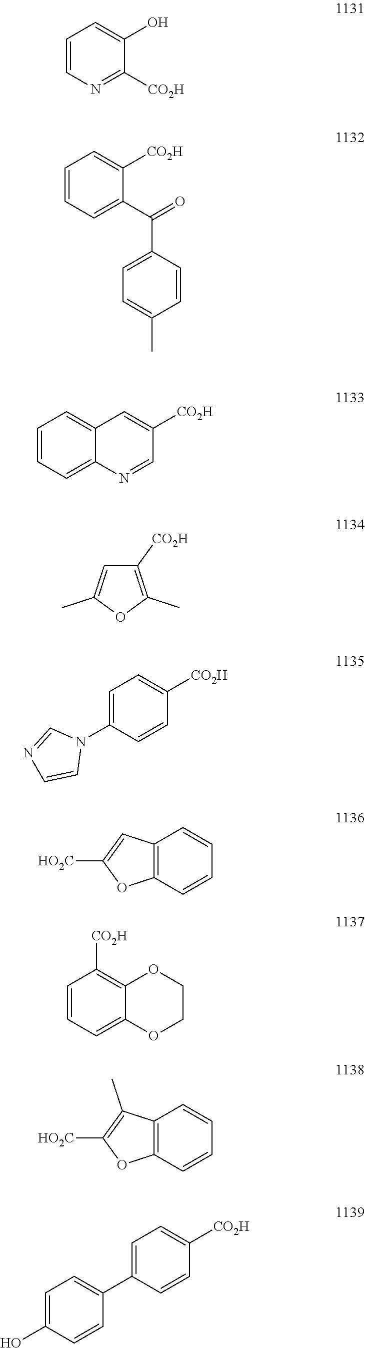

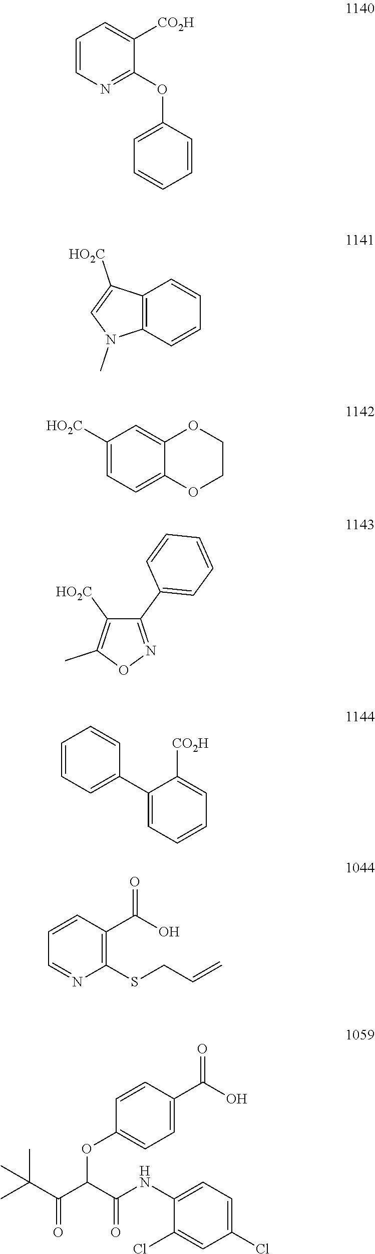

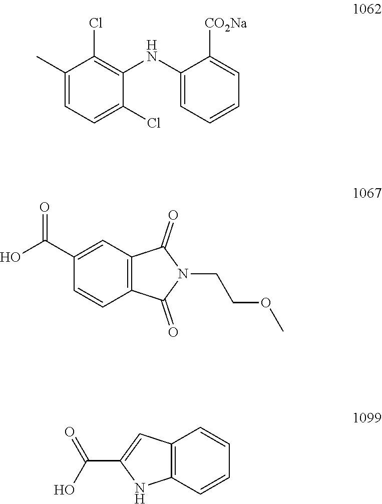

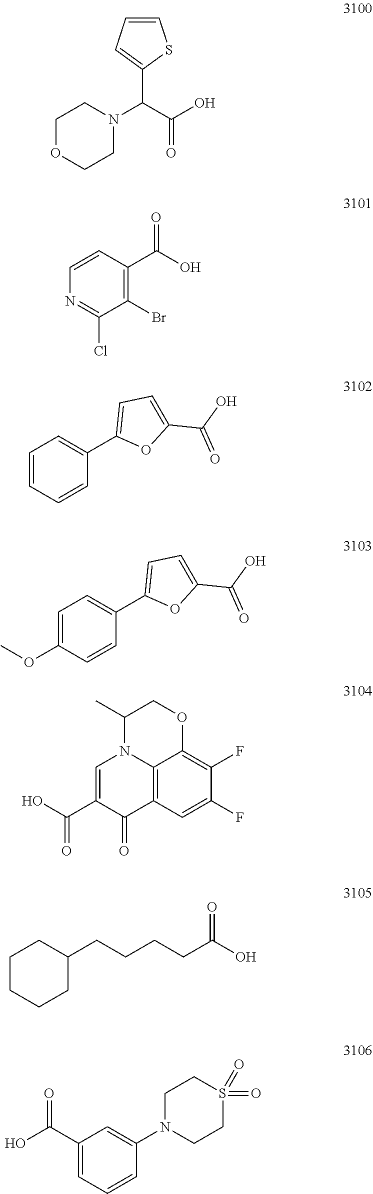

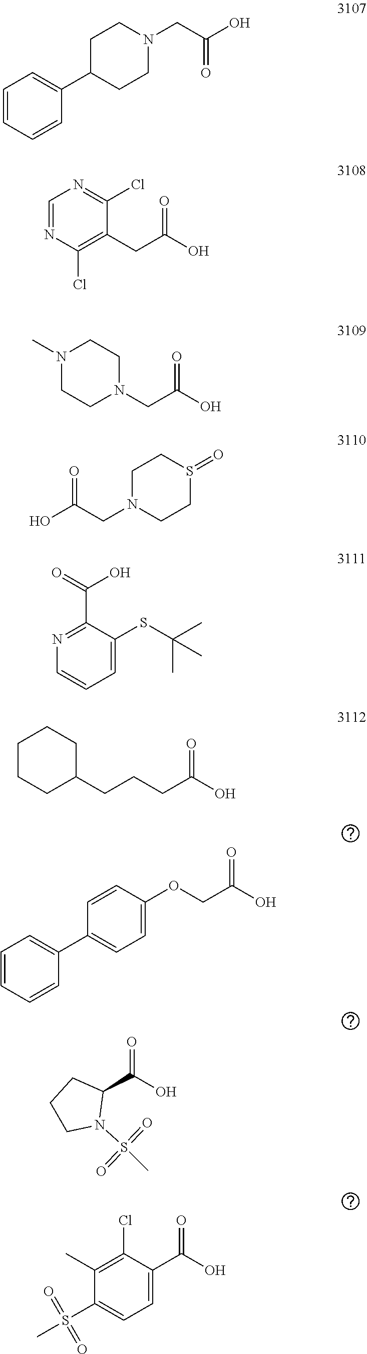

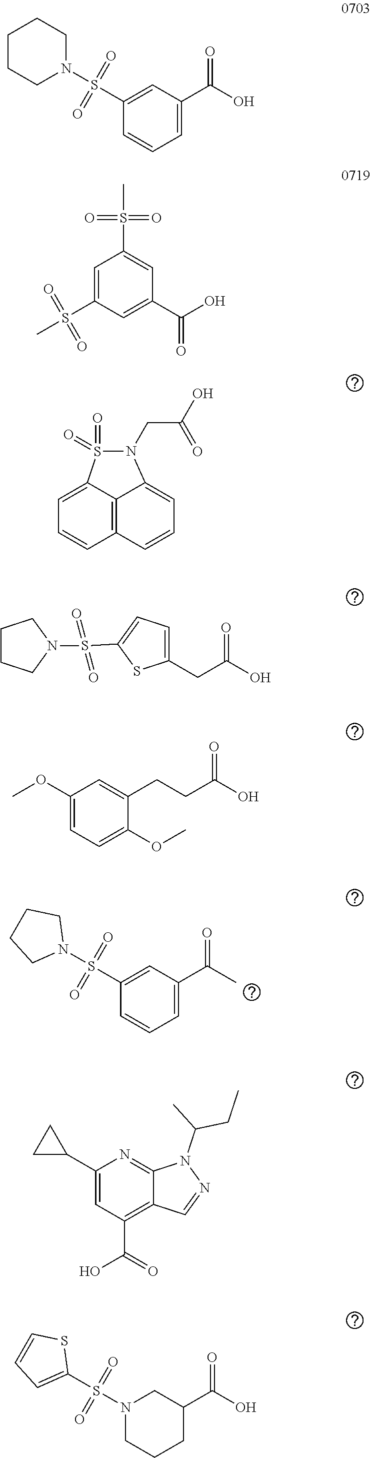

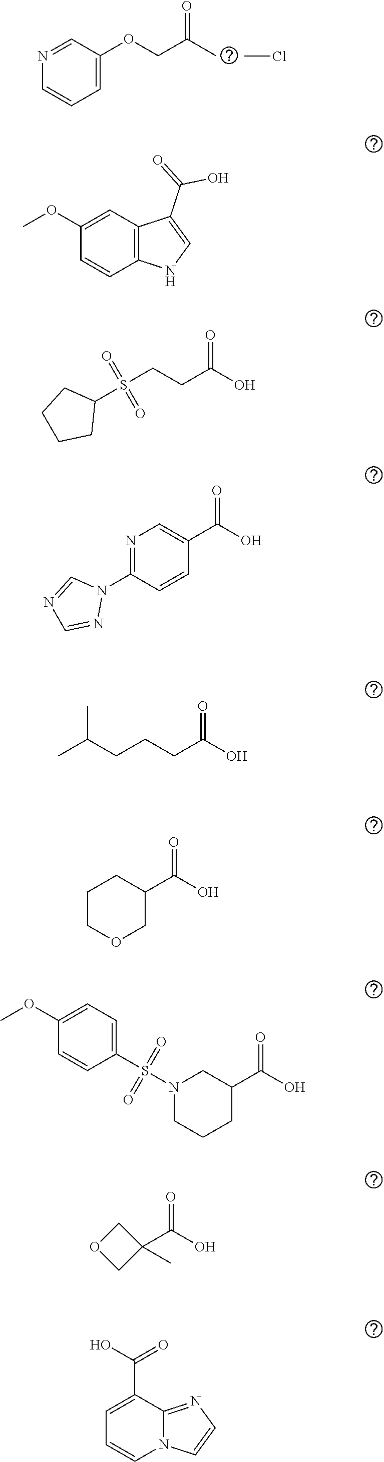

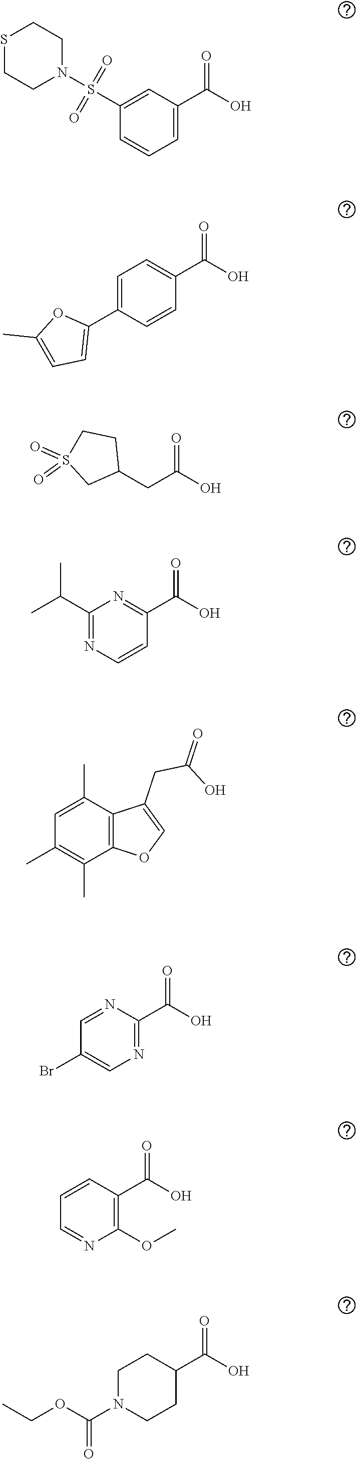

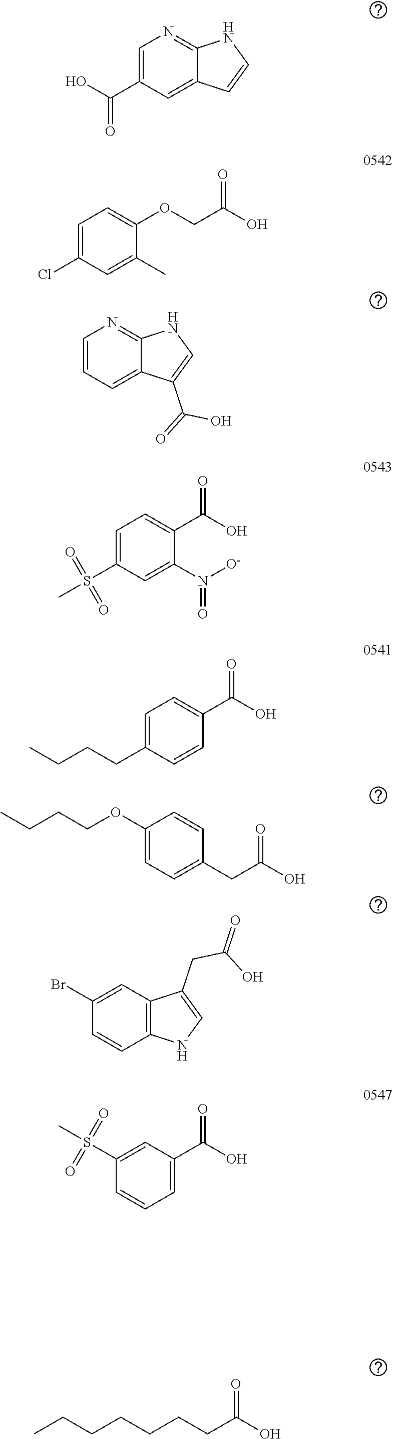

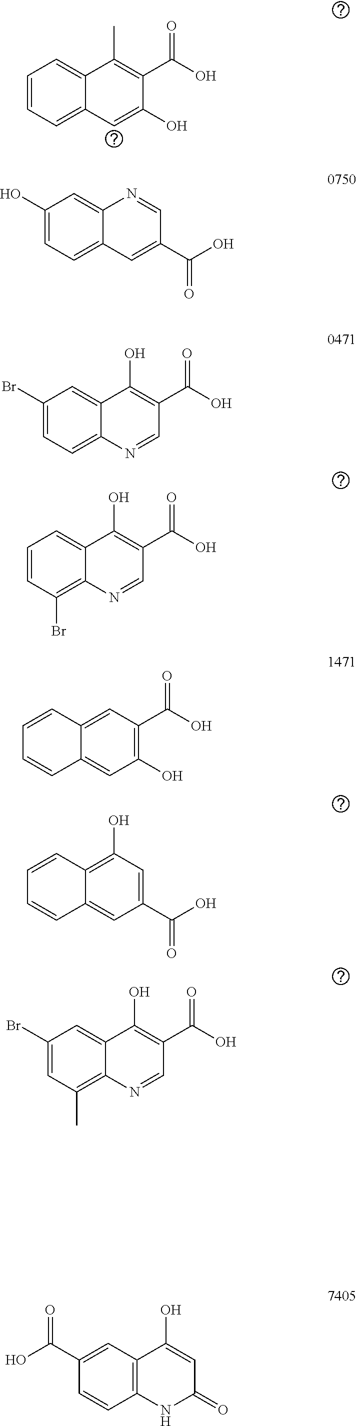

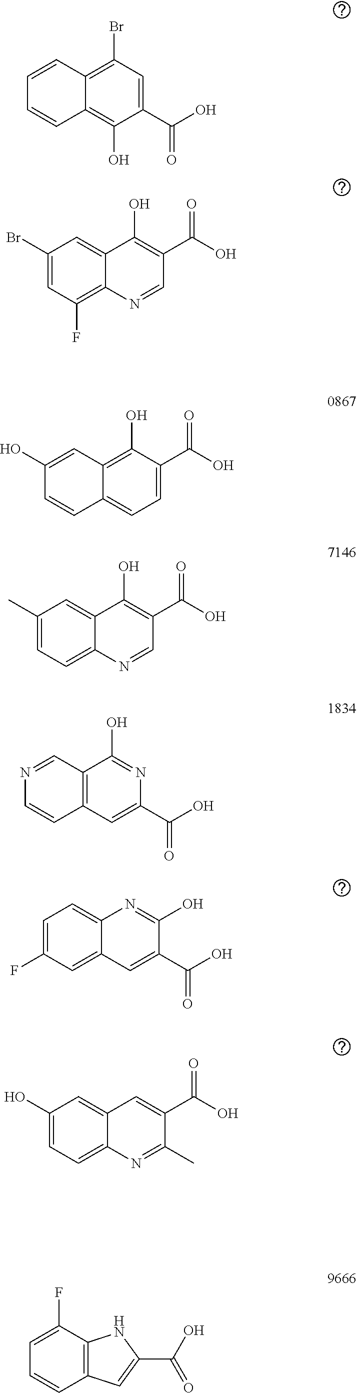

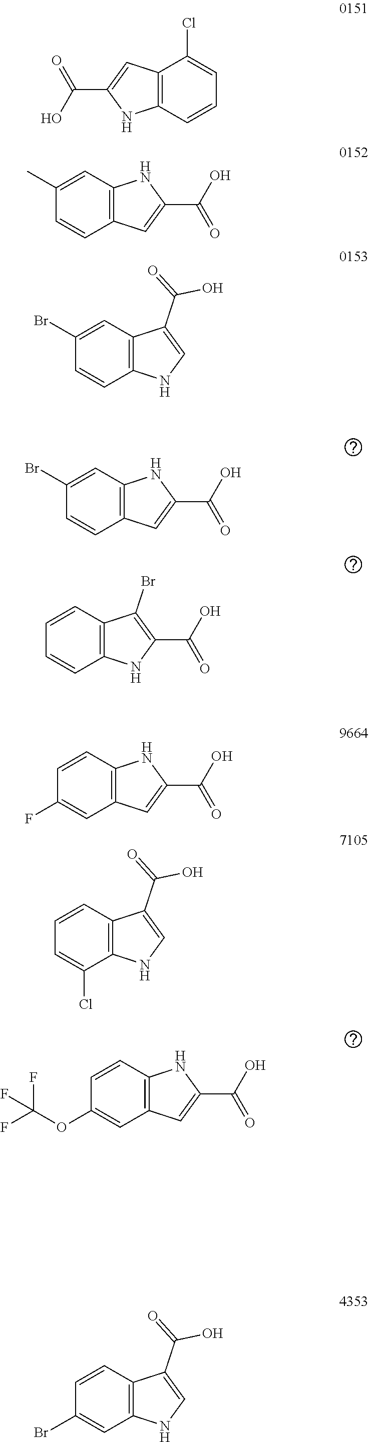

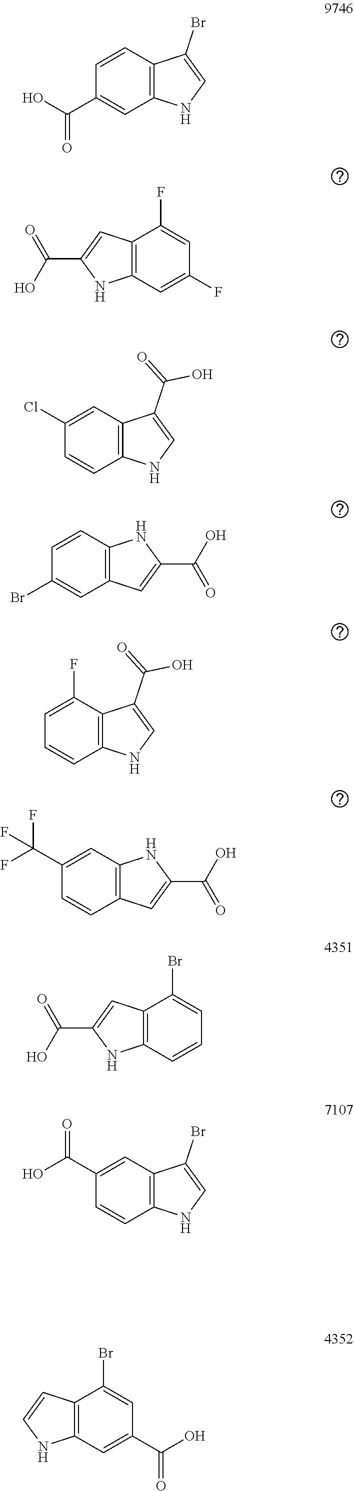

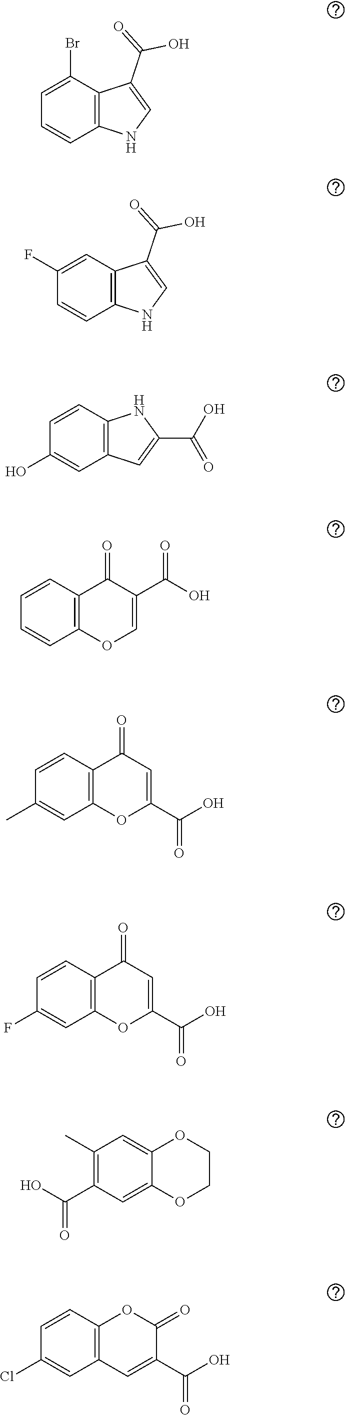

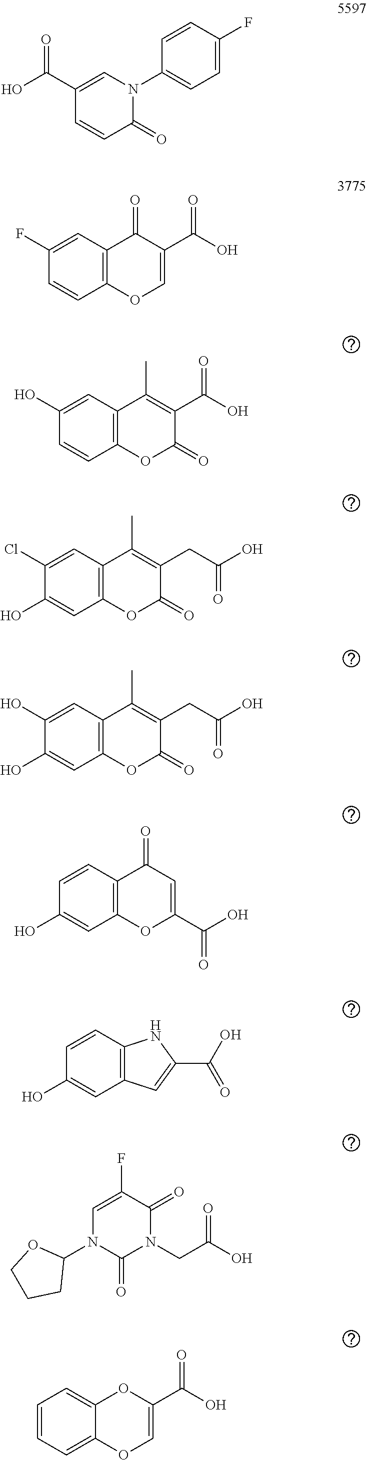

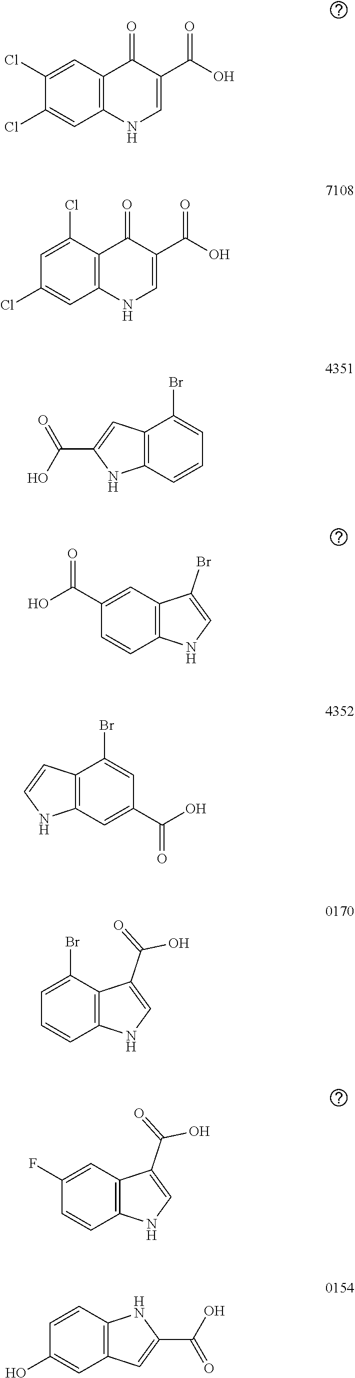

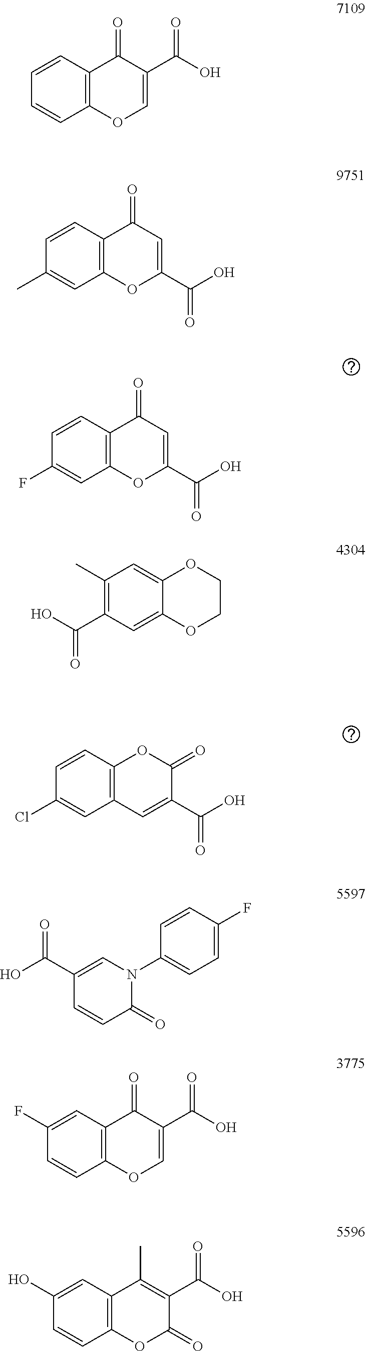

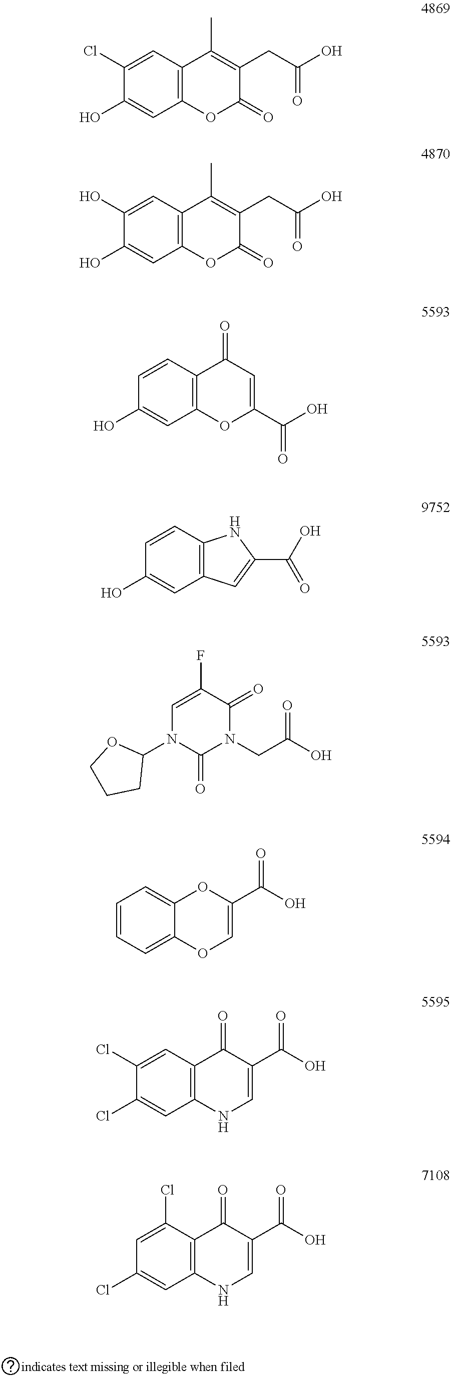

In yet more particular embodiments, the compound of formula (I) is:

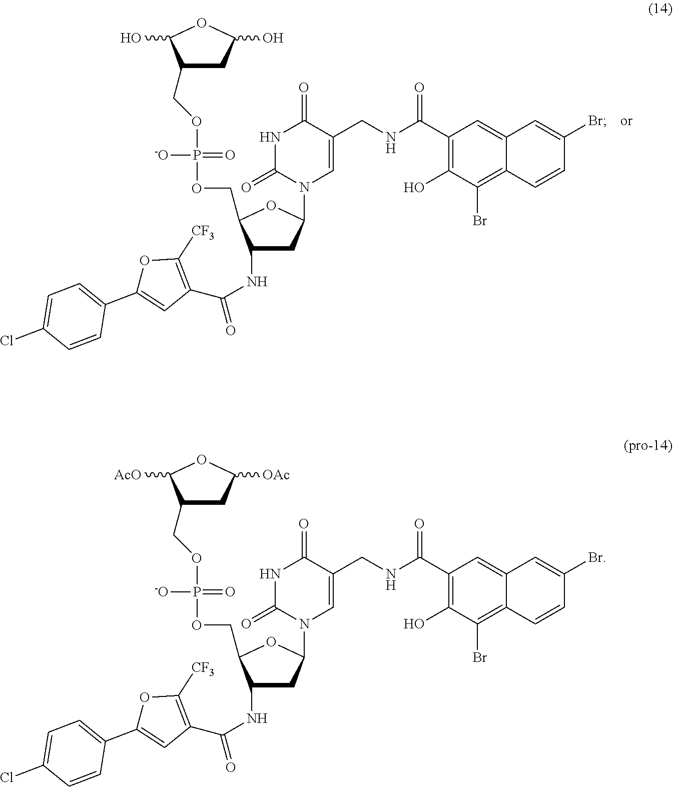

In other embodiments, the presently disclosed subject matter provides a method for irreversibly inhibiting a DNA polymerase, the method comprising administering to the subject a therapeutically effective amount of a compound of Formula (I), or a pharmaceutically acceptable salt thereof. In certain embodiments, the DNA polymerase comprises a DNA repair enzyme.

As used herein, the term “DNA repair enzyme” includes an enzyme that can repair changes or mutations in DNA and restore the DNA to its original state. The presently disclosed method is applicable for various DNA repair enzymes.

As used herein, the terms “lyase activity” or “polymerase activity” means an activity that involves the removal elimination of a group or the addition of a nucleotide or nucleotide analogue to a nucleic acid chain, respectively. Accordingly, in some embodiments, the compound inhibits the lyase and the polymerase activities of the DNA repair enzyme.

The presently disclosed methods can be used to inhibit DNA repair enzymes with lyase and/or polymerase activities. Non-limiting examples of DNA repair enzymes include DNA polymerase β (UniProt Accession No. P06746, for example), 5′-deoxyribose-5-phosphate lyase Ku70 (UniProt Accession No. P12956, for example), Endonuclease III-like protein 1(UniProt Accession No. P78549, for example), DNA polymerase λ (Pol λ), DNA polymerase θ (Pol θ), and the like. In some embodiments, the DNA repair enzyme is selected from the group consisting of DNA polymerase β, 5′-deoxyribose-5-phosphate lyase Ku70, and Endonuclease III-like protein 1.

In some embodiments, the DNA repair enzyme is DNA polymerase β. In certain embodiments, the compound of Formula (I) inhibits the lyase and polymerase activities of the DNA polymerase.

As used herein, the term “inhibit” or “inhibits” has at least two meanings. It may mean to decrease, suppress, attenuate, diminish, arrest, or stabilize the development or progression of a disease, disorder, or condition, the activity of a biological pathway, or a biological activity such as cancer, e.g., by at least 10%, 20%, 30%, 40%, 50%, 60%, 70%, 80%, 90%, 95%, 98%, 99%, or even 100% compared to an untreated control subject, cell, biological pathway, or biological activity. It will be appreciated that, although not precluded, treating a disease, disorder or condition does not require that the disease, disorder, condition or symptoms associated therewith be completely eliminated. The term “inhibit” or “inhibits” may also mean to decrease, suppress, attenuate, diminish, or arrest the activity of an enzyme, which is a biological molecule that accelerates both the rate and specificity of a metabolic reaction. An “inhibitor” is a molecule that inhibits the activity of an enzyme. An “irreversible inhibitor” usually covalently modifies an enzyme and therefore the inhibition cannot be reversed. Irreversible inhibitors may act at, near, or remote from the active site of an enzyme.

In particular embodiments, the subject has cancer. A “cancer” in a subject refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. Proliferation of a cancer cell can include an increase in the number of cells as a result of cell growth and cell division. A cancer can include, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas.

Without wishing to be bound to any one particular theory, it is believed that inhibiting Pol β activity can be cytotoxic to a cancer cell. In more particular embodiments, inhibiting the DNA polymerase treats, inhibits, delays, or prevents the spread of the cancer in the subject. In yet more particular embodiments, the method further comprises treating, inhibiting, delaying, or preventing the spread of the cancer by inhibiting at least one cancer cell involved in one or more biological processes selected from the group consisting of cell migration, cell growth, cell adhesion, angiogenesis, cancer cell invasion, apoptosis, tumor formation, tumor progression, metastasis, degradation of the extracellular matrix, pericellular proteolysis, activation of plasminogen, and changes in the levels of an extracellular protease.

In certain embodiments, the method further comprises administering to the subject a DNA damaging agent. In particular embodiments, the DNA damaging agent is methyl methanesulfonate (MMS) of bleomycin. In certain embodiments, the DNA damaging agent is administered before or simultaneously with administration of the compound of Formula (I). In particular embodiments, the compound of formula (I) and the DNA damaging agent have a synergistic effect in treating a cancer.

The presently disclosed methods may further comprise administering to the subject a DNA damaging agent in combination with a compound of Formula (I). A “DNA damaging agent” is an agent that damages the DNA structure in some way, such as causing damage in the DNA bases or its sugar phosphate backbone or causing the formation of covalent bonds between the DNA and at least one protein or between two DNA strands. The DNA damage may affect DNA-histone and DNA-transcription factor interactions, interactions with other proteins and may impact DNA packing, cell division, replication and/or transcription of the DNA.

As provided in more detail herein below, the presently disclosed compounds potentiate the cytotoxicity of a DNA damaging agent whose effects would require repair by Pol β. In particular embodiments, the DNA damaging agent is administered before, simultaneously, or after administration of the compound of Formula (I), or combinations thereof.

A DNA damaging agent may include, for example, an agent that alkylates DNA or oxidatively damages DNA. In particular embodiments, the DNA damaging agent is bleomycin that oxidizes the 2′-deoxyribose backbone or a methylating agent. In yet more particular embodiments, the methylating agent is methyl methanesulfonate (MMS). Other methylating agents include, but are not limited to, N-methyl-N-nitrosourea (MNU), N-methyl-N′-nitro-N-nitrosoguanidine (MNNG), methylalkylnitrosamines, including dimethylnitrosamine (DMN), methylazoxymethanol (MAM), 4-(methyl-nitrosamino)-1-(3-pyridyl)-1-butanone (NNK), temozolomide (4-methyl-5-oxo-2,3,4,6,8-pentazabicyclo[4.3.0]nona-2,7,9-triene-9-carboxamide (TMZ)), dacarbazine (5-(3,3-Dimethyl-1-triazenyl)imidazole-4-carboxamide), procarbazine (N-Isopropyl-4-[(2-methylhydrazino)methyl]benzamide), and 4-nitroquinoline-1-oxide.

By “in combination with” is meant the administration of a compound of Formula (I), or other compounds disclosed herein, with one or more therapeutic agents, e.g., a DNA damaging agent, either simultaneously, sequentially, or a combination thereof. Therefore, a cell or a subject administered a combination of a compound of Formula (I), or other compounds disclosed herein, can receive a compound of Formula (I), or other compounds disclosed herein, and one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of Formula (I), or other compounds disclosed herein, and one or more therapeutic agents are administered simultaneously, they can be administered to the cell or administered to the subject as separate pharmaceutical compositions, each comprising either a compound of Formula (I), or other compounds disclosed herein, or one or more therapeutic agents, or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered compound is not diminished by the sequential, simultaneous or separate administration of the subsequent compound(s).

In particular embodiments, a compound of Formula (I) and the DNA damaging agent work synergistically to inhibit a cancer cell. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of Formula (I) and another agent, e.g., an alkylating agent, such as methyl methanesulfonate (MMS), is greater than the sum of the biological activities of the compound of Formula (I) and the other agent when administered individually.

Synergy, expressed in terms of a “Synergy Index (SI),” generally can be determined by the method described by F. C. Kull, et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, e.g., a combination of components A and B, which produced an end point in relation to the combination;
Q_{B} is the concentration of a component B, acting alone, which produced an end point in relation to component B; and
Q_{b} is the concentration of component B, in the mixture, which produced an end point in relation to the combination.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture.

Thus, a “synergistic combination” has an activity higher that what can be expected based on the observed activities of the individual components when used alone. Further, a “synergistically effective amount” of a component refers to the amount of the component necessary to elicit a synergistic effect.

In other embodiments, the presently disclosed subject matter provides a method for inhibiting a cancer cell, the method comprising contacting the cancer or noncancerous cell with a compound of Formula (I), or a pharmaceutically acceptable salt thereof, in an amount effective to irreversibly inhibit a DNA polymerase.

By “contacting”, it is meant any action that results in a therapeutically effective amount of at least one presently disclosed compound physically contacting at least one cell comprising a DNA polymerase. The method can be practiced in vitro or ex vivo by introducing, and preferably mixing, the compound and cell comprising a DNA polymerase in a controlled environment, such as a culture dish or tube. The method can be practiced in vivo, in which case contacting means exposing at least one cell comprising a DNA polymerase in a subject to a therapeutically effective amount of at least one compound of the presently disclosed subject matter, such as administering the compound to a subject via any suitable route. According to the presently disclosed subject matter, contacting may comprise introducing, exposing, and the like, the compound at a site distant to the cell comprising a DNA polymerase to be contacted, and allowing the bodily functions of the subject, or natural (e.g., diffusion) or man-induced (e.g., swirling) movements of fluids to result in contact of the compound and cell comprising a DNA polymerase(s). In some embodiments, the method may inhibit a DNA polymerase in vitro, in vivo, or ex vivo.

In other embodiments, the presently disclosed subject matter provides a pharmaceutical composition including one compound of formula (I), alone or in combination with one or more additional therapeutic agents in admixture with a pharmaceutically acceptable excipient. One of skill in the art will recognize that the pharmaceutical compositions include the pharmaceutically acceptable salts of the compounds described above.

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, base addition salts including, but not limited to, sodium, potassium, calcium, ammonium, triethylammonium, organic amino, magnesium, or similar salts. Other pharmaceutically acceptable salts may be found in, for example, Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Depending upon the particular condition, or disease state, to be treated or prevented, additional therapeutic agents, which are normally administered to treat or prevent that condition, may be administered together with the inhibitors of this disclosure. For example, chemotherapeutic agents or other antiproliferative agents may be combined with the inhibitors of this disclosure to treat proliferative diseases and cancer. Examples of known chemotherapeutic agents include, but are not limited to, adriamycin, bleomycin, dexamethasone, vincristine, cyclophosphamide, fluorouracil, topotecan, taxol, interferons, and platinum derivatives.

The additional agents may be administered separately, as part of a multiple dosage regimen, from the inhibitor-containing composition. Alternatively, these agents may be part of a single dosage form, mixed together with the inhibitor in a single composition.

### II. Synthetic Lethality Between Homologous Recombination and DNA Polymerase Beta Repair Pathways

In some embodiments, the presently disclosed subject matter establishes a synthetic lethal interaction between BRCA1 and DNA polymerase β. Cells that are deficient in BRCA1 are prone to become cancerous. The majority of subjects with mutations in this gene develop cancer in their lifetime. Identifying synthetic lethal interactions with BRCA1 is an important approach to treating such cancers. This approach is exemplified by the targeting of PARP1 and the development of Olaparib and other chemotherapeutics. There is a need, however, for additional synthetic lethal partners for BRCA1 because not all cells respond to PARP inhibitors and cells develop resistance. Selective inhibitors of DNA polymerase D are disclosed in International PCT Patent Application Publication No. WO2018034987 for DNA Polymerase Beta Inhibitors, to Greenberg et al., published Feb. 22, 2018, which is incorporated herein by reference in its entirety.

In some embodiments, a representative polymerase θ inhibitor and siRNA are used to demonstrate that there is a synthetic lethal interaction between BRCA1 and DNA polymerase beta. This observation makes DNA polymerase β an attractive anticancer target.

Accordingly, in yet other embodiments, the presently disclosed subject matter provides a method for inducing a synthetic lethality in a breast cancer type 1 (BRCA1)-deficient cancer cell, the method comprising inhibiting DNA polymerase β by administering a compound of Formula (I) to the cell.

### III. Methods for Identifying One or More DNA Polymerase Inhibitors

In other embodiments, the presently disclosed subject matter provides a method for identifying one or more DNA polymerase inhibitors, the method comprising: (a) contacting one or more candidate polymerase inhibitors with a DNA polymerase to form an inhibitor/polymerase solution; (b) contacting the inhibitor/polymerase solution with a ternary DNA template and deoxynucleotide triphosphate(s) (dNTP), (c) measuring polymerization can be measured using fluorescently labeled DNA primer. Polymerization can also be detected using reagents, such as Sybr Gold that selectively bind to duplex DNA and fluoresce upon binding.

In some embodiments, a ternary DNA template is fluorescently labeled at a 3′-terminus. In certain embodiments, the fluorescent label comprises TAMRA (carboxytetramethylrhodamine). In some embodiments, a ternary DNA template is labeled at a 5′-terminus with a black hole quencher.

In particular embodiments, the DNA polymerase comprises DNA polymerase β (pol β).

In certain embodiments, an ability to prevent an increase in fluorescence indicates that the one or more candidate DNA polymerase inhibitors is a DNA polymerase inhibitor. In certain embodiments, the method further comprises measuring a rate constant of fluorescence activity to determine a relative rate of DNA polymerase activity of the one or more candidate DNA polymerase inhibitors. In certain embodiments, the method further comprises incubating the inhibitor/polymerase solution for a period of time before contacting it with the ternary DNA template and deoxythymidine triphosphate (dTTP). In certain embodiments, the method further comprises diluting the inhibitor/polymerase solution before contacting it with the ternary DNA template and deoxythymidine triphosphate (dTTP).

### IV. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula I are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Description of compounds of the present disclosure is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, methoxy, diethylamino, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons). In particular embodiments, the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, iso-pentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₆ straight-chain alkyls, including C₁, C₂, C₃, C₄, C₅, and C₆ alkyl. In other embodiments, “alkyl” refers, in particular, to C₁₋₆ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)R′, —C(O)NR′, —NR′R″, —OR′, —SR, and/or —SO₂R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, pentenyl, hexenyl, octenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents,” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently.

The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

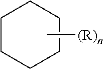

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

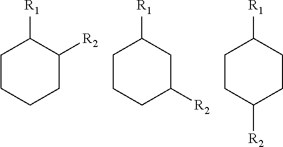

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxy, n-hexoxy, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The term “pharmaceutically acceptable salts” is meant to include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like {see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Proinhibitors of the compounds described herein are prodrugs that after undergoing chemical changes under physiological conditions, act as inhibitors (molecules that inhibit the activity of an enzyme).

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

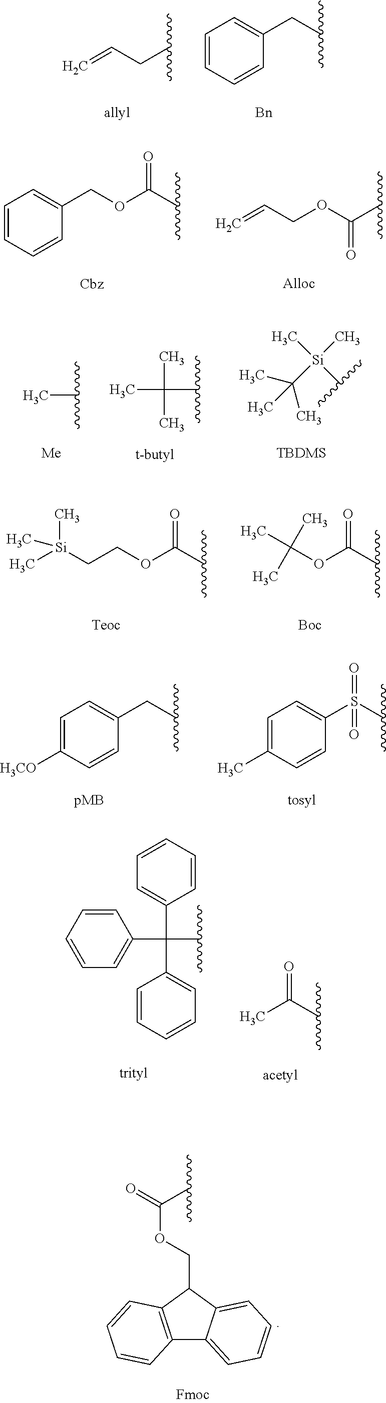

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition (e.g., a disease, condition, or disorder related to cancer), or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Selective Inhibition of DNA Polymerase β by a Covalent Inhibitor

### 1.1 Overview

DNA polymerase β (Pol β) plays a vital role in DNA repair and has been closely linked to cancer. Selective inhibitors of this enzyme are lacking, however. Inspired by DNA lesions produced by antitumor agents that inactivate Pol β, the development of covalent small-molecule inhibitors of this enzyme was undertaken. Using a two-stage process involving chemically synthesized libraries, a potent irreversible inhibitor (14) of Pol β (K₁=1.8±0.45 μM, k_{inact}=7.0±1.0×10⁻³ s⁻¹) was identified. Inhibitor 14 selectively inactivates Pol β over other DNA polymerases. LC-MS/MS analysis of trypsin digests of Pol β treated with 14 identified two lysines within the polymerase binding site that are covalently modified, one of which was previously determined to play a role in DNA binding. Fluorescence anisotropy experiments show that pretreatment of Pol β with 14 prevents DNA binding. Experiments using a pro-inhibitor (pro-14) in wild type mouse embryonic fibroblasts (MEFs) indicate that the inhibitor (5 μM) is itself not cytotoxic but works synergistically with the DNA alkylating agent, methyl methanesulfonate (MMS) to kill cells. Moreover, experiments in Pol β null MEFs indicate that pro-14 is selective for the target enzyme. Finally, pro-14 also works synergistically with MMS and bleomycin to kill HeLa cells. The results suggest that pro-14 is a potentially useful tool in studies of the role of Pol β in disease.

DNA polymerase j (Pol V) plays a vital role in base excision repair (BER, Scheme 1) in the nucleus and mitochondria. Friedberg et al., 2006; Wu et al., 2014; Beard et al., 2014; Prasad et al., 2017; Sykora et al., 2017. Pol β also contributes to double strand break repair via the alternative nonhomologous end joining pathway. Ray et al., 2018. The enzyme is up-regulated and/or mutated in many human cancers, such as colon cancer in which the mutation rate reaches approximately 40%. Donigan et al., 2012. Pol β has been postulated to be synthetic lethal in HR-deficient cancer cells, but this has not been verified experimentally. Nickoloff et al., 2017. Although Pol β inhibitors have been reported, there is a need for molecules that are selective and efficacious in cells. Wojtaszek et al., 2019; Gowda et al., 2017; Strittmatter et al., 2011; Strittmatter et al., 2013; Jaiswal et al., 2015; Arian et al., 2014; Paul et al., 2017; Paul et al., 2018; Goellner et al., 2012. The presently disclosed subject matter, in part, provides a selective, covalent Pol β inhibitor that acts on the enzyme in human cells.

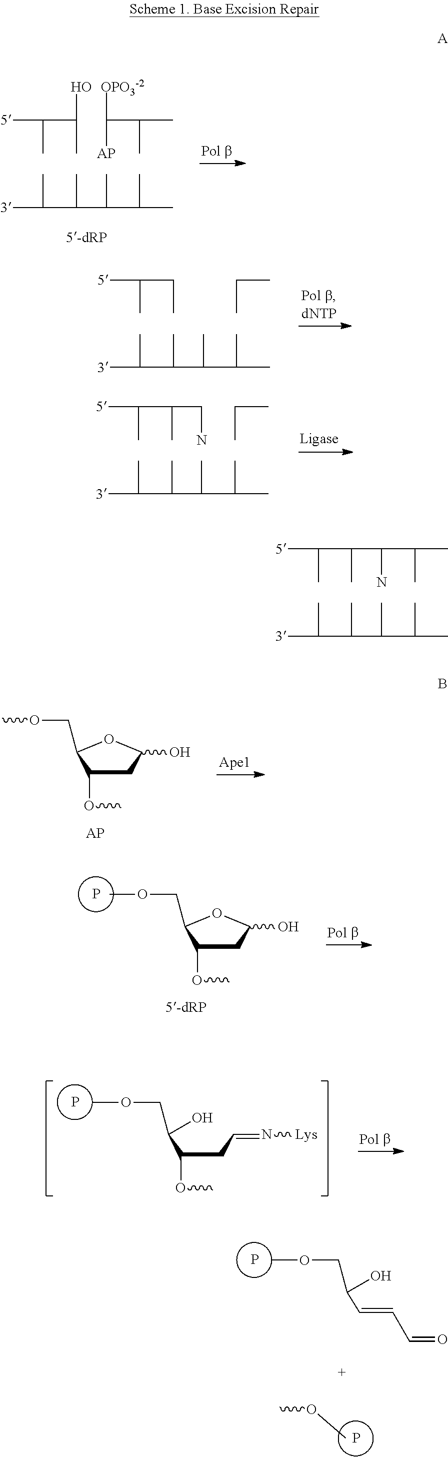

Pol β also is one of a handful of bifunctional DNA polymerases (Scheme 1A) possessing polymerase and lyase activity (Scheme 1B). The activities are associated with separate binding sites and 39 kDa Pol β can be divided into as 31 kDa polymerase domain and a shorter 8 kDa amino terminus domain that harbors the lyase activity. In short patch BER, the lyase activity produces a single nucleotide gap via Schiff base formation with the 5′-deoxyribose phosphate (5′-dRP) generated by incision of the 5′-phosphate of an abasic site (AP) by apurinic endonuclease 1 (Ape1). When Pol β binds oxidized abasic sites, such as DOB, attempted excision via initial nucleophilic attack results in covalently modified, inactivated enzyme (Scheme 2). Guan and Greenberg, 2010; guan et al., 2010; Jacobs et al., 2011.

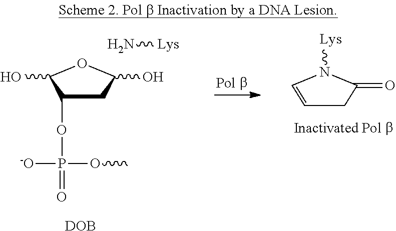

Inactivation of Pol β by oxidized abasic sites served as an inspiration for the design of mechanism-based irreversible inhibitors (1, 2):

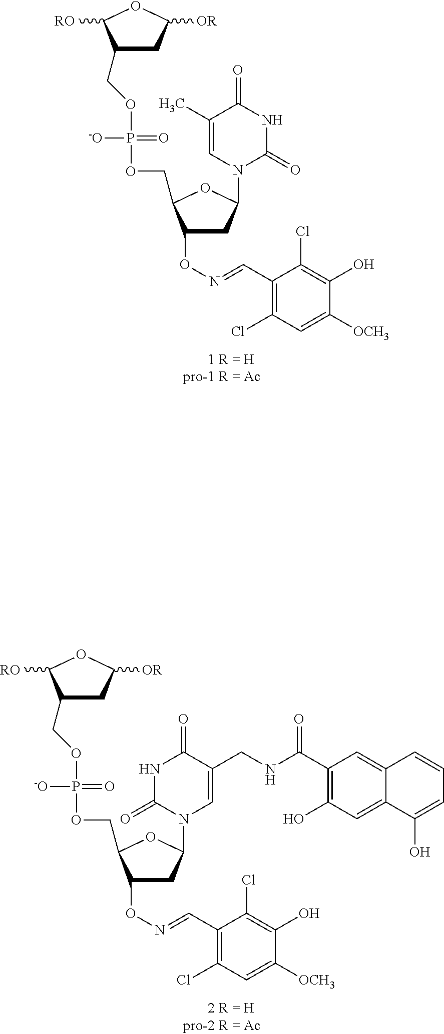

These molecules contain a 1,4-dioxobutane group linked to a 5′-phosphorylated thymidine (1) or C5-derivative (2) via a methylene linker that reduces D-elimination from the dicarbonyl component. The substituents at the 3′- and C5-positions of the thymidine were obtained by screening libraries of molecules (<350). The pro-inhibitors (pro-1, pro-2) of both compounds work synergistically with DNA damaging agents to kill mammalian cells. Although 2 is approximately 50-times more active against Pol β than is 1, it is even more effective at inactivating Pol λ. Pol λ has biological function that is independent of Pol β, but the enzymes have overlapping activity and the former is frequently thought of as a back-up to Pol β in BER. Garcia-Diaz et al., 2001; Braithwaite et al., 2010; Braithwaite et al., 2005; Stevens et al., 2013. It is desirable to identify inhibitors that are selective for one enzyme over another.

### 1.2 Results and Discussion

### 1.2.1 Inhibitor Identification Strategy

A high nanomolar irreversible inhibitor of Pol β was previously identified via a two-step process. Arian et al., 2014; Paul et al., 2017; Paul et al., 2018. A library consisting of oximes at the 3-terminus of a nucleotide was screened in step one. The inhibitor (1) identified from this procedure was then used to synthesize a library of molecules in which structural diversity was introduced at the C5-methyl position of a thymine in the form of amides. To streamline the synthesis process and maximize the use of a stable carboxylic acid library, the process was started from readily available AZT (Scheme 3). In contrast to previous investigations that yielded 1 and 2, the 3-recognition element in this study is appended to the nucleotide core via an amide bond.

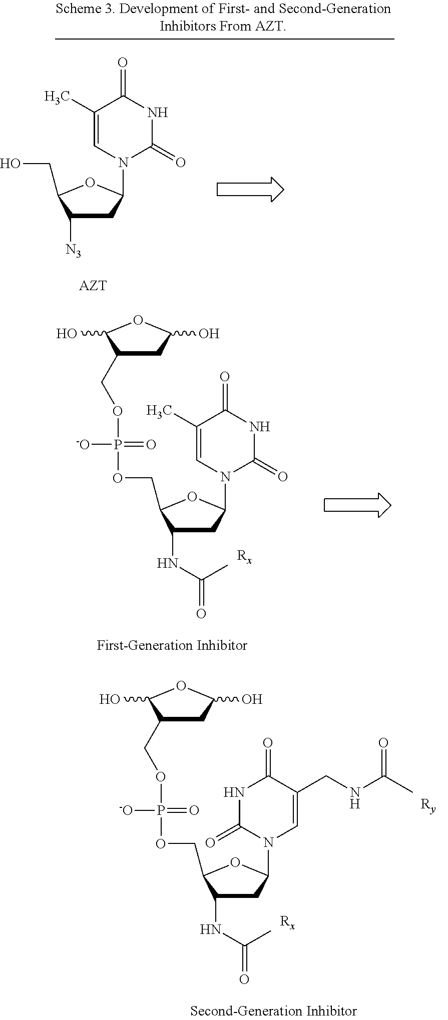

### 1.2.2 Identification of a First-Generation Irreversible Inhibitor

The precursor (6) for preparing the library was rapidly synthesized from AZT (Scheme 4).

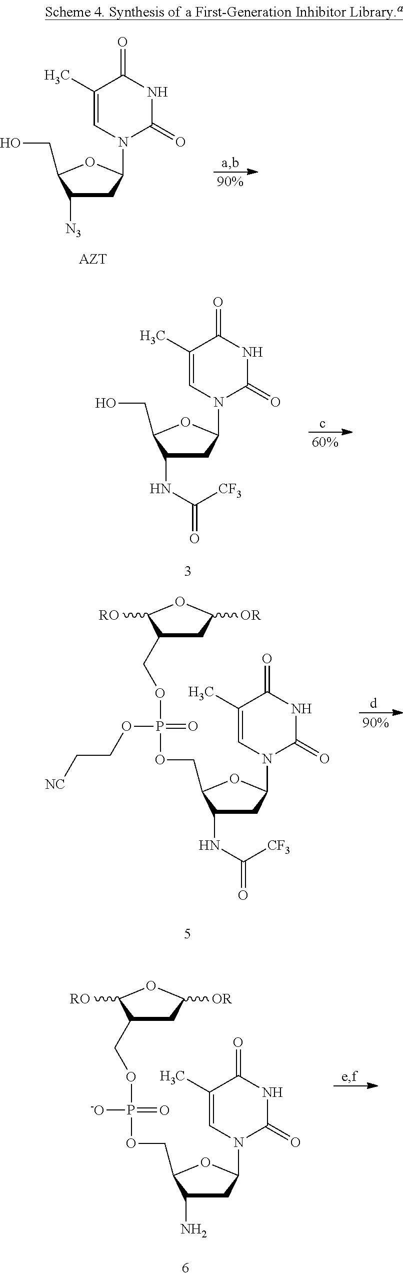

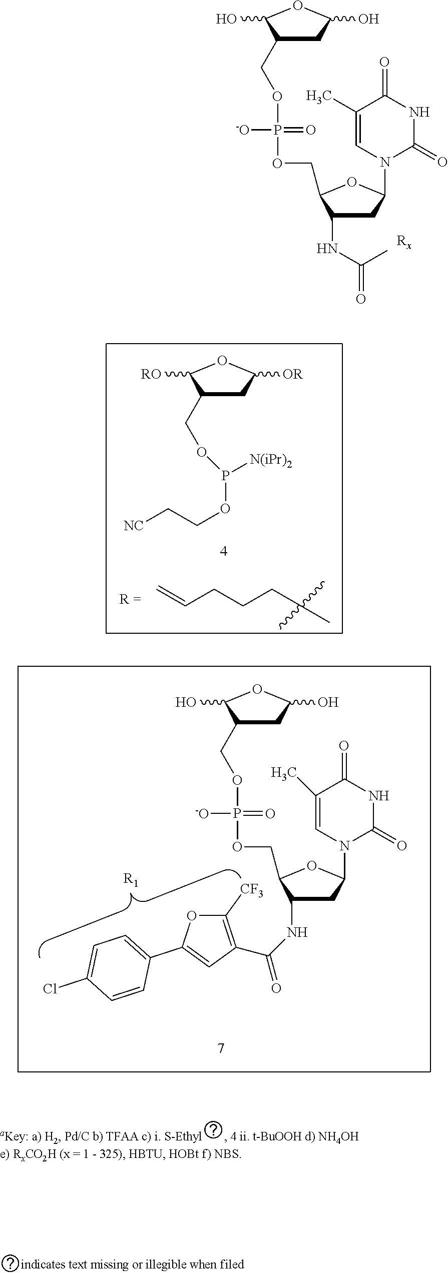

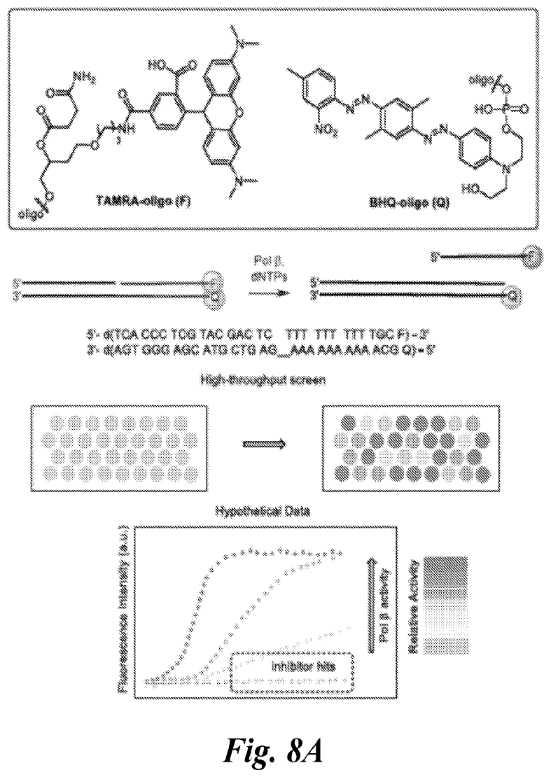

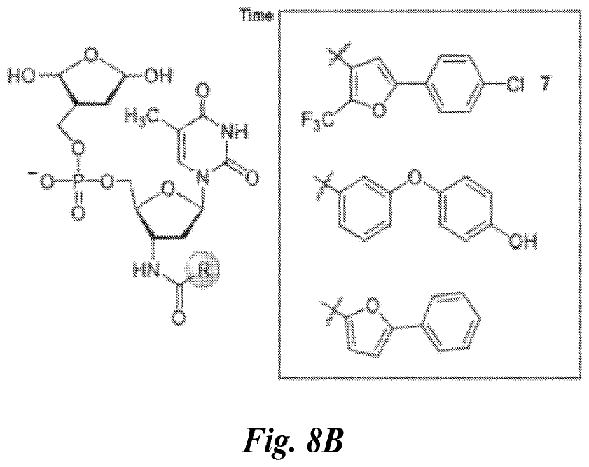

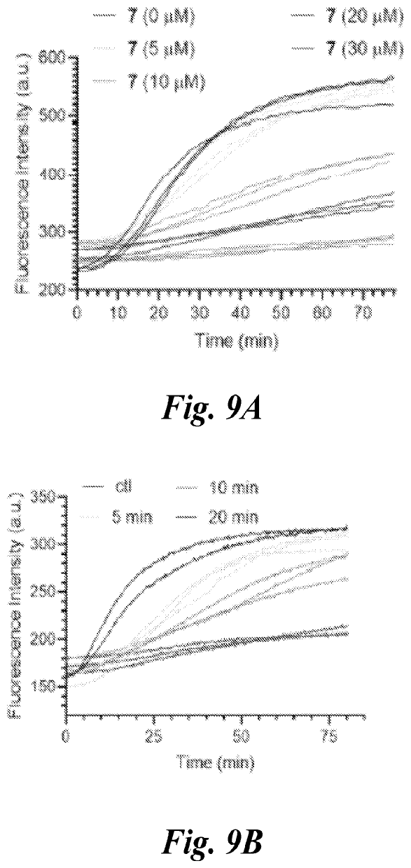

The azide was reduced and the resulting amine was protected as the trifluoroacetamide (3) prior to coupling with phosphoramidite 4, which bears the protected 1,4-dioxobutane warhead. The bis-pentene acetal was used to mask the warhead, because it could be cleaved rapidly under mild conditions that are compatible with other functional groups in the molecules that make up the library. Arian et al., 2014. Although it would have been more direct to reduce the azide after forming the phosphate bond, the slightly longer method avoided competing azide reduction by the phosphoramidite during coupling. Phosphoramidite 4 was prepared via phosphitylation of the corresponding alcohol as provided herein below. A mixture of diastereomers of 5 was deprotected using concentrated aqueous ammonium hydroxide at room temperature. Crude 6 was used to produce the library of first-generation inhibitor candidates. Utilizing a mixture of diastereomers of the DOB component was not a concern because it was expected to epimerize in water, following bis-pentene cleavage. The bis-acetal protected first-generation library was prepared in 384-well plates by coupling 6 with 1.4 equivalents of HBTU, HOBt and the corresponding carboxylic acids. The crude amides were then rapidly deprotected (5 min) in a different 384-well plate using 2.5 equivalents of N-bromosuccinimide at 4° C. and quenched with sodium thiosulfate. The inhibitor candidates were immediately screened for Pol β inhibition using a strand displacement assay in which the displaced oligonucleotide was fluorescently labeled at its 3′-terminus with TAMRA (carboxytetramethylrhodamine) and the template was labeled at its 5′-terminus with black hole quencher (FIG. 8A). Arian et al., 2014; Dorjsuren et al., 2009. Screening reactions containing Pol β (100 nM) and inhibitor (25 μM) were preincubated (30 min) prior to diluting 10-fold and reacting with the ternary DNA substrate and deoxythymidine triphosphate (dTTP). Initial hits were identified based upon their ability to prevent an increase in fluorescence. These molecules (3) were resynthesized from 6 and screened side-by-side with a control experiment containing all reagents except 6 to eliminate false positives (FIG. 8B). Product 7 was identified as the most promising inhibitor and its bis-pentene acetal was resynthesized and purified to confirm its activity (FIG. 9A). Complete loss of Pol β strand displacement activity was observed following a 20 min preincubation with 15 μM 7. The diminution of strand displacement activity was dependent upon preincubation time, indicating that 7 irreversibly inhibits Pol β (FIG. 9B), and was carried forward in the search for a second-generation inhibitor (Scheme 3).

### 1.2.3 Identification of a Second-Generation Irreversible Inhibitor

The primary amine designated for introducing structural diversity at the C5-pyrimidine position was incorporated by reacting the crude bromide obtained from 8 with concentrated aqueous ammonium hydroxide in ethanol cosolvent (Scheme 5).

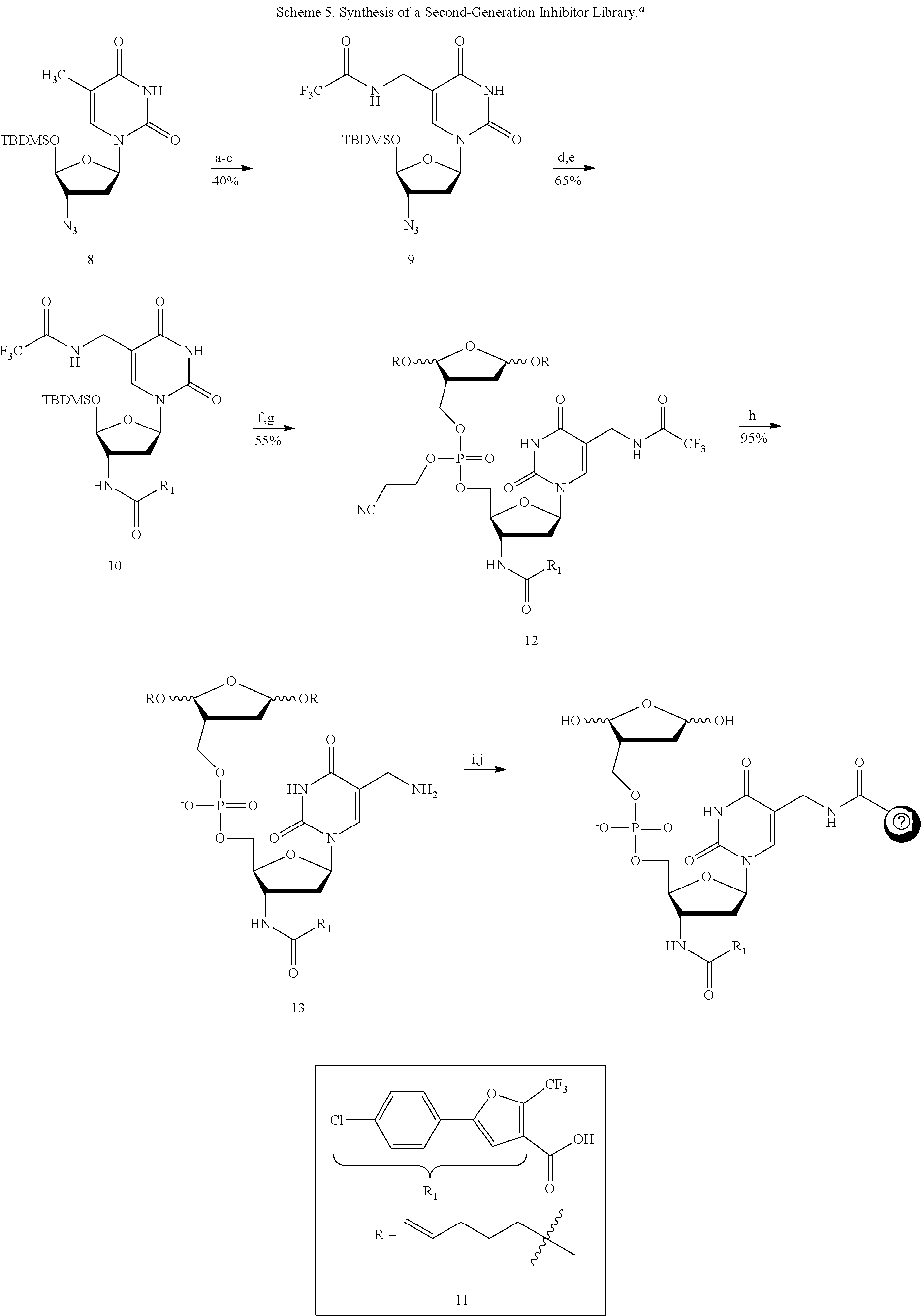

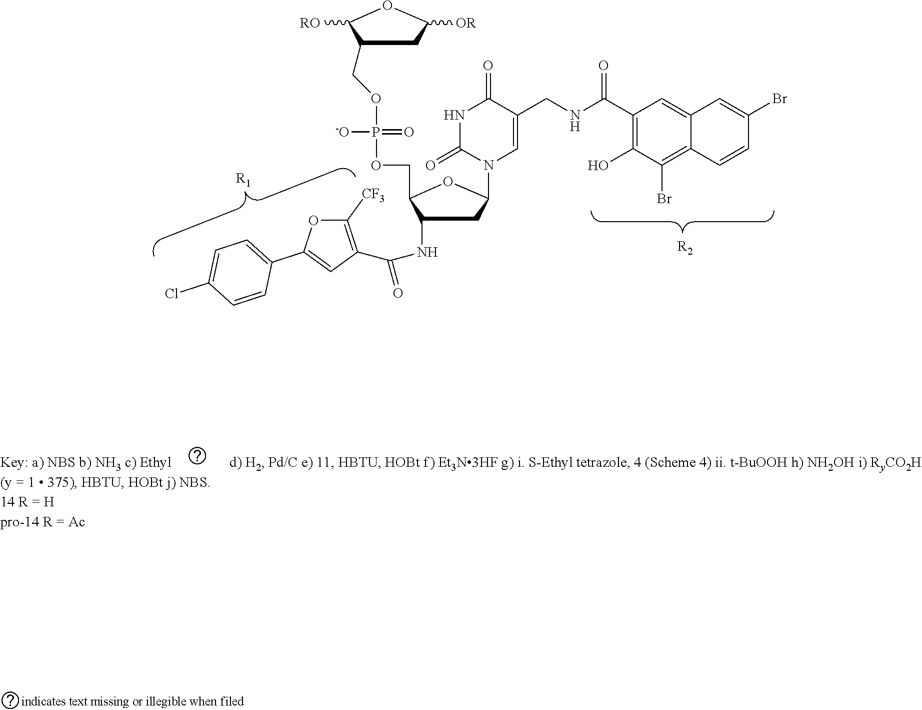

Following protection of the primary amine as the trifluoroacetamide, the azide in 9 was reduced and the amine coupled with the carboxylic acid (11) that provided first-generation inhibitor 7. Upon deprotection of the C5′-alcohol in 10, the nucleoside was coupled with phosphoramidite 4. The nucleoside was used in slight excess (1.2 equivalents) to minimize phosphoramidite by-products, which were difficult to separate from 12. Cleavage of the trifluoroacetamide and β-cyanoethyl protecting groups, as described for preparing 6 (Scheme 4), provided the substrate (13) for preparing the second-generation inhibitor library, and was purified by column chromatography (Scheme 5).

Precursor 13 was coupled with a library of carboxylic acids, followed by removal of the pentene acetal protecting groups, as described above for screening the first-generation library. Inhibitor screening was carried out at 700 nM (crude) inhibitor candidates, as opposed to 25 μM for the first-generation inhibitor. From this library, 14 was selected as the most promising candidate, after carrying out the control experiments described for the evaluation of the first-generation library. Inhibitor 14 was independently synthesized from 13. Coupling was carried out using the corresponding NHS-ester (45%) because reaction of the corresponding free acid provided the desired product in low yield. Following purification of the bis-pentenyl acetal precursor, 14 was generated on an as needed basis.

### 1.2.4 Irreversible Inhibition of Pol β by 14

The quantitative effect of 14 on Pol β activity at pH 7.4 was determined using the strand displacement assay employed to identify it. The time-dependent curves generated from the fluorescence-based assays were fit to an exponential growth region followed by a plateau. Zhang et al., 2011; Olson et al., 2017. Rate constants extracted from these data were used to determine relative rates of enzyme activity with and without inhibitor. The effect of 14 on polymerase activity was examined between 100 and 750 nM following preincubation with Pol β for between 2 and 20 min.

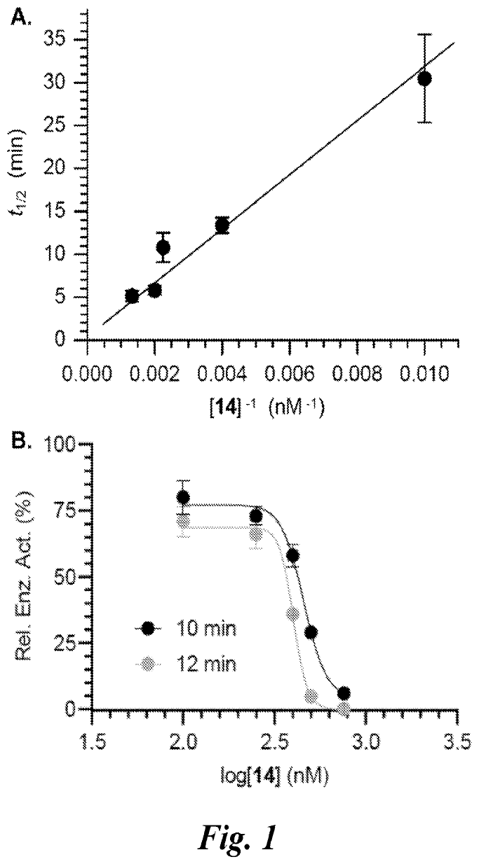

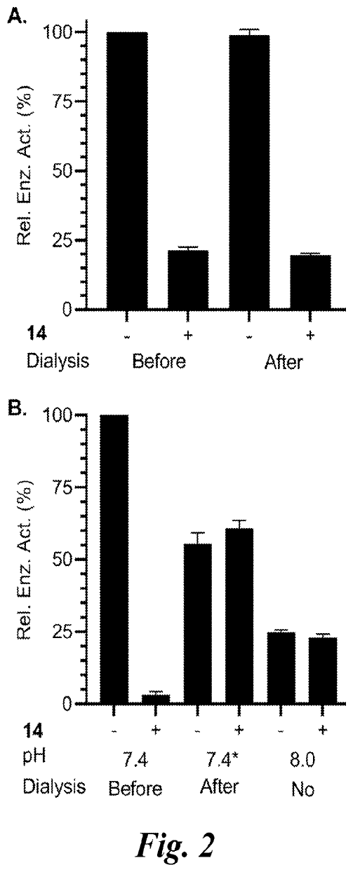

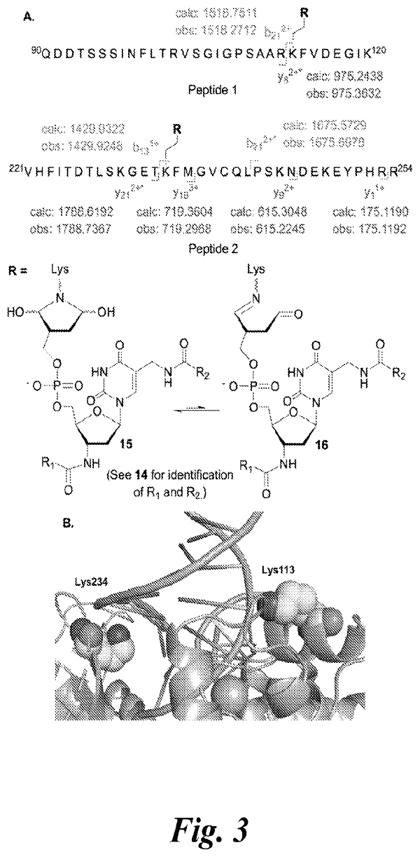

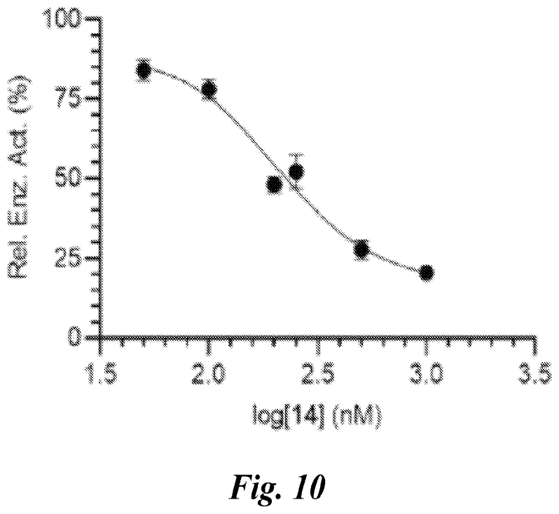

The K₁ (1.8±0.45 μM) and k_{inact} (7.0±1.0×10⁻³ s⁻¹) for 14 were extracted from a Ritz-Wilson plot (FIG. 1A). To the best of current knowledge, 14 is the only irreversible inhibitor of Pol β. Hence, k_{inact} cannot be compared to other molecules. The K₁, however, compares favorably to molecules such as honokiol, which is not selective for Pol β over Pol β. Gowda et al., 2017. Other molecules are more potent, but their selectivity is unsatisfactory or unknown. Goellner et al., 2012. The IC₅₀ (FIG. 1B) for Pol β inactivation by 14 was determined using 10 (458 nM) and 12 (409 nM) min preincubation times. In comparison, 2 exhibited an IC₅₀ value of 400 nM following 30 min preincubation. Paul et al., 2017; Paul et al., 2018. The IC₅₀ values of 2 and 14 cannot be compared directly because they were determined under different conditions (i.e., preincubation times and extent dilution following preincubation) To make a direct comparison, 14 was analyzed under the conditions employed for measuring the IC₅₀ of 2. Under the identical conditions, the IC₅₀ for 14 (204 nM) was approximately one half that of 2 (FIG. 10). Additional evidence for irreversible inhibition of Pol β by 14 was obtained by comparing the polymerase activity before and after dialysis (24 h, 4° C.), which revealed that the enzyme did not regain function following dialysis (FIG. 2A).

### 1.2.5 Inhibitor 14 Modifies Lysine Residues in the Polymerase Domain and Prevents DNA Binding by Pol β

The lack of an effect of 14 on Pol β lyase activity suggested that it interacts with one or more residues within the polymerase domain. LC-MS/MS analysis following trypsin digest of Pol β that was preincubated with 14 (300 nM, 30 min) supported this hypothesis. Fragment ion analysis of the two observed modified tryptic peptides revealed that Lys113 and Lys234 formed adducts with 14 (FIG. 3A). Peptide 1 contains a single internal lysine residue and an expected fragment containing a modification on K113 (y8*, Peptide 1, FIG. 3A) was detected. Peptide 2 (FIG. 3A) contains four internal lysine residues (K230, K234, K244, and K248) and required MS/MS fragmentation to identify the lysine modified by 14. Unmodified fragments containing K230 (b13), K244 (y18), and K248 (y9, y18) indicated these residues did not react with 14. A fragment containing a single modification was observed that contained K230 and K234 (b21*). In addition, another fragment that contained one modification and included K234, K244, and K248 (y21*). When considered in total, these data indicated K234 was the modified lysine in Peptide 2. The fragments observed in the gas phase correspond to the ring opened dehydrated forms (16), which would be expected to exist in equilibrium with the ring closed bis-hemiaminal isomers (15) in solution (FIG. 3A). (Imine formation is shown involving the C1-aldehyde but the data do not distinguish between this adduct or one involving the C4-aldehyde.) Based upon previous experiments that established the necessity of the 1,4-dicarbonyl moiety for inactivation, ring closed isomer 15 is believed to dominate in solution. Arian et al., 2014. Support for the proposed structure of the lysine adduct(s) is gleaned by examining the pH effect on inactivation by 14 (FIG. 2B). Inhibition is not observed when Pol β is treated with 14 at pH 8. In addition, enzyme activity is recovered following inactivation by 14 at pH 7.4 when dialyzed at pH 8 (FIG. 2B), but not pH 7.4 (FIG. 2A). It should be noted that the sample dialyzed at pH 8 (FIG. 2B, denoted pH 7.4*) showed complete inhibition prior to dialysis (FIG. 2B, denoted pH 7.4) and in both instances the preincubation and strand displacement kinetics were carried out at pH 7.4. Based upon these observations, the adduct(s) appears to be unstable at pH 8, consistent with the expected behavior of 15.

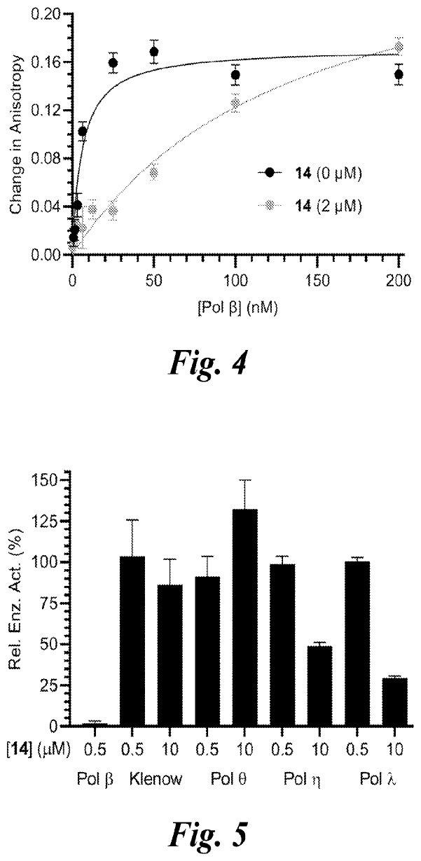

Identification of the modified lysines provided a mechanistic rationale for how 14 inactivates Pol β. Lys234 is important for DNA binding and is invariant across multiple species of Pol β. 44-46 In addition, analysis of the crystal structure of Pol β when complexed with DNA (PDB: 1BPX) indicates that Lys113 is within 10 Å of the DNA backbone (FIG. 3B). Sawaya et al., 1994; Yang et al., 2004; Sawaya et al., 1997. A fluorescently labeled ternary substrate containing a stable abasic site analogue (Table S1) was used to determine if Pol β incubation with 14 affected DNA binding (FIG. 4). Laverty et al., 2018. Fluorescence anisotropy measurements indicated DNA binding was significantly reduced following preincubation with 14 (2 μM). DNA binding interactions were most effectively disrupted at lower Pol β concentrations. Complete DNA binding was not observed until approximately 800 equivalents of protein were added. Together, fluorescence anisotropy and trypsin digestion experiments indicate that 14 irreversibly inhibits Pol D by covalently modifying lysine residues in the polymerase domain that result in compromised DNA binding.

### 1.2.6 Selective Inactivation of Pol β

Selective DNA polymerase inhibition is challenging. For instance, 2 is a more potent inhibitor of Pol λ than Pol β. Paul et al., 2017. The lack of selectivity between the two X-family polymerases has been observed in other reported Pol β inhibitors. The two enzymes share 32% sequence homology, which contributes to the difficulty in inhibiting one over the other. Garcia-Diaz et al., 2000. The potency of 14 for inhibiting Pol β was compared to its effect on a model replicative polymerase (Klenow exo-) and three mammalian polymerases involved in DNA repair, Pol θ, Pol η and Pol λ (FIG. 5). The effect of 14 on the respective enzymes were ascertained by determining the relative enzyme activity following preincubation with inhibitor versus in the absence of inhibitor.

Under conditions in which 14 (500 nM. 20 min preincubation) essentially completely inactivated Pol β, it had no effect (within experimental error) on Klenow exo-, Pol θ, Pol η or Pol λ. Increasing the concentration of 14 20-fold (10 μM) still had no effect on Klenow exo-or Pol θ activity. Pol η and Pol λ polymerase activities are reduced at 10 μM 14. Pol η, however, retains approximately 50% of its activity at this concentration and the activity of Pol λ is slightly more than 25% relative to untreated enzyme. Despite these effects, the existence of significant activity at 20 times the concentration of inhibit at which Pol β is completely inactivated indicates significant selectivity for this enzyme. It is not possible to rigorously compare the selectivity of 14 to many other Pol β inhibitors because it is a covalent inhibitor, while others such as honokiol are reversible inhibitors. Gowda et al., 2017. These data (FIG. 5), however, clearly indicate that 14 selectively inactivates Pol D over these four other polymerases.

### 1.2.7 Pro-14 Selectively Targets Pol β in Cells and Works Synergistically with DNA Damaging Agents

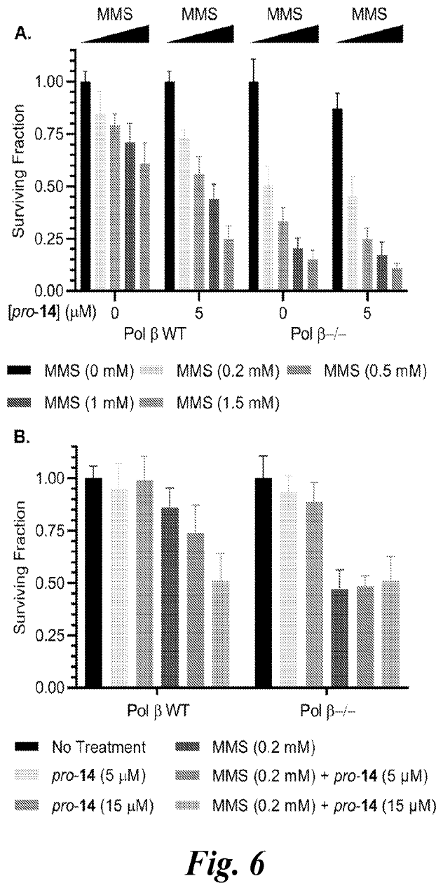

Pro-14 (5 μM) killed fewer than 5% wild type mouse embryonic fibroblasts (Pol β WT, MEFs) (FIG. 6a). The pro-inhibitor, however, enhanced the cytotoxicity of methyl methanesulfonate (MMS) when the concentration of alkylating agent was varied up to 1.5 mM. MMS activates BER by alkylating purines. Fu et al., 2012. The data indicate that pro-14 and MMS have a synergistic effect on MEF cytotoxicity. This is consistent with the ability of similarly designed pro-inhibitors to act synergistically with MMS. Arian et al., 2014; Paul et al., 2017; Paul et al., 2018.

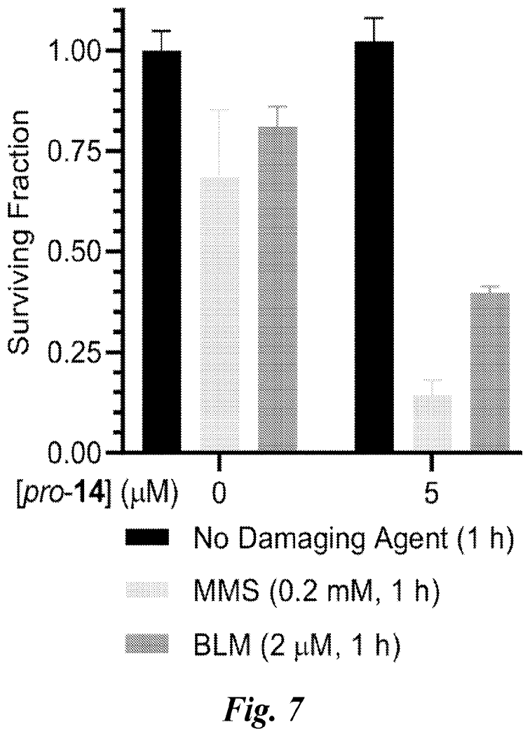

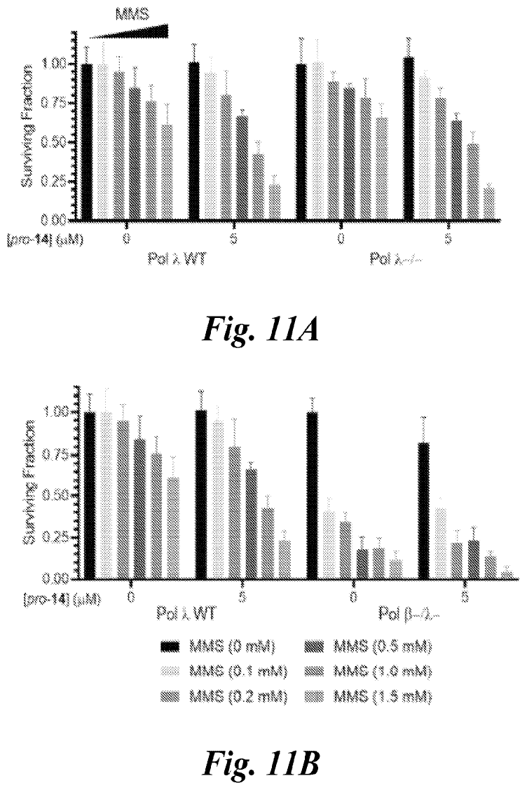

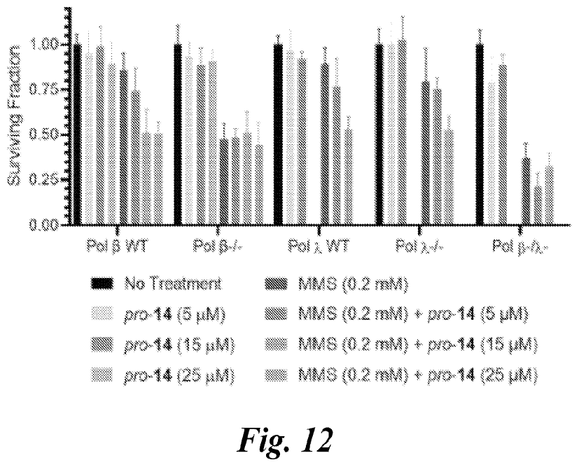

MMS was considerably more toxic to MEFs lacking Pol β (Pol β−/−). The presence of pro-14, however, did not result in additional cell death (FIG. 6A). Similarly, pro-14 did not increase the cytotoxicity of MMS in MEFs lacking pol β and/or pol λ (FIG. 11A and FIG. 11B). Importantly, these data indicate that pro-14 does not enhance MMS cytotoxicity by targeting enzymes other than Pol β. The selectivity of pro-14 on Pol β was examined at higher pro-14 concentrations in MEFs treated with 0.2 mM MMS. While the cytotoxicity was enhanced upon increasing pro-14 from 5 to 15 μM in WT MEFs, no additional cell death was observed in Pol β null (Pol β−/−) cells (FIG. 6B). Furthermore, even in the presence of 25 μM of pro-14, no additional cytotoxicity was detected in WT MEFs or Pol β null cells (FIG. 12). These data indicate that pro-14 does not target proteins that enhance MMS cytotoxicity when present at significantly higher concentrations than needed to synergistically kill cells containing Pol β. The synergistic effect of pro-14 on the cytotoxicity of MMS and the antitumor agent bleomycin (BLM) in HeLa cells also was examined (FIG. 7). Pro-14 (5 μM) exhibited <7% cytotoxicity to the HeLa cells. At this concentration, however, pro-14 enhanced the cytotoxicity of MMS (0.2 mM) 2.5-fold. Similarly, the cytotoxicity of bleomycin (2 μM) increased from 22% to 62%, almost 3-fold, under these conditions.

### 1.3 Summary

Inhibitors that are selective for one DNA polymerase over another are uncommon. Covalent inhibitors, including those that react with lysine, are employed with increasing frequency to target proteins. Cuesta et al., 2019; Wan et al., 2020; Pettinger et al., 2017. Roughly 30% of marketed drugs are covalent in nature. Sutanto et al., 2020. Covalent inhibitors often lead to greater potency and longevity of effects, when they also are irreversible. Depending on the electrophilicity of the warhead, covalent inhibitors can enhance selectivity towards certain nucleophiles or residues they modify. To the best of current knowledge, the dioxobutane family of molecules, such as those presented here, are the only examples of molecules that irreversibly inhibit DNA polymerase β. The molecule described here (14) selectively inactivates Pol β. The corresponding pro-inhibitor (pro-14) selectively targets this enzyme in mouse embryonic fibroblasts. Although pro-14 it works synergistically with DNA damaging agents to kill mouse embryonic fibroblasts and HeLa cells, it is itself not highly toxic under the same conditions. This suggests that pro-14 will be a useful tool for studying the effects of Pol β inhibition in cells. Furthermore, the approach described here and elsewhere, Arian et al., 2014; Paul et al., 2017; Paul et al., 2018, for identifying Pol β inhibitors may be useful for targeting other polymerases.

### 1.4 General Methods

Modified oligonucleotides were synthesized on an Applied Biosystems Incorporated 394 oligonucleotide synthesizer. Oligonucleotide synthesis reagents including 5′-phosphorylation reagent (Solid CPR II), SIMA HEX (dichloro diphenyl fluorescein) phosphoramidite, THE abasic site analogue (dSpacer), TAMRA phosphoramidite, and BHQ phosphoramidite were purchased from Glen Research (Sterling, VA). Oligonucleotides containing only native nucleotides were purchased from Integrated DNA Technologies (Coralville, IA) and were purified by 20% denaturing polyacrylamide gel electrophoresis (PAGE). Oligonucleotides were characterized using a Bruker AutoFlex III Maldi-TOF/TOF system.

All chemicals were purchased from Sigma Aldrich, Fisher, or Alfa and were used without further purification. Small quantities of all library compounds were purchased from Sigma Aldrich but were from a variety of vendors (e.g., Enamine, CombiBlocks, ChemApex, and the like) Pol η was purchased from EnzyMax. Sybr Gold was purchased from ThermoFisher. Trypsin, dNTPs, terminal deoxynucleotide transferase, Klenow exo-, and T4 polynucleotide kinase were obtained from New England Biolabs. Radionuclides were from Perkin Elmer. Poly-Prep columns were from BioRad. C18-Sep-Pak cartridges were obtained from Waters. Zip-Tips were purchased from Millipore. Photolyses were carried out in a Rayonet photoreactor fitted with 16 lamps having a maximum output at 350 nm. Quantification of radiolabeled oligonucleotides was carried out using a Molecular Dynamics Phosphorimager 840 equipped with ImageQuant.

UPLC-MS/MS analyses were carried out on a Waters Acquity/Xevo-G2 UPLC-MS system equipped with an ACQUITY UPLC HSS T3 Column (100 Å, 1.8 μm, 2.1 mm×100 mm). Masses were obtained via deconvolution using MassLynx 4.2 software or BioPharmaLynx 1.3.2 software.

Well plates used for organic solvents and photolyses were obtained from VWR. Well plates used for fluorescence assays were obtained from Corning (CLS3825, 384 well plates, for homogenous luminescent and HTRF assays). Fluorescence data were collected on a Varian Cary Eclipse fluorescence spectrophotometer equipped with a well plate attachment. Fluorescence anisotropy measurements were conducted using an AVIV Biomedical Model ATF 107 spectrofluorometer at the Center for Molecular Biophysics at Johns Hopkins University.

The following items were generous gifts from colleagues. Plasmids for the 8 kDa and 31 kDa domains were from Dr. Sam Wilson, NIH. The Pol β domains were prepared using previously reported conditions. Prasad et al., 1993; Beard and Wilson, 1995. Pol λ plasmid was from Professor Zucai Suo, Florida State University. Pol λ was expressed and purified using previously reported conditions. Garcia-Diaz et al. 2000, Fiala et al., 2004.

Dulbecco's Modified Eagle Medium (DMEM) with high glucose was obtained from ThermoFisher. Antibiotic antimycotic solution (penicillin, streptomycin, and amphotericin B), and fetal bovine serum (FBS) were obtained from MilliporeSigma. PBS buffer was obtained from Quality Biological. Cells were counted using a BioRad TC20 cell counter. All small molecules synthesized were characterized using a Bruker Avance 400 MHz Spectrometer or a Varian Inova 800 MHz spectrometer at The Johns Hopkins University.

All Rr values provided in TLC information correspond to the product unless otherwise explicitly stated.

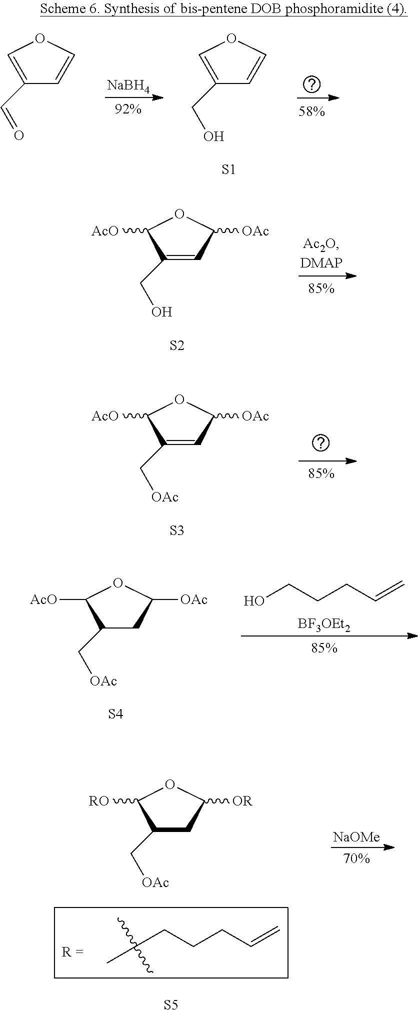

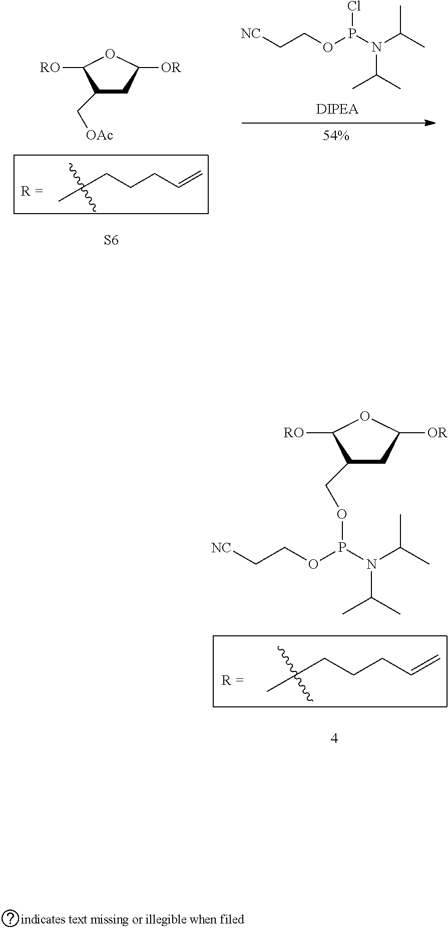

### Preparation of S1 (see Xu et al., 2015; Arian et al., 2014)

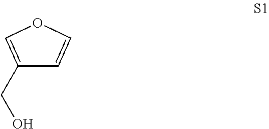

Commercially available 3-furaldehyde (4.38 mL, 5 g, 52.4 mmol, 1 eq) was added to a mixture of 10:1 Et₂O/H₂O (59 mL). The mixture was cooled to 0° C. and stirred. Small aliquots of NaBH4 (3.27 g, 86.4 mmol, 6.65 eq) were added to the mixture carefully over a period of 15 min. The reaction was stirred in air for 30 min. After 30 min, TLC (4:1 Hex/EtOAc, R_{f}=0.3) showed the complete conversion of the starting material (R_{f}=0.5, UV active, did not stain with PAA) to a slightly more polar spot that was not UV active and stained dark purple with PAA. The reaction was quenched with water until there was no more bubbling. The ether layer was washed with water (2×50 mL) and the combined aqueous layers were extracted with ether (6×40 mL). The final organic layer was washed with brine (1×50 mL), dried with MgSO4, and concentrated under vacuum at 0° C. to yield 4.714 g (91.7%) of a yellow liquid. The product was volatile, so the rotary evaporatory water bath was cooled to 0° C. during concentration. NMR data showed the crude product was pure. ¹H NMR (400 MHz, CDCl₃) δ 7.36 (s, 2H), 6.38 (s, 1H), 4.46 (d, J=4.9 Hz, 2H), 2.92 (d, J=4.9 Hz, 1H).

### Preparation of S2 (see Xu et al., 2015; Arian et al., 2014)

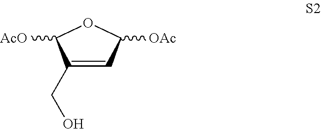

Without purification, S1 (474.6 mg, 4.84 mmol, 1 eq) was combined with Pb(OAc)₄ (3.22 g, 7.22 mmol, 1.5 eq). Glacial acetic acid (12.5 mL) was added to the flask, which was then flushed with Argon. The reaction was stirred at 25° C. for 21 h. When the reaction was complete by TLC (3:7 EtOAc/DCM, R_{f}=0.4, stained with PAA), AcOH was removed via vacuum. Ether (50 mL) was added to the resulting residue and the precipitate was triturated with ether and removed. The filtrate was concentrated under vacuum and purified by column chromatography (7:3 Hexanes/EtOAc) to give 518 mg (58%) of a 2:1 mixture of diastereomers (S2). ¹H NMR (400 MHz, CDCl₃) δ 6.90 (dd, J=1.0, 4.7 Hz, 1H), 6.69 (dd, J=1.0, 4.7 Hz, 1H), 6.15-6.05 (m, 1H), 4.30 (q, J=1.0 Hz, 2H), 2.19-2.07 (m, 6H).

### Preparation of S3

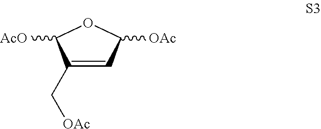

Compound S2 (150 mg, 0.685 mmol, 1 eq, 500 mM) and DMAP (8.6 mg, 0.07 mmol, 0.1 eq) were added to a flask. The contents were flushed with Argon and dissolved in DCM (1.25 mL). The mixture was cooled to 0° C. Acetic anhydride (0.33 mL, 3.5 mmol, 5 eq) was added slowly to the flask via syringe. Pyridine (0.68 mL, 8.56 mmol, 12.5 eq) was added dropwise to the solution via syringe. After 2 h, the reaction was confirmed complete by TLC (6:4 EtOAc/DCM, R_{f}=0.6, stained with PAA) and was quenched with sat. NaHCO₃ until the pH was neutral. The mixture was diluted with EtOAc (5 mL) and washed with water (2×5 mL). The aqueous layer was extracted with EtOAc (2×10 mL), the combined organic layers were washed with brine (1×30 mL), dried over Na₂SO₄, and concentrated under vacuum. The crude residue was purified by column chromatography (5:1 DCM/EtOAc) to yield 167 mg (85%) of S3. ¹H NMR (400 MHz, CDCl₃) δ 6.83 (s, 1H), 6.70-6.56 (m, 1H), 6.22-5.89 (m, 1H), 4.83-4.57 (m, 2H), 2.11-1.96 (m, 9H). ¹³C NMR (CDCl₃) 166.4, 152.3, 138.1, 111.6, 85.9, 85.8, 62.3, 38.1, 12.4, 9.2. ESI-TOF m/z calculated for C₁₁H₁₄O₇ (M+H)—258.0740, 258.0703 observed.

### Preparation of S4

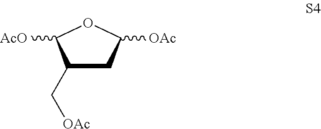

Compound S3 (150 mg, 0.69 mmol, 1 eq) was dissolved in EtOAc (17 mL). Rhodium on alumina catalyst (75.5 mg) was added to the pressure bottle equipped with a regulator. The vial was pressurized with H₂ to 70 psi, purged three times, and stirred at 25° C. for 2-4 h. After venting the pressure bottle, TLC (1:1 Hex/EtOAc, R_{f}=0.4, stained with PAA) showed the starting material was no longer present. When complete, the reaction mixture was passed through celite to remove the Rh catalyst. The filtrate was concentrated under vacuum to give 127.5 mg (85%) of pale, yellow compound S4. No purification was needed. ¹H NMR (400 MHz, CDCl₃) δ 6.25-6.15 (m, 2H), 4.06 (m, 1H), 4.03-3.91 (m, 1H), 2.71-2.56 (m, 1H), 2.38 (m, 1H), 2.04-1.88 (m, 9H), 1.83-1.67 (m, 1H). ¹³C NMR (CDCl₃) 170.7, 169.9, 169.5, 98.5, 96.4, 62.1, 41.6, 32.7, 21.5, 20.9, 20.7. ESI-TOF m/z calculated for C₁₁H₁₆O₇ (M+H)—260.0896, 260.0900 observed.

### Preparation of S5

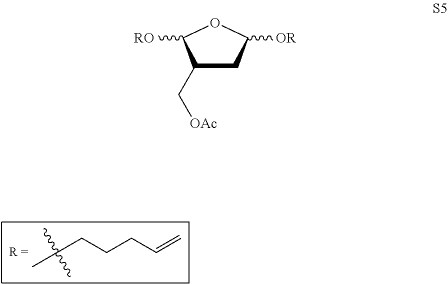

BF3·etherate was distilled from CaH₂ under vacuum and kept under Argon. Compound S4 (750 mg, 2.88 mmol, 1 eq, 160 mM) was dissolved in DCM (18 mL) and cooled to 0° C. 4-Pentenol (1.86 mL, 1.49 g, 17.3 mmol, 6 eq) was added to the reaction. BF3·etherate (8.14 mL, 1.67 M) was slowly added to the solution over a period of 15-20 min (until diluted to 28 mL, 600 mM). After 30 min, the reaction was incomplete when analyzed by TLC (7:3 Hex/Et₂O, stained with PAA) but additional products began to appear, so the reaction was quenched with sat. NaHCO₃ (5 mL), diluted with DCM (20 mL), and washed with sat. NaHCO₃ (1×15 mL). The aqueous layer was extracted with DCM (4×20 mL). The organic layer was washed with water (1×30 mL), brine (1×30 mL), and dried over Na₂SO₄. The residue was concentrated under vacuum and purified by column chromatography (8:2 hex/EtOAc) to give 228.6 mg (25.4%) of S5a and 519 mg (57.7%) of S5b (totaling 83%), which were each a mixture of diastereomers. S5a ¹H NMR (400 MHz, CDCl₃) δ 5.82-5.65 (m, 2H), 5.21-4.84 (m, 6H), 4.24-3.99 (m, 2H), 3.75-3.57 (m, 2H), 3.45-3.22 (m, 2H), 2.61 (m, 1H), 2.42-2.17 (m, 1H), 2.08-2.01 (m, 4H), 1.99 (d, J=9.5 Hz, 3H), 1.93-1.74 (m, 1H), 1.67-1.50 (m, 4H). S5b ¹H NMR (400 MHz, CDCl₃) δ 5.91-5.68 (m, 2H), 5.10-4.83 (m, 6H), 4.15-3.88 (m, 2H), 3.73 (m, 2H), 3.55-3.32 (m, 2H), 2.65 (ddd, J=5.5, 7.4, 13.5 Hz, 1H), 2.39 (s, 1H), 2.32-2.18 (m, 1H), 2.16-2.08 (m, 4H), 2.05 (s, 3H), 1.81 (ddd, J=5.5, 7.4, 13.5 Hz, 1H), 1.73-1.59 (m, 4H).

S5a and S5b ¹³C NMR (CDCl₃) 170.9, 170.8, 138.20, 138.18, 138.15, 114.8, 106.9, 106.2, 104.7, 104.2, 103.5, 103.2, 67.6, 67.5, 67.4, 67.3, 67.2, 67.0, 64.8, 64.5, 63.3, 43.8, 43.2, 40.7, 34.7, 34.2, 33.4, 30.4, 30.3, 29.9, 28.9, 28.8, 20.9, 20.8. ESI-TOF m/z calculated for C₁₇H₂₀O₅ (M+H)—312.1937, 312.1942 observed.

### Preparation of S6 (see Arian et al., 2014)

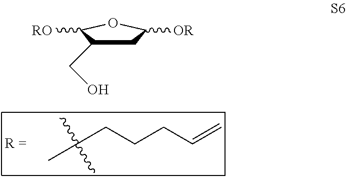

The diastereomerically enriched mixture (S5a or S5b) (82 mg, 0.26 mmol, 1 eq, 150 mM) was dissolved in MeOH (1.7 mL). Sodium methoxide stock solution (700 mM) was prepared by dissolving Na metal (122 mg, 5.3 mmol) in MeOH (7.5 mL). An aliquot of NaOMe (700 mM, 0.3 mL) was added to the reaction slowly (effectively diluting NaOMe to 100 mM). After 2 h, TLC (6:4 Hex/EtOAc, R_{f}=0.2, stained with PAA) confirmed the reaction was complete by the disappearance of S5. The reaction was quenched with a few drops of AcOH until neutral pH. The reaction was diluted with DCM (20 mL) and washed with H₂O (2×15 mL). The aqueous layer was extracted with DCM (4×20 mL) and the combined organic layers were washed with brine (1×30 mL) and dried over Na₂SO₄. The reaction was concentrated under vacuum to give 49.3 mg (70%) of S6. No purification was needed. ¹H NMR (400 MHz, CDCl₃) δ 5.91-5.65 (m, 2H), 5.23-4.88 (m, 6H), 3.93-3.71 (m, 2H), 3.72-3.61 (m, 2H), 3.49-3.31 (m, 2H), 2.63-2.46 (m, 1H), 2.38-2.23 (ddd, J=5.0, 9.5, 13.3 Hz, 1H), 2.26-2.13 (m, 1H), 2.13-2.03 (m, 4H), 1.82 (ddd, J=1.5, 7.8, 13.3 Hz, 1H), 1.65 (dddt, J=1.5, 5.0, 7.8, 9.5 Hz, 4H).

### Preparation of 4

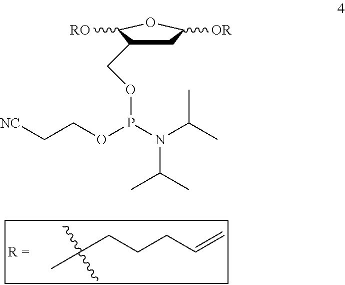

Compound S6 (58.2 mg, 0.22 mmol) was azeotropically dried with pyridine (2×0.5 mL). The reagent was cooled to 0° C. DIPEA (0.18 mL, 133 mg, 0.88 mmol, 4 eq) was added to the cold starting material and the reactants were dissolved in DCM (2.1 mL, 100 mM). 2-Cyanoethyl-N, N-diisopropylchlorophosphoramidite (0.06 mL, 60.4 mg, 0.26 mmol. 1.2 eq) was added and the cold mixture stirred with periodic monitoring by TLC (7:1 Hex/EtOAc, R_{f}=0.3, stained with PAA). After 2 h, TLC showed complete conversion to the phosphoramidite. The reaction was diluted with freshly distilled EtOAc (10 mL). The organic layer was washed with saturated bicarbonate solution (2×15 mL) and the aqueous layers were extracted with distilled EtOAc (2×20 mL). The combined organic layers were washed with brine (1×25 mL) and dried over Na₂SO₄. The organic layer was concentrated under vacuum and purified by column chromatography (7:1 distilled hexanes/distilled EtOAc) yielding 55.9 mg (54%) of 4. ¹H NMR (400 MHz, CDCl₃) δ 5.80 (dddt, J=4.1, 8.2, 13.5, 16.0 Hz, 2H), 5.18-4.93 (m, 6H), 3.86-3.72 (m, 2H), 3.64 (dtd, J=2.1, 6.0, 8.2 Hz, 2H), 3.59-3.51 (m, 2H), 3.34 (dddd, J=2.1, 4.1, 9.4, 16.0 Hz, 2H), 2.71-2.52 (m, 2H), 2.30 (ddd, J=6.0, 9.4, 13.5 Hz, 2H), 2.11-2.02 (m, 4H), 1.93-1.76 (m, 1H), 1.65-1.55 (m, 6H), 1.17 (m, 12H). ¹³C NMR (400 MHz, CDCl₃): δ 170.92, 170.91, 138.28, 138.25, 138.23, 114.75, 114.73, 114.69, 114.68, 114.64, 106.9, 105.7, 105.2, 104.5, 103.8, 102.6, 67.6, 67.3, 66.6, 64.5, 63.4, 44.8, 43.8, 43.2, 42.8, 34.7, 30.0, 30.3, 28.8, 20.9, 20.8. ³¹P NMR (400 MHz, CDCl₃) δ 147.87. ESI-TOF m/z calculated for C₂₄H₄₃N₂O₅P (M+H)—471.2910, 471.2887 observed.

### Preparation of 3 (see Handlon and Oppenheimer, 1998; Vanheusden et al., 2003)

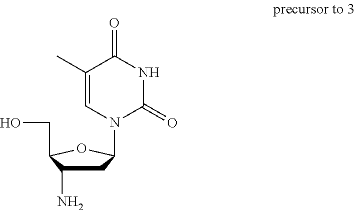

AZT (2.19, 8.2 mmol) was dissolved in 50% MeOH, 30% tBuOH, 20% H₂O (82 mL, 100 mM). Activated palladium on carbon (1.3 g, 60 wt %) was added and flushed with a hydrogen balloon three times. The reaction was continuously sparged with H₂ (1 atm). After 3 h, TLC (3% MeOH in DCM, UV active and stained with PAA) confirmed the starting material (R_{f}=0.8) converted to the product (R_{f}=0.02). The crude mixture was filtered through Celite and concentrated to yield 1.7 g (86%) of the 3′-amine precursor to 3. ¹H NMR (400 MHz, CD₃OD) δ7.87 (s, 1H), 6.18 (dd, J=2, 6.8 Hz, 1H), 3.86 (dd, J=2, 10 Hz, 1H), 3.77 (dd, J=3.2, 10 Hz, 1H), 3.70 (quint, J=3.2 Hz, 1H), 3.54 (q, J=6.8 Hz, 1H), 2.26 (m, 1H), 2.20 (m, 1H), 1.88 (s, 3H).

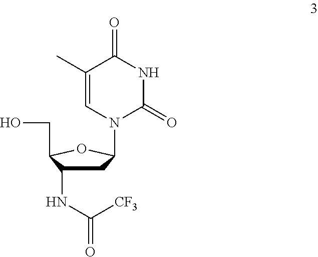

The crude mixture (1.72 g, 7.12 mmol) was dissolved in THF (25 mL, 280 mM). Triethylamine (14 mL, 106.8 mmol, 15 eq) and ethyl trifluoroacetate (8.4 mL, 71.3 mmol, 10 eq) were added to the flask to make a final concentration of 100 mM. The reaction was stirred for 4 h at 25° C. TLC (3% MeOH in DCM, R_{f}=0.5, UV active and stained by PAA) confirmed conversion to product. The reaction was concentrated and purified by column chromatography (1% MeOH in DCM to yield 2.29 g (93%) of 3. ¹H NMR (400 MHz, CD₃OD) δ 7.85 (s, 1H), 6.27 (t, J=6.4 Hz, 1H), 4.60 (q, J=6.4 Hz, 1H), 3.97 (quint, J=2.8 Hz, 1H), 3.85 (dd, J=2.8, 12 Hz, 1H), 3.73 (dd, J=2.8, 12 Hz, 1H), 2.40 (m, 2H), 1.89 (s, 3H). ¹³C NMR (400 MHz, CD₃OD) δ 166.2, 159.4, 158.9, 158.6, 152.2, 138.0, 118.6, 115.8, 111.5, 85.9, 85.6, 62.3, 50.7, 37.9, 12.3, 9.09. ESI-TOF m/z calculated for C₁₂H₁₄F₃N₃O₅ (M+H)—338.0886, 338.3413 observed.

### Preparation of 5

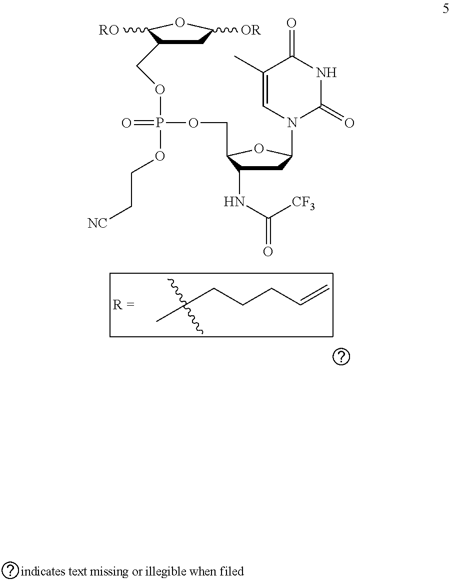

Phosphoramidite 4 (101 mg, 0.38 mmol, 1.2 eq) and 3 (172 mg, 0.32 mmol, 1 eq) were azeotropically dried together with toluene (2×1 mL). S-Ethyl tetrazole/MeCN (250 mM, 1.5 mL, 0.38 mmol, 1.2 eq) was added to the flask. After 4 h, TLC (1:1 EtOAc/Hex, R_{f}=0.6, UV active and stained with PAA) indicated that the majority of 55 was consumed. tBuOOH (500 mM, 200 μL, 1 mmol, 3 eq) was added and the reaction was stirred for 15-20 min. The reaction was concentrated under vacuum and column chromatography (2:1 EtOAc/Hex) yielded 131 mg (56%) of 5. ¹H NMR (400 MHz, CDCl₃) δ 10.36 (d, J=10.1 Hz, 1H), 8.86 (d, J=5 Hz, 1H), 7.46 (d, J=2 Hz, 1H), 6.38 (q, J=5 Hz, 1H), 5.74 (dsextet, J=2, 4 Hz, 2H), 5.10-4.90 (m, 6H), 4.51 (s, 1H), 4.34 (s, 2H), 4.25 (q, J=6 Hz, 2H), 4.16 (s, 2H), 4.06 (m, 1H), 3.63 (sextet, J=2 Hz, 2H), 3.33 (m, 2H), 2.75 (q, J=6 Hz, 3H), 2.44 (q, J=8 Hz, 2H), 2.28 (m, 1H), 2.15 (m, 1H), 2.01 (d, J=4 Hz, 4H), 1.96 (s, 3H), 1.58 (q, J=4 Hz, 4H). ¹³C NMR (400 MHz, CD₃OD) δ 166.23, 159.79, 152.15, 141.21, 139.40, 137.73, 129.01, 115.25, 113.27, 112.05, 106.87, 105.52, 104.85, 85.22, 84.82, 68.26, 67.38, 64.33, 61.54, 50.55, 48.65, 45.97, 37.24, 33.76, 31.45, 30.10, 27.94, 20.10, 15.31, 12.63. ³¹P NMR (400 MHz, CDCl₃) δ −2.14. ESI-TOF m/z calculated for C₃₀H₄₂F₃N₄O₁₁P (M+H)—723.2540.2717, 723.2303 observed.

### Preparation of 6

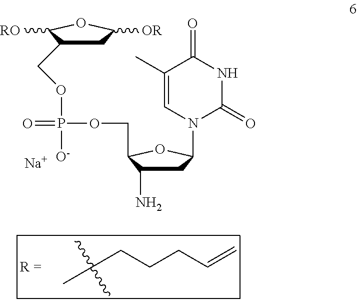

Compound 5 (115 mg, 0.15 mmol) was dissolved in concentrated aqueous ammonia (1.5 mL). The reaction was stirred at 0° C. for 4 h. After 4 h, TLC (20% MeOH in DCM, R_{f}=0.2, UV active and stained with KMnO4) confirmed the disappearance of starting material. The reaction was concentrated to yield 85 mg (100%) of 6. ¹H NMR (400 MHz, CD₃OD) δ 6.34 (t, J=5.8 Hz, 1H), 5.78 (m, 2H), 5.10-4.98 (m, 6H), 4.13 (s, 3H), 3.88 (m, 2H), 3.66 (m, 2H), 3.40 (q, J=5.8 Hz, 2H), 2.81 (s, 2H), 2.68 (t, J=7 Hz, 1H), 2.49 (m, 1H), 2.44 (m, 2H), 2.30 (m, 1H), 2.06 (t, J=7 Hz, 4H), 1.93 (s, 3H), 1.60 (quint, J=7 Hz, 4H). ¹³C NMR (400 MHz, CD₃OD): δ 164.92, 150.84, 138.03, 137.99, 136.57, 116.47, 113.84, 110.75, 105.95, 104.39, 103.54, 67.30, 66.66, 50.97, 35.82, 35.58, 30.04, 30.02, 28.72, 15.82, 14.19, 11.28. ³¹P NMR (400 MHz, CD₃OD) δ 0.07. ESI-TOF m/z calculated for C₂₅H₃₉N₃O₁₀P (M−H)—572.2379, 572.2399 observed.

### General Procedure for the Preparation of First-Generation Library

Amine scaffold 6 (100 nmol) was azeotropically dried with carboxylic acid (140 nmol, 1.4 eq) in pyridine (1×15 μL) using a Speed Vac concentrator in a 384-well microtiter plate (VWR). To each well, activating solution (5 μL; 28 mM HBTU and 28 mM HOBt in DMF), DIPEA (2 μL), and DMF (3 μL) were added. The final concentrations during reaction were: [6]=10 mM, [acid]=14 mM, [HBTU]=14 mM, [HOBt]=14 mM, 20% DIPEA in DMF.

The well plate was shaken at 25° C. overnight. Some wells were analyzed by ESI-MS to confirm coupling efficiency. The solutions were concentrated to dryness using a Speed Vac concentrator and the well plate was covered and stored at −80° C. Immediately before an assay, the amide was thawed, dissolved in DMF (4 μL, 25 mM). An aliquot (2 μL, 50 nmol) was mixed with NBS (8 μL, 15 mM, 2.4 eq, 40% H₂O in MeCN) at 0° C. for 9 min. The concentrations during reaction were: [SM]=5 mM, [NBS]=12 mM, 20% DMF, 30% H₂O in MeCN. After 9 min, Na₂S₂O₃ (5 μL, 200 mM) was added and reaction quenched on ice for 10 min. Samples were concentrated with a Speed Vac concentrator. Some samples were analyzed ESI-MS to confirm product formation.

### Preparation of 7

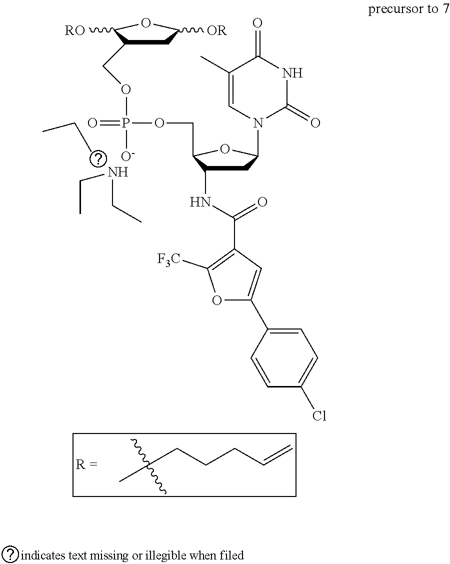

Scaffold 6 (17 mg, 0.03 mmol) and 5-(4-chlorophenyl)-3-(trifluoromethyl) furan-2-carboxylic acid (11 mg, 0.04 mmol, 1.4 eq) were azeotropically dried together with pyridine (2×1 mL). HBTU (14.0 mg, 0.037 mmol, 1.4 eq) and HOBt (5 mg, 0.037 mmol, 1.4 eq) were added to the starting materials. The contents were dissolved in DMF (1.04 mL) and DIPEA (260 μL). The reaction was stirred at 25° C. overnight. TLC (20% MeOH in DCM, R_{f}=0.3, UV active and stained with PAA) suggested 57 was consumed. The reaction was concentrated and purified by column chromatography (5% MeOH in DCM) to yield 21.2 mg (96%) of the precursor to 7. ¹H NMR (400 MHz, CD₃OD) δ 7.76 (d, J=6.8 Hz, 1H), 7.70 (d, J=7.6 Hz, 2H), 7.59 (d, J=6.8 Hz, 2H), 7.50 (d, J=7.6 Hz, 1H), 7.35 (quint, J=6.8 Hz, 4H), 6.37 (t, J=3.6 Hz, 1H), 5.78 (m, 2H), 5.00 (m, 6H), 4.68 (m, 1H), 4.15 (d, J=1.6 Hz, 1H), 4.10 (m, 2H), 3.90 (m, 2H), 3.66 (dd, J=1.6, 5.2 Hz, 2H), 3.21 (q, J=8 Hz, 6H), 2.49 (m, 1H), 2.40 (d, J=1.6 Hz, 2H), 2.30 (m, 1H), 2.07 (d, J=6.4 Hz, 4H), 1.97 (s, 3H), 1.90 (m, 1H), 1.60 (quint, J=6.4 Hz, 4H), 1.32 (t, J=8 Hz, 9H). ¹³C NMR (400 MHz, CDCl₃) δ 169.9., 169.7, 163.8, 150.2, 136.2, 134.9, 128.6, 123.9, 116.6, 111.43, 111.40, 98.45, 98.42, 96.1, 87.3, 87.2, 86.2, 86.1, 80.9, 80.8, 67.3, 66.3, 66.2, 62.5, 62.45, 62.43, 42.9, 42.8, 42.7, 36.1, 32.3, 32.2, 21.1, 19.72, 19.67, 12.4. ³¹P NMR (400 MHz, CD₃OD) δ −2.4, −3.0. ESI-TOF m/z calculated for C₃₇H₄₄ClF₃N₃O₁₂P (M−H)—844.2303, 844.2221 observed. 8273 (50% MeCN in H₂O)=2.0×104 M⁻¹ cm⁻¹.

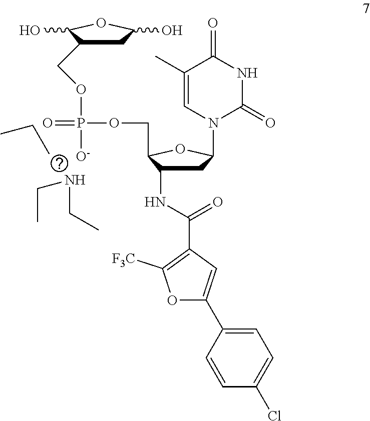

The pentenoyl protected inhibitor was stored as a 25 mM stock solution in DMF at −20° C. An aliquot of the starting material (2 μL, 50 nmol) was placed in a microtiter well plate and cooled to 4° C. Cold H₂O (3 μL) was added to the well. NBS in MeCN (25 mM, 5 μL) was added and the well plate was shaken at 4° C. for 9 min. After 9 min, the reaction was quenched by an equal volume of Na₂S₂O₃ (200 mM in H₂O, 10 μL) and the plate was shaken for additional 5-10 min. The reaction was concentrated by speed vacuum, redissolved in 1:1 MeCN/H₂O and used directly. Reaction Conditions: [SM]=5 mM, [NBS]=12.5 mM. 30% H₂O, 20% DMF, 50% MeCN Quenching Conditions: [SM]=2.5 mM, [NBS]=6.25 mM, [Na₂S₂O₃]=100 mM, 65% H₂O, 10% DMF, 25% MeCN. ESI-MS m/z calculated for C₂₇H₂₇ClF₃N₃O₁₂P (M−H)—708.09, 708.20 observed.

### Preparation of 8 (see Vernekar et al., 2015)

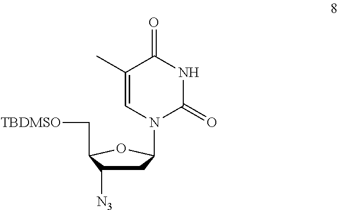

AZT (587 mg, 2.19 mmol, 1 eq) was azeotropically dried twice with pyridine (2×1 mL). Imidazole (604 mg, 8.76 mmol. 4 eq) and TBDMSCl (672 mg. 4.38 mmol, 2 eq) were added and dissolved in DMF (4.5 mL, 500 mM). The reaction was heated to 50° C. overnight. When confirmed complete by TLC (5% MeOH in DCM, R_{f}=0.5, UV active and stained with PAA), the reaction was cooled and diluted with EtOAc (10 mL). The organic layer was washed with sat. NH₄Cl (3×15 mL) and brine (2×15 mL). The combined aqueous layers were extracted using EtOAc (2×50 mL). The combined organic layers were dried over Na₂SO₄ and concentrated under vacuum. The residue was purified by column chromatography (2:1 Hex/EtOAc) yielding 825 mg (95%) of 8. ¹H NMR (400 MHz, CDCl₃) δ 8.72 (s, 1H), 7.43 (s, 1H), 6.22 (t, J=6.8 Hz, 1H), 4.24 (q, J=3.2 Hz, 1H), 3.96 (d, J=3.2 Hz, 1H), 3.94 (dd, J=2.8, 14 Hz, 1H), 3.79 (dd, J=2.8, 14 Hz, 1H), 2.44 (m, 1H), 2.22 (m, 1H), 1.91 (s, 3H), 0.93 (s, 9H), 0.13 (s, 6H). ¹³C NMR (400 MHz, CDCl₃) δ 134.9, 111.0, 84.5, 84.4, 62.9, 60.5, 37.9, 25.9, 25.7, 25.6, 18.3, 12.5, −5.4, −5.5.

### Preparation of 9

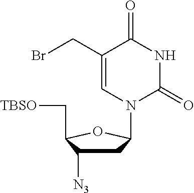

Compound 8 (982.8 mg, 2.58 mmol, 1 eq) was mixed with NBS (366 mg, 2.06 mmol, 0.8 eq) and dissolved in distilled benzene (13 mL, 200 mM). The mixture was sparged with Ar for 20 min. The reaction was activated by sun lamp. The reaction was stirred for 1 h while the mixture turned red. When TLC (1:1 Hex/EtOAc, UV active and stained with iodine) showed a mixture of starting material and one new spot, a second aliquot of 0.8 eq NBS was added to the reaction. After a total of 2 h and 2×0.8 eq NBS added, the SM was consumed to yield one major spot. The mixture was immediately filtered through a glass frit and diluted with DCM (10 mL) and H₂O (20 mL). The aqueous layer was extracted with DCM (2×20 mL) and the combined organic layers were washed with sat. bicarbonate solution (1×40 mL), brine (1×40 mL), dried over Na₂SO₄, and concentrated under vacuum to give 853.6 mg (72%) a yellow crude residue. ¹H NMR (400 MHz, CDCl₃) δ 9.67 (s, 1H), 7.84 (s, 1H), 6.17 (t, J=6.4 Hz, 1H), 4.24 (m, 2H), 4.20 (m, 1H), 3.98 (m, 1H), 3.96 (dd, J=2.8, 11.6 Hz, 1H), 3.80 (dd, J=2.8, 11.6 Hz, 1H), 2.50 (m, 1H), 2.25 (m, 1H), 0.94 (s, 9H), 0.14 (s, 6H).

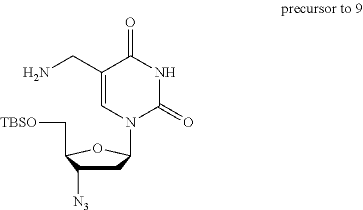

The crude bromide (853.2 mg, 1.85 mmol) was dissolved in EtOH (13 mL). Concentrated aqueous ammonia (23 mL, 15.7 M, 47.1 mmol, 25 eq) was added to the flask. The reaction was stirred at 25° C. for 1.5 h. After 1.5 h, TLC (10% MeOH in DCM) indicated the formation of product (R_{f}=0.3). The reaction was concentrated and purified by column chromatography (DCM->3% MeOH in DCM) to yield 339 mg (43%) of the amine. ¹H NMR (400 MHz, CDCl₃) δ 7.78 (s, 1H), 6.06 (t, J=6.4 Hz, 1H), 4.22 (m, 1H), 3.88 (q, J=4.4 Hz, 1H), 3.82 (d, J=4.4 Hz, 1H), 3.78 (d, J=4.4 Hz, 1H), 3.73 (s, 2H), 2.35 (t, J=6.4 Hz, 2H), 0.85 (s, 9H), 0.05 (s, 6H). ¹³C NMR (400 MHz, CDCl₃) δ 164.3, 150.3, 140.2, 109.2, 85.1, 84.6, 62.9, 60.6, 50.1, 37.3, 25.8, 22.4, 18.3, −5.4, −5.5. ESI-TOF m/z calculated for C₁₆H₂₈N₆O₄Si (M+H)—397.1941 calculated, 397.2012 observed.

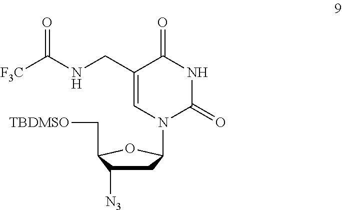

The second intermediate (313.5 mg, 0.791 mmol) was dissolved in THF (5.2 mL, 280 mM). Triethylamine (1.6 mL, 11.8 mmol, 15 eq) and ethyl trifluoroacetate (0.95 mL, 7.91 mmol, 10 eq) were added to the flask to make the final concentration 100 mM. The reaction was stirred for 4 h at 25° C. TLC (3% MeOH in DCM, R_{f}=0.5, UV active and stained by PAA) confirmed conversion to product. The reaction was concentrated and carried further to the next step without further purification, yielding 500 mg of 66 (91%). ¹H NMR (400 MHz, CDCl₃) δ 10.17 (s, 1H), 7.91 (s, 1H), 7.80 (s, 1H), 6.13 (t, J=6.4 Hz, 1H), 4.23 (q, J=4 Hz, 1H), 4.20 (s, 2H), 4.00 (q, J=4 Hz, 1H), 3.91 (dd, J=3.6, 11.6 Hz, 1H), 3.84 (dd, J=3.6, 11.6 Hz, 1H), 2.49 (m, 1H), 2.24 (m, 1H), 0.91 (s, 9H), 0.13 (s, 6H). ¹³C NMR (400 MHz, CDCl₃) δ 162.9, 161.3, 160.9, 149.9, 138.7, 117.8, 114.9, 109.2, 84.8, 84.6, 60.8, 59.9, 53.2, 49.2, 45.5, 37.4, 35.7, 7.9. ESI-TOF m/z calculated for C₁₈H₂₇F₃N₆O₅Pi (M+H)—493.1764, 493.1716 observed.

### Preparation of 10

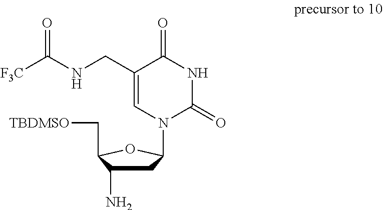

Compound 9 (22.5 mg, 0.05 mmol) was dissolved in 50% MeOH, 20% H₂O, 30% tBuOH (1 mL). Activated Pd/C (14.1 mg, 60 wt %) was added to the solution. The reaction was continuously sparged with H₂ (1 atm). After 1 h, TLC (5% MeOH in DCM, UV active and stained with PAA, R_{f}=0.35) suggested the reaction was complete. The mixture was passed through a Celite column and concentrated to 17 mg (82%) of crude amine, which was carried forward without further purification. ¹H NMR (400 MHz, CD₃OD) δ 7.83 (s, 1H), 6.18 (t, J=6.4 Hz, 1H), 4.12 (s, 2H), 3.93 (dd, J=4, 11.2 Hz, 1H), 3.88 (dd, J=4, 11.2 Hz, 1H), 3.77 (dt, J=1.2, 4 Hz, 1H), 3.50 (q, J=6.4 Hz, 1H), 2.25 (m, 2H), 0.93 (s, 9H), 0.12 (d, J=1.2 Hz, 6H).

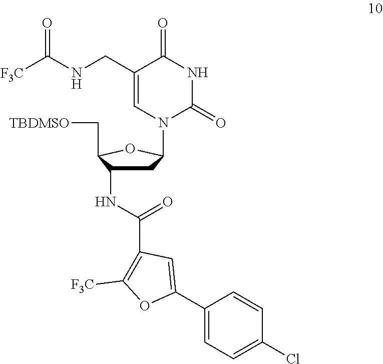

Crude amine (17 mg, 0.033 mmol) and 5-(4-chlorophenyl)-3-(trifluoromethyl) furan-2-carboxylic acid (14.2 mg, 0.05 mmol, 1.4 eq) were azeotropically dried together with pyridine (2×1 mL). HBTU (17.8 mg, 0.05 mmol, 1.4 eq) and HOBt (6.3 mg, 0.05 mmol, 1.4 eq) were added to the starting materials. The contents were dissolved in DMF (528 μL) and DIPEA (132 μL). The reaction was stirred at 25° C. overnight. TLC (10% MeOH in DCM. R_{f}=0.4, UV active and stained with PAA) suggested the starting material was gone. The reaction was concentrated and purified by column chromatography (1:1 Hex/EtOAc->5% MeOH, 1:1 Hex/EtOAc) to yield 18.1 mg (75%) of 10. ¹H NMR (400 MHz, CDCl₃) δ 8.23 (s, 1H), 8.10 (d, J=6.4 Hz, 1H), 7.86 (s, 1H), 7.75 (d, J=8.2 Hz, 2H), 7.63 (d, J=8.2 Hz, 2H), 6.09 (t, J=6.4 Hz, 1H), 4.59 (t, J=4 Hz, 1H), 4.12 (t, J=4 Hz, 2H), 3.88 (m, 2H), 2.43 (m, 1H), 2.25 (m, 1H), 0.82 (s, 9H), 0.04 (s, 6H). ¹³C NMR (400 MHz, CDCl₃) δ 162.7, 160.8, 150.1, 134.9, 129.0, 126.5, 125.8, 125.6, 120.1, 117.3, 110.7, 108.9, 105.9, 85.6, 54.7, 38.4, 36.5, 31.4, 25.7, 18.2, 16.9, −5.6. −5.7. ESI-TOF m/z calculated for C₃₀H₃₃ClF₆N₄O₇Si (M+H)—739.1711, 739.1784 observed.

### Preparation of S7

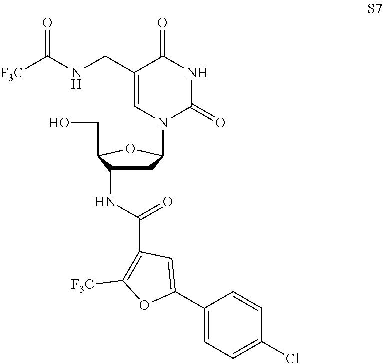

Compound 10 (135 mg, 0.19 mmol) was dissolved in THF (3.6 mL). TEA·3HF (74 mg, 0.46 mmol, 0.08 mL, 2.5 eq) was added to the flask. The reaction was stirred at 25° C. overnight. TLC (5% MeOH in DCM, R_{f}=0.5, UV active and stained with PAA) confirmed conversion to product. The reaction was concentrated and purified by column chromatography (DCM->2% MeOH) to yield 100 mg (86%) of S7. H NMR (400 MHz, CDCl₃) δ 8.28 (t, J=6.9, 1H), 7.79 (m, 3H), 7.74 (dd, J=1.6, 6.9 Hz, 1H), 7.57 (dd, J=1.6, 8 Hz, 1H), 7.23 (d, J=8 Hz, 1H), 7.19 (m, 2H), 6.06 (q, J=4.4 Hz, 1H), 4.54 (t J=7.6 Hz, 1H), 4.32 (d, J=7.6 Hz, 1H), 4.21 (dd, J=5.6, 14.8 Hz, 1H), 4.15 (dd, J=5.6, 14.8 Hz, 1H), 3.84 (d, J=10.8 Hz, 1H), 3.74 (d, J=10.8 Hz, 1H), 2.30 (m, 2H). ¹³C NMR (400 MHz, CDCl₃) δ 163.2, 160.8, 157.2, 153.0, 149.9, 134.1, 128.6, 127.4, 126.1, 124.4, 123.9, 120.2, 117.9, 110.9, 108.6, 107.4, 83.9, 50.1, 46.1, 8.31, 8.29, 8.26, 8.24. ESI-TOF m/z calculated for C₂₄H₁₉ClF₃N₄O₇ (M+H)—625.0846, 625.0844 observed.

### Preparation of 12

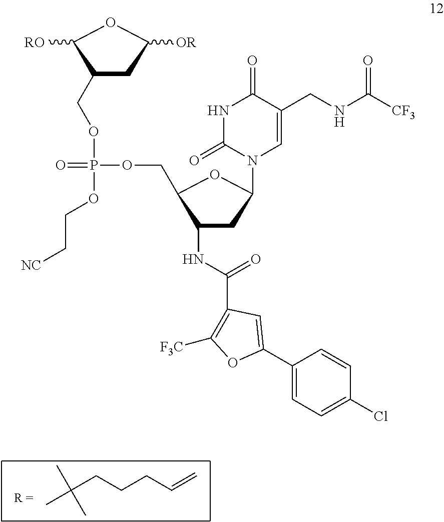

Compounds S7 (61 mg, 0.08 mmol, 1.2 eq) and 4 (32 mg, 0.07 mmol, 1 eq) were azeotropically dried with toluene (2×0.5 mL). S-Ethyl tetrazole/MeCN (250 mM, 1 mL, 0.24 mmol, 1.2 eq) was added to the reaction flask. After 3 h, TLC (2:1 EtOAc/Hex, R_{f}=0.5, UV active and stained with PAA) indicated that the majority of S7 was consumed. tBuOOH (500 mM, 18 mg, 40 μL, 0.20 mmol, 3 eq) was added for 15-20 min. The reaction was concentrated and column chromatography (2:1 EtOAc/Hex) yielded 61 mg (90%) of 12. ¹H NMR (400 MHz, CDCl₃) δ 7.87 (d, J=5.9, 1H), 7.56 (d, J=8.4 Hz, 2H), 7.35 (d, J=8.4 Hz, 2H), 7.07 (d, J=3.6 Hz, 1H), 6.22 (t, J=5.9, 1H), 5.78 (m, 2H), 4.99 (m, 6H), 4.75 (m, 1H), 4.40 (m, 3H), 4.30 (m, 4H), 4.28 (m, 2H), 3.88 (m, 2H), 3.61 (m, 2H), 3.38 (m, 2H), 2.78 (quint, J=3.6 Hz, 2H), 2.63 (m, 1H), 2.52 (m, 2H), 2.45 (m, 1H), 2.08 (m, 4H), 1.88 (m, 1H), 1.62 (m, 4H). ¹³C NMR (400 MHz, CDCl₃) δ 137.9, 135.1, 129.0, 126.5, 125.6, 114.6, 114.5, 66.6, 65.8, 30.1, 30.0, 28.6, 269, 26.1, 14.6. ³¹P NMR (400 MHz, CDCl₃) δ 7.8, −0.9, −1.0, −2.57, −2.62, −2.63, −2.65, −2.67, −2.69, −2.71, −3.06, −3.1. ¹⁹F NMR (300 MHz, CDCl₃) δ −64.02, −64.03, −64.1, −78.12, −78.13 −78.2. ESI-TOF m/z calculated for C₄₂H₄₇ClF₆N₅O₁₃P (M+H)—1010.2501, 1010.2183 observed.

### Preparation of 13

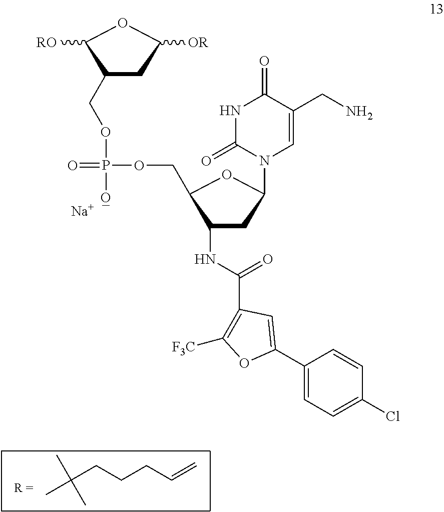

Compound 12 (8 mg, 0.008 mmol) was dissolved in concentrated aqueous ammonia (160 μL). The reaction was capped and stirred at 25° C. After 5 h, TLC (15% MeOH in DCM, R_{f}=0.1, UV active and stained with PAA) confirmed the disappearance of starting material and formation of a new spot. The reaction was concentrated and purified by column chromatography (5% MeOH in DCM->20% MeOH in DCM) to yield 9.3 mg (73%) of 13. The product was passed through a Dowex (Na+) column. ¹H NMR (400 MHz, CD₃OD) δ 8.37 (s, 1H), 7.76 (d, J=11.6 Hz, 2H), 7.68 (q, J=5.2 Hz, 1H), 7.50 (d, J=11.6 Hz, 2H), 7.24 (m, 3H), 6.28 (t, J=5.2 Hz, 1H), 5.80 (m, 2H), 4.95 (m, 6H), 4.78 (q, J=8.6 Hz, 1H), 4.26 (m, 2H), 4.08 (m, 2H), 3.83 (m, 4H), 3.63 (m, 2H), 2.53 (q, J=8.6 Hz, 2H), 2.47 (m, 2H), 2.09 (m, 4H), 1.60 (m, 4H). ¹³C NMR (400 MHz, CD₃OD) δ 166.3, 164.8, 139.4, 137.9, 136.3, 130.3, 128.4, 128.3, 127.0, 115.0, 114.98, 111.3, 108.1, 106.0, 68.5, 68.2, 62.2, 55.7, 43.7, 36.8, 35.1, 31.5, 31.4, 31.39, 31.35, 30.03, 30.00, 19.2, 18.6, 17.2, 13.0, 12.5. ³¹P NMR (400 MHz, CD₃CN) δ −2.3. ESI-TOF m/z calculated for C₃₇H₄₅ClF₃N₄O₁₂P (M−H)—859.2412, 859.2277 observed.

### General Procedure for the Preparation of Second-Generation Library

Amine scaffold 13 (100 nmol) was azeotropically dried with carboxylic acid (120 nmol, 1.2 eq) in pyridine (1×15 μL) using a Speed Vac concentrator in a 384-well microtiter plate (VWR). To each well, activating solution (5 μL; 24 mM HBTU and 24 mM HOBt in DMF), DIPEA (2 μL), and DMF (3 μL) were added. The final concentrations during reaction were: [13]=10 mM, [acid]=12 mM, [HBTU]=12 mM, [HOBt]=12 mM, 20% DIPEA in DMF. The well plate was shaken at 25° C. overnight. Some wells were analyzed by ESI-MS to confirm coupling efficiency. The solutions were concentrated to dryness using a Speed Vac concentrator and the well plate was covered and stored at −80° C. Immediately before an assay, the amide was thawed, dissolved in DMF (4 μL, 25 mM). An aliquot (2 μL, 50 nmol)

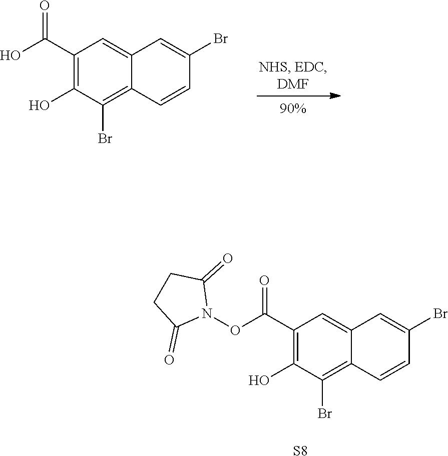 [SMILES: O=C(O)c1cc2cc(Br)ccc2c(Br)c1O ; O=C(ON1C(=O)CCC1=O)c1cc2cc(Br)ccc2c(Br)c1O ; "S8"]

was mixed with NBS (8 μL, 15 mM, 2.4 eq, 40% H₂O in MeCN) at 0° C. for 9 min. The concentrations during reaction were: [SM]=5 mM, [NBS]=12 mM, 20% DMF, 30% H₂O in MeCN. After 9 min, Na₂S₂O₃ (5 μL, 200 mM) was added and reaction quenched on ice for 10 min. Samples were concentrated to dryness with a Speed Vac concentrator. Some samples were analyzed ESI-MS to confirm product formation.

### Preparation of S8

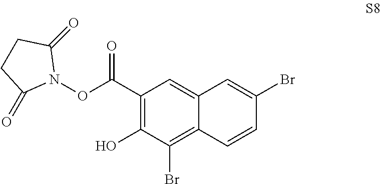 [SMILES: O=C(ON1C(=O)CCC1=O)c1cc2cc(Br)ccc2c(Br)c1O]

Commercially available 1,6-dibromo-3-hydroxy-2-naphthoic acid (102 mg, 0.3 mmol) was azeotropically dried in pyridine (2×1 mL). NHS (54 mg, 0.45 mmol, 1.5 eq), and EDC (86 mg, 0.45 mmol, 1.5 eq) were added to the flask and the reagents were dissolved in DMF and stirred overnight at 25° C. After 16 h, TLC (2% MeOH in DCM, R_{f}=0.8, UV active) suggested the SM was completely converted so the reaction was concentrated. The product was purified by column chromatography (DCM->1% MeOH in DCM) to yield 119 mg (90%) of NHS ester (S8). ¹H NMR (400 MHz, CDCl₃) δ 9.79 (s, 1H), 8.60 (s, 1H), 8.09 (d, J=8.8 Hz, 1H), 8.01 (s, 1H), 7.75 (d, J=8.8 Hz, 1H), 2.97 (s, 4H). ¹³C NMR (400 MHz, CDCl₃) δ 169.6, 162.5, 153.4, 134.6, 133.6, 131.2, 130.6, 128.2, 127.7, 118.3, 115.0, 107.2, 53.4, 53.1, 36.4, 31.4, 29.6. ESI-TOF m/z calculated for C₁₅H₉NO₅ (M+H)—441.8847, 441.8807 observed.

### Preparation of 14

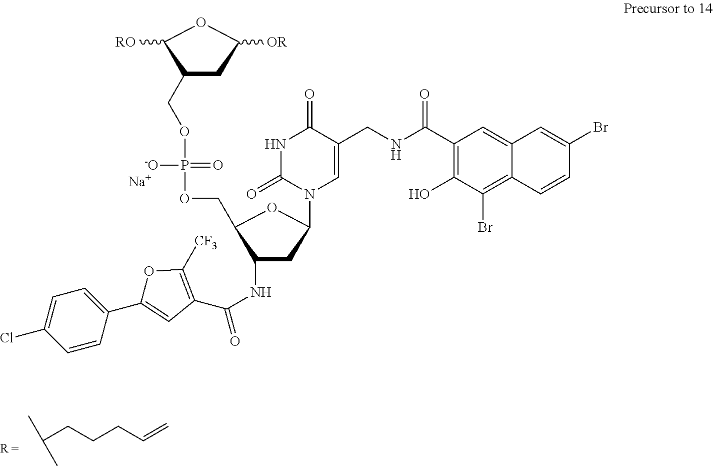

Scaffold 13 (14 mg, 0.016 mmol) and 15 (11 mg, 0.024 mmol, 1.5 eq) were azeotropically dried together with pyridine (2×1 mL). The contents were dissolved in DMF (350 μL) and DIPEA (5 μL). The reaction was stirred at 25° C. overnight. TLC (20% MeOH in DCM) indicated formation of product (R_{f}=0.3, UV active and stained with PAA). The reaction was concentrated and purified by column chromatography (2:1 Hex/EtOAc->1:1 Hex/EtOAc->1:1 Hex/EtOAc, 5% MeOH) to yield 12.2 mg (45%) of the precursor to 14. The product was passed through a Dowex (Na+) column. ¹H NMR (400 MHz, CD₃OD) δ 8.38 (s, 2H), 8.02 (s, 3H), 7.98 (d, J=7.2 Hz, 2H), 7.74 (d, J=8.8 Hz, 1H), 7.61 (d, J=7.2, 2H), 7.48 (d, J=8.8 Hz, 1H), 7.24 (m, 1H), 6.30 (s, 1H), 5.82 (m, 2H), 4.99 (m, 6H), 4.73 (s, 1H), 4.44 (s, 1H), 4.22 (s, 1H), 4.1 (m, 1H), 3.73 (m, 3H), 3.66 (m, 2H), 3.41 (m, 2H), 2.53 (m, 1H), 2.48 (m, 2H), 2.12 (q, J=7.2 Hz, 4H), 2.05 (m, 2H), 1.63 (q, J=7.2 Hz, 4H). ¹³C NMR (600 MHz, CD₃OD) δ 173.8, 172.4, 169.0, 163.8, 161.7, 155.7, 154.3, 143.8, 139.6, 138.1, 135.0, 133.9, 132.2, 131.2, 130.7, 129.8, 128.5, 127.1, 126.7, 125.7, 122.4, 116.3, 113.7, 106.5, 106.1, 105.6, 104.5, 85.6, 83.6, 45.4, 35.3, 31.7, 30.1, 29.4, 28.7, 24.8, 22.3, 13.0. ³¹P NMR (400 MHz, CD₃OD) δ 4.8, 1.7, −0.1, −0.2. ESI-TOF m/z calculated for C₄₈H₄₉ClF₃N₄O₁₄P (M−H)—1185.0990, 1185.0579 observed. e275 (50% MeCN in H₂O)=1.96×104 M⁻¹ cm⁻¹, e370=3.81×103 M⁻¹ cm⁻¹.

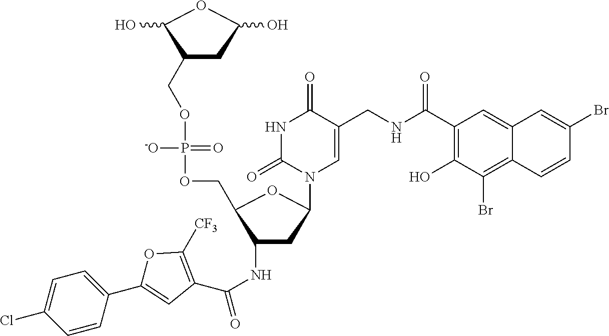

The starting material was stored as a 25 mM stock solution in DMF at −20° C. An aliquot of the starting material (2 μL, 50 nmol) was placed in a microtiter well plate and cooled to 4° C. Cold H₂O (3 μL) and NBS in MeCN (25 mM, 5 L) was added and the well plate was shaken at 4° C. for 9 min. After 9 min, the reaction was quenched by an equal volume of Na₂S203 (200 mM in H₂O) and well plate was shaken for additional 5-10 min. The reaction was concentrated by speed vacuum, redissolved in 1:1 MeCN/H₂O and used directly. Reaction Conditions: [SM]=5 mM, [NBS]=12.5 mM, 30% H₂O, 20% DMF, 50% MeCN Quenching Conditions: [SM]=2.5 mM, [NBS]=6.25 mM, [Na₂S203]=100 mM, 65% H₂O, 10% DMF, 25% MeCN. ¹H NMR (800 MHz, CD₃CN) S 9.31 (s, 1H), 8.46 (s, 1H), 8.00 (m, 1H), 7.91 (d, J=8 Hz, 1H), 7.75 (d, J=8 Hz, 2H), 7.71 (s, 1H), 7.68 (m, 1H), 7.64 (m, 1H), 7.53 (d, J=8 Hz, 2H), 7.50 (m, 1H), 7.37 (s, 1H), 7.32 (m, 2H), 7.11 (s, 1H), 6.32 (t, J=8 Hz, 1H), 4.93 (d, J=8 Hz, 1H), 4.78 (m, 1H), 4.67 (m, 1H), 4.48 (s, 1H), 4.37 (s, 1H), 4.22 (m, 1H), 4.17 (m, 1H), 4.05 (m, 1H), 3.91 (s, 1H), 3.91 (s, 1H), 3.80 (s, 1H), 3.69 (m, 3H), 3.59 (m, 2H). 3.43 (s, 1H), 2.95 (s, 2H), 2.47 (m, 3H), 2.28 (m, 3H). ESI-TOF m/z calculated for C₄₈H₄₉ClF₃N₄O₁₄P (M+H)—1049.9738, 1049.9699 observed.

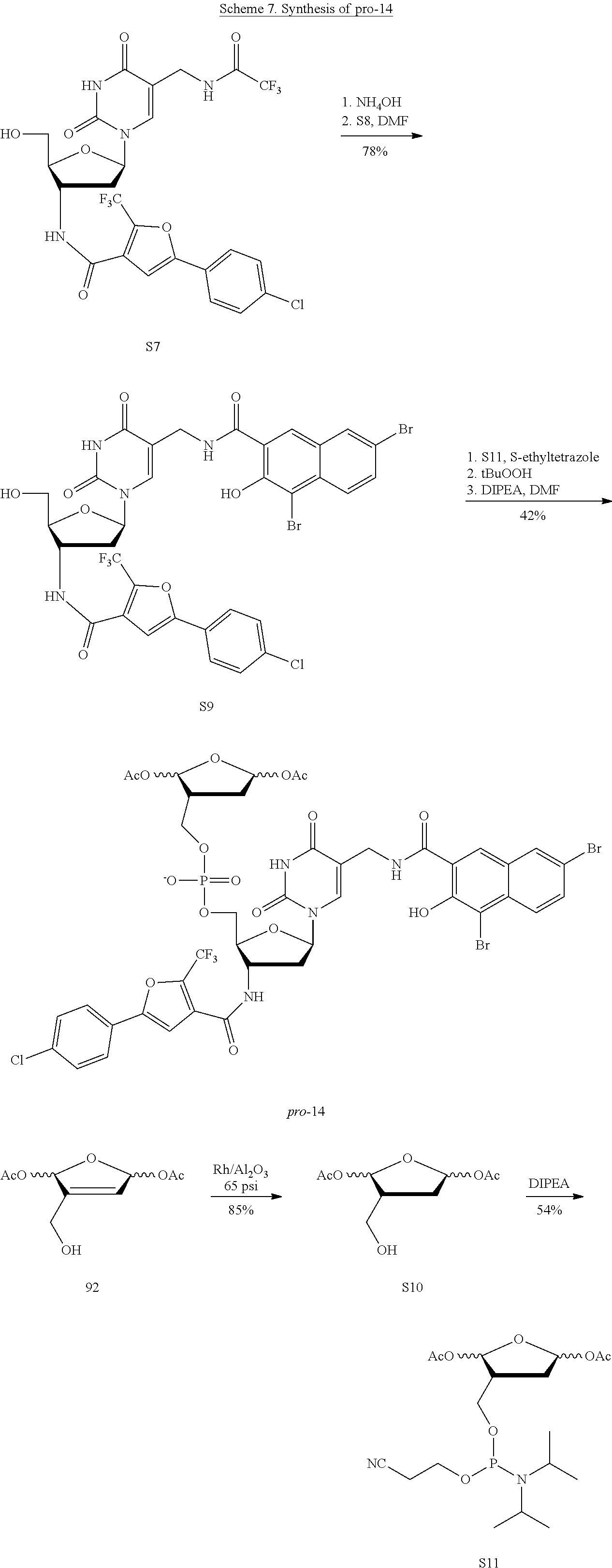

### Preparation of S9

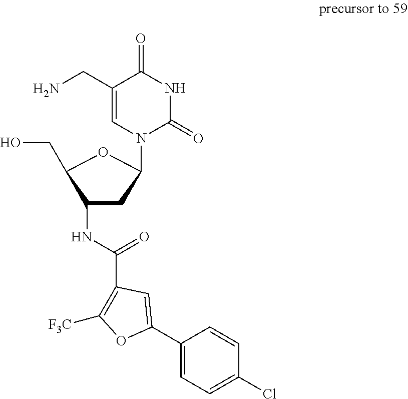 [SMILES: NCc1cn([C@H]2C[C@@H](NC(=O)c3cc(-c4ccc(Cl)cc4)oc3C(F)(F)F)[C@@H](CO)O2)c(=O)[nH]c1=O]

Compound S7 (50 mg, 0.08 mmol) was dissolved in concentrated aqueous ammonia (0.8 mL). The reaction was capped and stirred at 25° C. After 5 h, TLC (5% MeOH in DCM, R_{f}=0.2, UV active) confirmed the disappearance of starting material and formation of a new, more polar spot. The reaction was concentrated to yield 45 mg of crude amide.

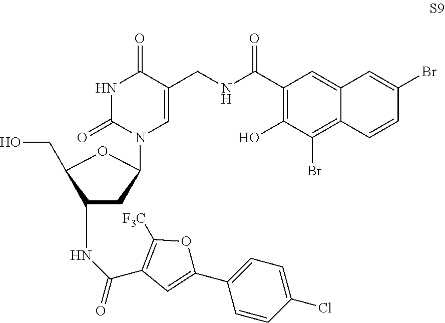 [SMILES: O=C(N[C@@H]1C[C@H](n2cc(CNC(=O)c3cc4cc(Br)ccc4c(Br)c3O)c(=O)[nH]c2=O)O[C@@H]1CO)c1cc(-c2ccc(Cl)cc2)oc1C(F)(F)F]

The crude amide (45 mg, 0.08 mmol) and NHS ester S8 (36 mg, 0.09 mmol. 1.1 eq) were azeotropically dried together in pyridine (2×0.5 mL). The contents were dissolved in DMF (1.6 mL). The reaction was stirred at 25° C. overnight. TLC (1:1 EtOAc/Hex, 2% MeOH, UV active, R_{f}=0.2) indicated the starting materials were converted to product. The reaction was concentrated and purified by column chromatography (2:1 Hex/EtOAc->1:1 Hex/EtOAc->1:1 Hex/EtOAc->2% MeOH->1:1 Hex/EtOAc, 5% MeOH) to yield 53 mg (78%) of S9. H NMR (400 MHz, CD₃OD) δ 8.56 (s, 3H), 8.34 (s, 1H), 8.26 (s, 1H), 8.04 (m, 3H), 7.94 (d, J=8 Hz, 1H), 7.86 (d, J=8 Hz, 1H), 7.21 (s, 1H), 6.30 (t, J=6.8 Hz, 1H), 4.69 (m, 1H), 4.34 (d, J=3.2 Hz, 2H), 4.03 (d, J=2.8 Hz, 1H), 3.87 (dd, J=3.2, 13.9 Hz, 1H), 3.77 (dd, J=2.8, 13.9 Hz, 1H), 2.45 (m, 2H). ¹³C NMR (600 MHz, CD₃OD) δ 199.3, 193.6, 193.1, 163.4, 161.7, 159.9, 157.7, 150.4, 142.3, 141.9, 140.0, 139.2, 138.9, 135.1, 129.0, 128.9, 126.7, 125.9, 125.2, 121.1, 119.6, 118.0, 117.7, 116.9, 110.3, 109.3, 107.3, 105.9, 85.7, 81.5, 64.6, 57.4, 50.3, 41.4, 37.5, 36.4, 36.0, 29.3, 25.1, 7.8. ESI-TOF m/z calculated for C₃₃H₂₄Br₂ClF₃N₄O₈ (M+H)—854.9602, 854.9636 observed.

### Preparation of S10 (see Paul et al., 2017)

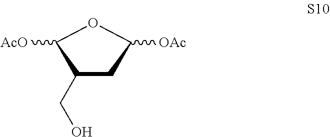

Compound S2 (163 mg, 0.886 mmol, 1 eq) was dissolved in EtOAc (16 mL, 50 mM). Rhodium on alumina catalyst (85.2 mg) was added to the pressure bottle equipped with a regulator. The vial was pressurized with H₂ to 70 psi, purged three times, and stirred at 25° C. for 2-4 h. After venting the pressure bottle, TLC (4:6 Hex/EtOAc, R_{f}=0.3, stained with PAA) showed the starting material was no longer present. When complete, the reaction mixture was passed through celite to remove the Rh catalyst. The filtrate was concentrated under vacuum to a pale, yellow residue, which was purified by column chromatography (1:1 DCM/EtOAc) resulting in 67 mg (410%) of a mixture of diastereomers (S10). ¹H NMR (400 MHz, CDCl₃) δ 6.44 (s, 1H), 6.34 (d, J=1.0 Hz, 1H), 3.86-3.75 (m, 1H), 3.69 (dt, J=5.5, 10.9 Hz, 1H), 2.56-2.43 (m, 2H), 2.05 (s, 3H), 2.04 (s, 3H), 1.90 (t, J=5.5 Hz, 1H), 1.82 (dd, J=1.0, 5.5 Hz, 1H).

### Preparation of S11 (see Paul et al., 2017)

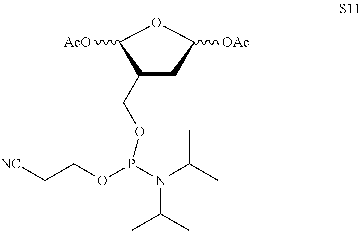

Compound S10 (67 mg, 0.28 mmol) was azeotropically dried with pyridine (2×0.5 mL). The flask was cooled to 0° C. DIPEA (0.2 mL, 144 mg, 1.12 mmol, 4 eq) was added to the cold starting material and the reactants were dissolved in DCM (1.2 mL, 200 mM). 2-Cyanoethyl-N, N-diisopropylchlorophosphoramidite (75 μL, 80 mg, 0.34 mmol. 1.2 eq) was added and the cold mixture stirred with periodic monitoring by TLC (7:1 Hex/EtOAc, R_{f}=0.2, stained with PAA). After 2 h, TLC showed complete conversion to the phosphoramidite. The reaction was diluted with EtOAc (10 mL). The organic layer was washed with saturated bicarbonate solution (2×15 mL) and the aqueous layers were extracted with EtOAc (2×20 mL). The combined organic layers were washed with brine (1×25 mL) and dried over Na₂SO₄, The organic layer was concentrated under vacuum and purified by column chromatography (3:1 hexanes/EtOAc) yielding 85 mg (75%) of S11. ¹H NMR (400 MHz, CDCl₃) δ 6.31 (m, 1H), 4.28 (m, 1H), 3.78 (m, 3H), 3.55 (m, 3H), 2.79 (m, 1H), 2.60 (m, 2H), 2.44 (m, 1H), 2.04 (m, 6H), 1.81 (m, 1H), 1.14 (m, 12H). ³¹P NMR (400 MHz, CDCl₃) δ 149.7, 149.5, 148.3, 148.1, 13.2.

### Preparation of Pro-14

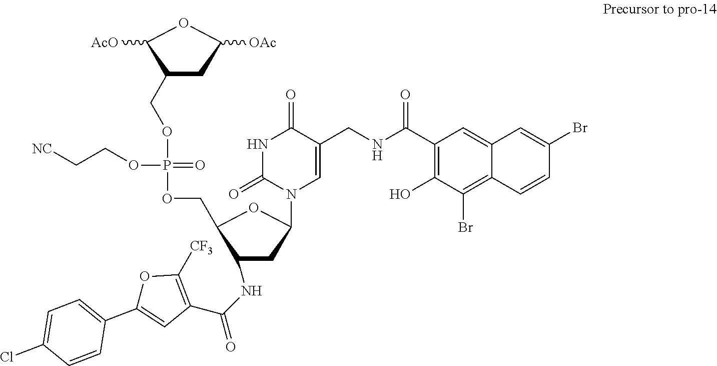

Compounds S9 (30 mg, 0.035 mmol) and S11 (35 mg, 0.042 mmol, 1.2 eq) were azeotropically dried together in pyridine (2×1 mL). S-ethyl tetrazole/MeCN (250 mM, 0.25 mL, 0.053 mmol, 1.5 eq) was added to the reaction flask. After 4 h, TLC (1:1 EtOAc/Hex) suggested the majority of S9 was consumed. tBuOOH (0.5 M, 10 mg, 25 μL, 0.11 mmol, 3 eq) was added for 20 min. The reaction was concentrated and purified by column chromatography (1:1 Hex/EtOAc->1:1 Hex/EtOAc, 5% MeOH) to yield 25 mg (60%) of the phosphate triester precursor to pro-14. ¹H NMR (400 MHz, CDCl₃) δ 8.16 (m, 1H), 7.87 (s, 1H), 7.78 (d, J=8.8 Hz, 1H), 7.50 (s, 1H), 7.29 (d, J=8.8 Hz, 1H), 6.95 (s, 1H), 6.75 (m, 1H), 6.36 (quint, J=3.2 Hz, 1H). 6.20 (s, 1H), 6.15 (m, 1H), 4.74 (q, J=5.6 Hz, 1H), 4.31 (m, 4H), 4.23 (m, 6H), 3.82 (d, J=5.6 Hz, 1H), 3.51 (m, 1H), 2.77 (m, 4H), 2.53 (m, 1H), 2.08 (s, 3H), 2.03 (s, 3H), 2.00 (m, 1H), 1.87 (m, 1H). ¹³C NMR (400 MHz, CDCl₃) δ 169.8, 100.8, 98.2, 60.2, 50.5, 32.2, 25.2, 20.9, 20.83, 20.77, 19.8, 19.7, 14.6, 14.0. ³¹P NMR (400 MHz, CDCl₃) δ 17.3, 14.3, 9.2, 9.1, 8.1, 7.9, 7.8, 7.75, 7.70, 7.6, 7.5, −0.5, −0.9, −1.4, −1.9, −2.0, −2.5, −2.6, −2.7, −2.8. ESI-TOF m/z calculated for C₄₅H₄₀Br₂ClF₃N₅O₁₆P (M+H)—1188.0215, 1188.0200 observed.

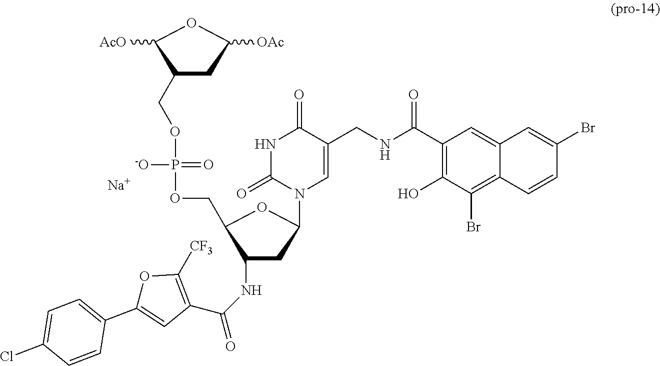

The intermediate phosphate triester (25 mg, 0.023 mmol) was dissolved in DMF (0.5 mL) and DIPEA (0.5 mL). The reaction was stirred at 25° C. overnight. The following morning, TLC (5% MeOH in DCM, R_{f}=0.2, UV active) indicated product formation. The reaction was concentrated and purified by column chromatography (2% MeOH in DCM->5% MeOH in DCM) and then passed through a TEA+ Dowex and then Na+ Dowex column to yield 12.4 mg (50%) of pro-14. A portion (˜30%) of the product exists at the triethylammonium salt, even after the Na+ ion exchange column. ¹H NMR (800 MHz, CD₃OD) a 8.53 (s, 1H), 8.12 (s, 1H), 8.02 (s, 1H), 7.92 (d, J=8.8 Hz, 1H), 7.78 (d, J=8.5 Hz, 2H), 7.56 (d, J=8.8 Hz, 1H), 7.53 (d, J=8.5 Hz, 2H), 7.24 (s, 1H), 6.30 (quint, J=5.4 Hz, 1H), 6.25 (m, 1H), 4.73 (t, J=6 Hz, 1H), 4.61 (q, J=6 Hz, 1H), 4.49 (d, J=14 Hz, 1H), 4.42 (dd, J=3.6, 14 Hz, 1H), 4.24 (m, 3H), 4.14 (m, 1H), 4.05 (t, J=7 Hz, 1H), 3.06 (q, J=8 Hz, 1H), 2.62 (m, 2H), 2.48 (q, J=6 Hz, 2H), 2.05 (m, 3H), 1.99 (s, 3H), 1.92 (s, 1H), 1.84 (m, 1H), 1.32 (t, J=8 Hz, 3H). ¹³C NMR (800 MHz, CDCl₃) δ 175.7, 170.4, 170.1, 168.6, 163.8, 161.7, 157.5, 154.3, 150.7, 139.5, 138.6, 135.0, 133.9, 131.3, 130.8, 129.0, 128.4, 127.1, 126.5, 125.7, 125.3, 119.6, 118.3, 115.0, 111.1, 106.2, 101.1, 100.0, 98.6, 96.7, 85.7, 83.3, 65.0, 63.4, 59.7, 50.2, 43.2, 37.1, 35.4, 32.0, 26.7, 24.7, 22.6, 19.7, 19.6, 19.5, 14.2. ³¹P NMR (400 MHz, CD₃OD) δ 6.4, 4.4, 4.3, 4.0, 3.9, 1.6, 1.5, 1.2, −0.26, −0.29, −0.4, −0.5. ESI-TOF m/z calculated for C₄₂H₃₇Br₂ClF₃N₄O₁₆P (M−H)—1132.9950, 1133.0007 observed.

### 1.7 Radiolabeling and Preparation of Oligonucleotide Complexes

Oligonucleotides were 3′-32P labeled by a32-P Cordycepin triphosphate and terminal deoxynucleotidyl transferase. 3′-32P labeling was also completed using Klenow exo- and a-32PATP, within a duplex containing a single 5′-dT overhang. In this case, the 3′-terminus of the 5′-dRP strand was 32P-labeled, denatured from the duplex, and purified by 20% denaturing PAGE. Labeling with 5′-32P labeling was completed by T4 polynucleotide kinase and g-32P-ATP. Ternary complexes were hybridized by mixing ³²P-labeled oligonucleotides with the appropriate template and flanking strand in a 1:2.5:5 ratio in phosphate buffered saline (10 mM sodium phosphate, 100 mM NaCl, pH 7.3), heating to 95° C., and slowly cooling to 25° C. Ternary complexes containing fluorophore labeled oligonucleotides were prepared by annealing the fluorophore-labeled strand with the appropriate quencher-labeled template and flanking strand in a 1:2:3 ratio. All oligonucleotides used to prepare ternary complexes are described in Table S1. Oligonucleotides containing a photochemical precursor (TC1) were generated fresh by photolysis (350 nm, 20 min) using a Rayonet. Guan and Greenberg, 2010.

TABLE S1
DNA substrates for assays.

| | |
|---|---|
| Pol β¹ (TC1) | 5′-d(TAA TGG CTA ACG CAA XAC GTA ATG CAG TCT)-3′ SEQ ID NO: 1 |
| | 3′-d(ATT ACC GAT TGC GTT_ATG CAT TAG GTC AGA)-S′ SEQ ID NO: 2 |
| Pol β² and | 5′-d(TCA CCC TCG TAC GAC TC TTT TTT TTT TGC F)-3′ SEQ ID NO: 3 |
| Klenow (TC2) | 3′-d(AGT GGG AGC ATG CTG AG_AAA AAA AAA AGG Q)-5′ SEQ ID NO: 4 |
| Pol θ (P3) | 5′-d(TTT TTT TAG GTT T)-3′ SEQ ID NO: 5 |
| Pol η (D4) | 5′-d(TCA CCC TCG TAG GAC TC)-3′ SEQ ID NO: 6 |
| | 3′-d(AGT GGG AGC ATG GTC AGG ACC T)-3′ SEQ ID NO: 7 |
| Pol λ (TC5) | SEQ ID NO: 8 |
| SEQ ID NO: 9 | 5′-d(ACC ATG GGA GGT GCT G ACT CCA CTA GAT AGA CTT)-3′ |
| | 3′-d(TGG TAC CCT GCA CGA CTC TGA GGT GAT GTA TGT GGA)-5′ |
| Fluorescence | 5′-d(TAA TGG GTA ACG CTT pFCC GTA ATG CAG TCT)-3′ SEQ ID NO: 10 |
| Anisotropy | 3′-d(ATT ACC GAT TGC GAA_AGG CAT TAC GTC AGA Fl)-5′ |
| (TC6) | SEO ID NO: 11 |

X = dRP,
F = TAMRA,
Q = BHQ,
Fl = dichloro-diphenyl-fluorescein (SIMA-HEX)
pF =
¹= lyase assay,
²= fluorescence assay

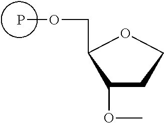

### 1.8 General Procedure for Library Screening

A 1 mM stock solution of each inhibitor is prepared using 50% MeCN in H₂O. A solution of Pol β (100 nM) was preincubated with library compounds (50 μM) in 1× reaction buffer (total volume: 50 μL; 50 mM HEPES buffer pH=7.4, 5 mM MgCl₂, 0.2 mM EDTA, 50 mM KCl, 0.01% Tween 20, 0.01 mg/mL BSA, and 4% glycerol by volume) in a 384-well plate at 25° C. for 30 min. In control experiments, an equal volume of a control solution (containing all coupling and deprotection reagents but lacking inhibitor) was added to keep the percentage of solvents and reagents consistent. An aliquot (3 μL) was diluted with a 2× solution (15 μL) containing TC2 (100 nM, Table S1) and dTTP (200 μM) in 1× reaction buffer (total volume: 30 μL) in a different 384-well plate. The final reaction mixture contained 10 nM Pol β, 5 μM inhibitor, 50 nM DNA, 100 μM dTTP, 1× reaction buffer, and 0.25% MeCN. The solution in each well was mixed thoroughly, and the fluorescence measurements were collected immediately.

### 1.9 Time-Dependent Irreversible Kinetics of Pol β

A 1 mM stock solution of each inhibitor is prepared using 50% MeCN in H₂O. A solution of Pol β (50 nM) was preincubated with 14 (0, 100, 250, 400, 500, 750 nM) in 1× reaction buffer (total volume: 50 μL; 50 mM HEPES buffer pH=7.4, 5 mM MgCl₂, 0.2 mM EDTA, 50 mM KCl, 0.01% Tween 20, 0.01 mg/mL BSA, and 4% glycerol by volume) in a 384-well plate at 25° C. for various preincubation times (2, 5, 10, 15, 20 min). In control experiments, an equal volume of a control solution (containing all coupling and deprotection reagents but lacking inhibitor) was added to keep the percentage of solvents and reagents consistent. The volume of inhibitor solution added was unchanged across experiments that used various inhibitor concentrations. To achieve various inhibitor concentrations, the stock solution of inhibitor (1 mM) was diluted appropriately for the desired conditions. An aliquot (3 μL) was diluted with a 2× solution (15 μL) containing TC2 (100 nM, Table S1) and dTTP (200 μM) in 1× reaction buffer (total volume: 30 μL) in a different 384-well plate. The final reaction mixture contained 5 nM Pol β, 14 (0, 10, 25, 40, 50, 75 nM), 50 nM DNA, 100 μM dTTP, 1× reaction buffer, and 0.25% MeCN. The solution in each well was mixed thoroughly, and the fluorescence measurements were collected immediately. The data were fit to a single exponential growth equation (1) that follows a plateau. The plateau was important because the strand displacement assay exhibited an induction period in which several nucleotides of the fluorescently labelled DNA were displaced before the fluorescently labeled oligonucleotide was released into solution. This induction time was determined by inspection and typically varied between 10 and 15 min. The data were fit beginning at the time when a growth in fluorescence was observed. Zhang and Seelig, 2011; Olson et al., 2017.

$$Y = {F}_{0} + ( {F}_{1} - {F}_{0} ) \times ( 1 - {e}^{- kt} ) \qquad ( 1 )$$

Y is the fluorescence intensity, F₀ is the fluorescence value at time 0, F₁ is the fluorescence value at time ∞, k is the rate constant, and t is time. Rate constants are extracted for each experiment and relative rates are determined using equation (2).

$${k}_{r e l} = {k}_{inhibitor} / {k}_{pol β} \qquad ( 2 )$$

Where k_{inhibitor} is the rate constant for experiments containing inhibitor and k_{polβ} is the rate constant for control experiments lacking inhibitor. This procedure was also used to measure strand displacement activity of Klenow exo− with minor changes: (1) the concentration of 14 during preincubation was 0.5 or 10 μM, and (2) the samples were preincubated for 20 min.

### 1.10 Gel-Based Lyase Assay

A solution of Pol β (20 nM) was preincubated with inhibitor at various concentrations (e.g., 5 μM) in 1× reaction buffer (total volume: 50 μL; 50 mM HEPES buffer pH=7.4, 5 mM MgCl₂, 0.2 mM EDTA, 50 mM KCl, 0.01% Tween 20, 0.01 mg/mL BSA, and 4% glycerol by volume) at 25° C. for 30 min. In control experiments, an equal volume of a control solution (containing all coupling and deprotection reagents but lacking inhibitor) was added to keep the percentage of solvents and reagents consistent. The volume of inhibitor added was unchanged across experiments that used various inhibitor concentrations. To achieve various inhibitor concentrations, the stock solution of inhibitor (1 mM in 50% MeCN in H₂O) was diluted appropriately for the desired conditions.

An aliquot (3 μL) was mixed with of freshly prepared TC1 (150 nM, Table S1) in 1× reaction buffer (total volume: 30 μL). The final mixture in the reaction well contained 2 nM Pol β, 0.5 μM inhibitor, 150 nM DNA, and 1× reaction buffer. Aliquots (4 μL) were removed at various times (0, 2, 5, 10, 15, 20 min) and flash frozen in dry ice. Afterwards, the thawed mixtures were immediately stabilized by NaBH₄ (4 μL, 300 mM) for 2 h at 4° C. The samples were mixed with formamide loading buffer (8 μL, 90%, 10 mM EDTA). An aliquot (6 μL) was loaded onto a 20% denaturing polyacrylamide gel. The gel was exposed in a radiography cassette and the product was analyzed using a Phosphorimager.

This procedure was also used to measure lyase activity of 8 kDa Pol β with minor changes: (1) The concentration of 8 kDa Pol β during preincubation was 100 nM (2) After preincubation, an aliquot (3 μL) was mixed with of freshly prepared TC1 (20 nM, Table S1) in 1× reaction buffer (total volume: 30 μL). Therefore, the reaction mixture contained 10 nM 8 kDa Pol β and 20 nM TC1, along with the other components.

### 1.11 Primer Extension Assay (Pol θ) (see He and Yang, 2018)

A solution of 10×14 (5 μM or 100 μM, 2 μL) was mixed with a 40× solution of Pol θ (200 nM, 5 μL) in 1× reaction buffer (20 mM Tris·HCl pH 7.5, 100 mM NaCl, 5 mM MnCl₂, 0.5 mM TCEP, 10% glycerol, 0.01% NP-40, 0.1 mg/mL BSA) in a 384 microtiter well plate. This 10× preincubation mixture (50 nM Pol q, 0.5 or 10 μM 14) was incubated at 25° C. for 20 min. An aliquot of the 10× preincubation mixture (2 NL) containing Pol θ and 14 was added to a new well and mixed with 1× buffer (8 μL) and 2× cocktail solution (10 L) containing DNA substrate P3 (1 μM. Table S1), dNTPs (0.8 mM each), and 2×SYBR Gold in 1× buffer (Table 1). The final reaction mixture (20 μL) contained 5 nM Pol θ, 0.05 or 1 μM 14, 500 nM P3, 0.4 mM dNTPs, 1× Sybr Gold in 1× reaction buffer. Fluorescence data was collected for 80-120 min on a Varian Cary Eclipse fluorescence spectrophotometer.

### 1.12 Primer Extension Assays (Pol η and Pol λ) (see Gowda et al., 2017)

A 10× working solution of 14 (5 or 100 μM) was prepared in 1:1 MeCN/H₂O. A 10× preincubation mixture was prepared by mixing a 50× working solution of polymerase (250 nM) with an aliquot of the inhibitor (5 or 100 μM, 10×) in 1× reaction buffer (50 mM Tris·HCl, 50 mM NaCl, 5 mM MgCl₂, 5 mM DTT, 0.1 mg/mL BSA, 10% glycerol, pH 7.5) (see Table 19 for volumes). The concentration of the preincubation mixture was 50 nM polymerase (10×) and 0, 0.5 or 10 μM 14 (1×).

TABLE S2
10X preincubation mixture for primer extension assays.

| | Pol η | Pol λ |
|---|---|---|
| | (volume added, μL) | (volume added, μL) |
| 50X Polymerase (250 nM) | 2 | 3 |
| 10X 14 (5 or 100 μM) (or | 1 | 1.5 |
| 1:1 MeCN/H₂O for control) | | |
| 20X reaction buffer | 1 | 1.5 |
| H₂O | 6 | 9 |
| Total¹ | 10 | 15 |

¹Preincubated at 25° C. for 20 min.

The samples containing 50 nM polymerase and 14 (0, 0.5, or 10 μM) were preincubated at 25° C. for 20 min. After preincubation, an aliquot (2 μL, pol η or 3 μL, pol λ) was diluted with 10× ternary complex (pol η: 2 μL, 500 nM D4 or pol λ: 3 μL, 100 nM TC5), 10× dNTPs (5 mM; 2 μL, pol η or 3 μL, pol λ), and 1× reaction buffer (14 μL., pol η or 21 μL, pol λ) (see Table 20 for volumes). While the samples were incubated at 37° C., aliquots (pol η: 0, 2, 5, 10, 15, 20 min, 3 μL or pol λ: 0, 5, 15, 20, 30 min, 5 μL) were removed and quenched by the addition of 95% formamide, 20 mM EDTA loading buffer (10 μL). Aliquots were heated at 95° C. for 5 min, spun down, and loaded onto a 20% denaturing PAGE and run for 4 h at 55 watts. The gel was exposed in a radiography cassette, which was scanned using a Phosphorimager.

TABLE S3
Reaction mixture for primer extension assays.

| | Pol η | Pol λ |
|---|---|---|
| | (volume added, μL) | (volume added, μL) |
| 10X preincubation | 2 | 3 |
| 10X DNA | 2 (500 nM D4) | 3 (100 nM TC5) |
| 10X dNTPs (5 mM) | 2 | 3 |
| 1X reaction buffer | 14 | 21 |
| Total¹ | 20 | 30 |

¹Incubated at 37° C., taking time points between 0-30 min.

### 1.13 General Procedure for Dialysis

Pol β (100 nM, total volume 200 μL) was preincubated in the absence or presence of 14 (e.g., 750 nM) in 1× reaction buffer (50 mM HEPES buffer pH=7.5, 5 mM MgCl₂, 0.2 mM EDTA, 50 mM KCl, 0.01% Tween 20, 0.01 mg/mL BSA, and 4% glycerol) at 25° C. for 20 min. The strand displacement activity of each sample was immediately measured by mixing an aliquot of each sample (3 μL, 100 nM Pol β, ±750 nM 14) with a 2× solution (15 μL) containing TC2 (100 nM) and dTTP (200 IM) in 1× reaction buffer (total volume: 30 μL). The final concentrations during kinetics were 10 nM Pol β, 750 nM 14, 50 nM TC2, and 100 μM dTTP. The remaining sample (197 μL) was dialyzed in a 3.50K MW cassette in reaction buffer (1 L, buffer exchanged after 12 h) containing 50 mM HEPES buffer (pH=7.4, 5 mM MgCl₂, 4 mM DTT) for 24 h. The volume of the solution in the cassette was marked and no considerable volume change was observed after dialysis. The remaining strand displacement activity of the enzyme was measured as previously described. An aliquots (3 μL) was mixed with a 2× solution (15 μL) containing TC2 (100 nM) and dTTP (200 NM) in 1× reaction buffer (total volume: 30 μL). This method was also used to analyze the effect of pH on the inhibitory activity of 14 with one minor change: the dialysis buffer contained 50 mM HEPES buffer (pH=8.0, 5 mM MgCl₂, 4 mM DTT).

### 1.14 UPLC MS/MS Analysis of Pol β Modification by Covalent Inhibitors

A solution of Pol β (25 μL, 20 μM, 500 μmol) was mixed with 14 (5 μL, 30 μM, 100×), H₂O (420 μL) and 10× reaction buffer (50 μL, 500 mM HEPES buffer, pH=7.4, 50 mM MgCl₂, 20 mM DTT) and incubated at 25° C. for 30 min. The reaction mixture was concentrated by centrifugation using an Amicon 3K centrifugal filter. To prevent the loss of protein, the centrifugal was blocked with Pol β prior to addition of the sample. Blocking was conducted by adding Pol β (500 μL, 0.5 μM), followed by centrifugation (13,000 g, 25 min, 4° C.) and removal of the supernatant. Following blocking of the membrane filter, the sample (500 μL, 1 μM Pol β±300 nM 14) was added to the Amicon centrifugal filter, and centrifugation was carried out (13,000 g, 25 min, 4° C.). The sample was then washed twice with 500 μL of 1× reaction buffer can concentrated by centrifugation in the Amicon filter to 50 μL (10 μM Pol θ). Digestion buffer (25 μL, 500 mM Tris-HCl pH 7.4), 10× trypsin (25 NL, 400 μM), and H₂O (150 μL) were added to yield a final mixture of 2 μM Pol β and 40 RM trypsin (1:20 ratio) in 1× digestion buffer (total volume, 250 μL, 50 mM Tris-HCl, pH 7.4). The digestion sample was incubated at 37° C. overnight. A portion (100 μL) of the digestion mixture was spun down (16,000 g, 10 min, 4° C.). The sample (10 μL) was injected onto and analyzed by UPLC-MS/MS using an ACQUITY UPLC HSS T3 Column (100 Å, 1.8 m, 2.1 mm×100 mm). The flow rate was 0.3 mL/min running a gradient from 85:5:10 water:acetonitrile: 1% formic acid to 50:40:10 water:acetonitrile: 1% formic acid over 35 min. Analysis was conducted using BioPharmaLynx with tolerance set to 30 ppm and allowing for 4 missed cleavages.

### 1.15 Fluorescence Anisotropy (see Laverty et al., 2018)

Anisotropy measurements were conducted using a solution of dichloro-diphenylfluorescein-labeled TC6 (2.5 nM, Table S1) and Pol β (varying concentrations) in reaction buffer (50 mM HEPES, pH 7.5, 20 mM KCl, 1 mM EDTA, and 1 mM (3-mercaptoethanol). Samples also contained 10% storage buffer (20 mM Tris·HCl, pH 7, 300 mM NaCl, 10% glycerol, 5 mM BME) by volume. In a typical experiment, a sample (300 NL) was prepared by mixing Pol β (30 μL, 1 μM) in storage buffer with 10× reaction buffer (30 μL), TC6 (30 μL), a solution 50% MeCN in H₂O containing or lacking 14 (200 PM, 3 L) and H₂O (207 μL). These samples, termed solutions A and A′ (A did not contain 14 and A′ contained 2 μM 14), contained 250 μM TC6, 100 nM Pol β, ±2 μM 14. Samples containing various concentrations of Pol β were prepared by serial dilution with solution B and B′. Solution B (10 mL) was prepared by mixing H₂O (7.85 mL), with 10× reaction buffer (1 mL), 10× storage buffer (1 mL), TC6 (50 nM, 50 μL), and a solution of 50% MeCN in H₂O containing or lacking 14 (200 μM, 100 μL). Similarly, solution B did not contain 14 and was used exclusively to dilute solution A, whereas solution B′ contained 2 μM 14 and was used to dilute solution A′. By mixing equal volumes of A or A′ (150 μL) with B or B′ (150 μL) respectively, the concentration of Pol β decreased to 50 nM, while the concentration of DNA and 14 remain unchanged. An aliquot (150 μL) of this new solution was then mixed with solution B or B′ (150 μL) to prepare anew solution containing 25 nM Pol β. Serial dilutions were repeated such that samples contained Pol β concentrations of 100 nM, 50 nM, 25 nM, 12.5 nM, 6.25 nM, 3.13 nM, 1.56 nM, 0.78 nM, 0.39 nM, and 0.2 nM. Samples were incubated at 25° C. for 1 h and fluorescence anisotropy (A) was measured using a portion (125 μL) of each sample with a PMT voltage of 800 mV, 8 nm slit width, 535 nm excitation and 556 nm emission. Fluorescence anisotropy was measured for TC6 in the absence of enzyme (A0), and the change in anisotropy (A-A0) was calculated for each sample and plotted against the concentration of Pol β. Each fluorescence anisotropy measurement was collected in triplicate.

### 1.16 Growing Conditions for Different Cell Lines

Mouse embryonic fibroblast cells were grown in DMEM with high glucose supplemented with 9% FBS at 34° C. in a 10% CO₂ humidified incubator. HeLa cells were grown in DMEM with high glucose supplemented with 9% FBS and 1% antibiotic antimycotic solution (penicillin, streptomycin, and amphotericin B). HeLa cells were grown at 37° C. 5% CO₂ in a humidified incubator.

### 1.17 Clonogenic Assay for Cell Survival

Approximately 2×10⁵ HeLa cells were plated in each well of a 6-well culture plate (well size; 35 mm×18 mm) in DMEM containing 10% FBS (1 mL) and kept in a humidified incubator at 37° C. with 5% CO₂. After overnight incubation, cells were subjected to either the vector (50% MeCN in H₂O) or treatment (100× pro-14, 10 NL; in 50% MeCN in H₂O and/or 100×DNA damaging agent (e.g., 20 mM MMS, 200 μM BLM; 10 μL; in DMEM-FBS medium). For alkylation experiments, cells were incubated with MMS (0 or 0.2 mM), with or without pro-14 (5, 25 μM) at 37° C. with 5% CO₂ for 1 or 2 h. After treatment, the medium was removed, and the cells were washed with 1×PBS (2×1 mL). The cells were trypsinized with 0.25 w/v Trypsin EDTA (1 mL in each well, 5 min incubation at 37° C.), washed with DMEM-FBS (10 mL) to quench the trypsin cleavage, and spun down (3,000 RCF×5 min). The medium was removed, and the cells were resuspended with fresh DMEM-FBS (10 mL). The single cell suspensions were collected and counted using a TC20 Automated Cell Counter (BIO-RAD). Stock solutions of single cell suspensions were prepared for all untreated and treated cells. For example, 100 cells/mL stock solution of untreated cells were prepared; 500 cells/mL stock solutions of treated cells were prepared. The concentration of stock solution for each sample was determined based on expected toxicity of the treatment (i.e., higher concentrations for more toxic conditions). The appropriate number of cells for each experiment were seeded in each well of a 6-well plate (well size; 35 mm×18 mm) in 3 mL of DMEM-FBS medium. The cells were grown in a humidified incubator at 37° C. with 5% CO₂ for 14 days. No significant change in media volume was observed after two weeks due to evaporation. After 14 days, the growth medium was discarded, and the attached cells were treated with 0.2% w/v crystal violet solution. The excess dye was washed with water. The plates were dried and scanned with an HP Scanjet 3970 and colonies were counted using ImageJ (FIJI). Plating efficiencies (PE) and survival fractions (SF) were calculated as follows: PE=number of colonies/number of cells seeded; SF=PE/PE_{control}.

### 1.18 Cell Viability Assays for MEFs (see Breathwaite et al., 2010; Hu et al., 2004)

Mouse embryonic fibroblasts (Pol β1 WT, Pol β−/−, Pol λ WT, Pol λ−/−, Pol β−/λ−) were seeded at a density of ˜0.3×10⁶ cells/well in 6-well dishes. The following day, cells were exposed for 1 h to a range of MMS concentrations (0, 0.1, 0.2, 0.5, 1.0, 1.5 mM) in growth medium in the presence of absence of pro-14 (0, 5, 15, 25 μM). Control wells were treated with an equal volume of vector (50% MeCN in H₂O). Cells were washed with 1×PBS and fresh medium was added. Dishes were incubated for 5 days at 34° C. in a 10% CO₂ incubator until untreated control cells were approximately 80% confluent. Cells (triplicate wells for each treatment concentration were counted by a cell lysis procedure (described previously), and the results were expressed as the surviving fraction of cells in drug-treated wells relative to control wells.

### Example 2

### Suppression of DNA Polymerase β Activity is Synthetic Lethal in BRCA1 Deficient Cells

### 2.1 Overview

People whose cells express mutated forms of the BRCA1 tumor suppressor are at higher risk for developing cancer. BRCA1 deficient cells are defective in DNA double-strand break repair. Inhibiting poly(ADP-ribose) polymerase 1 (PARP1) in such cells is synthetic lethal, a cytotoxic effect, that has been exploited to produce anticancer drugs, such as Olaparib. Alternative synthetic lethal approaches, however, are necessary. The presently disclosed subject matter, in some embodiments, discloses that DNA polymerase β (Pol β) forms a synthetic lethal interaction with BRCA1. SiRNA knockdown of Pol β in BRCA1 deficient ovarian cancer cells, or treatment with a Pol β pro-inhibitor (pro-14) is cytotoxic. BRCA1 complemented cells are significantly less susceptible to either treatment. Pro-14 also is toxic to BRCA1 deficient breast cancer cells and its toxicity in BRCA1 deficient cells is comparable to that of Olaparib. These experiments establish Pol β as a synthetic lethal target within BRCA1 deficient cells and a potentially useful one for treating cancer.

### 2.2 Background

Cells that are deficient in DNA double-strand break repair face a significant increase in risk of becoming cancerous. The breast cancer type 1 and 2 (BRCA1/2) gene products are examples of tumor suppressors involved in homologous recombination (HR), a double-strand break repair pathway, which when mutated give rise to significant increases in cancer incidence. Roy et al., 2012. Although mutations in BRCA1 increase susceptibility to breast, ovarian and to lesser extents melanoma, prostate and pancreatic cancers, they also provide a target of opportunity for selective treatment. Inhibiting poly(ADP-ribose) polymerase 1 (PARP1), an enzyme involved in DNA repair, is significantly more lethal in BRCA1 deficient cells than in healthy ones.

This example of synthetic lethality has given rise to anew generation of anti-cancer agents, including Olaparib, Niraparib and Rucaparib, which are selectively cytotoxic to cancer cells that are HR deficient (HRD). O'Neil et al., 2017; Huang et al., 2020. Unfortunately, not all cancers respond to these treatments and others develop resistance. Hence, there is a need for additional synthetic lethal targets to selectively target HRD cells. Higgins and Boulton, 2018. DNA polymerase θ is one such target, as is the nucleosome remodeling protein ALC1. Ceccaldi et al., 2015; Hewitt et al., 2021. Without wishing to be bound to any one particular theory it is thought that inhibiting DNA polymerase β (Pol β) would be synthetic lethal in BRCA1 deficient cells. Nickoloff et al., 2017.

### 2.2 Result and Discussion

The presently disclosed subject matter, in some embodiments, demonstrates that inhibiting Pol β. or suppressing its expression using siRNA, is a viable synthetic lethality approach to selectively kill BRCA1 deficient cells. Pol β is a bifunctional polymerase that is most well-known for its roles in base excision repair (BER) within the nucleus (Scheme 8A). Beard and Wilson, 2006.

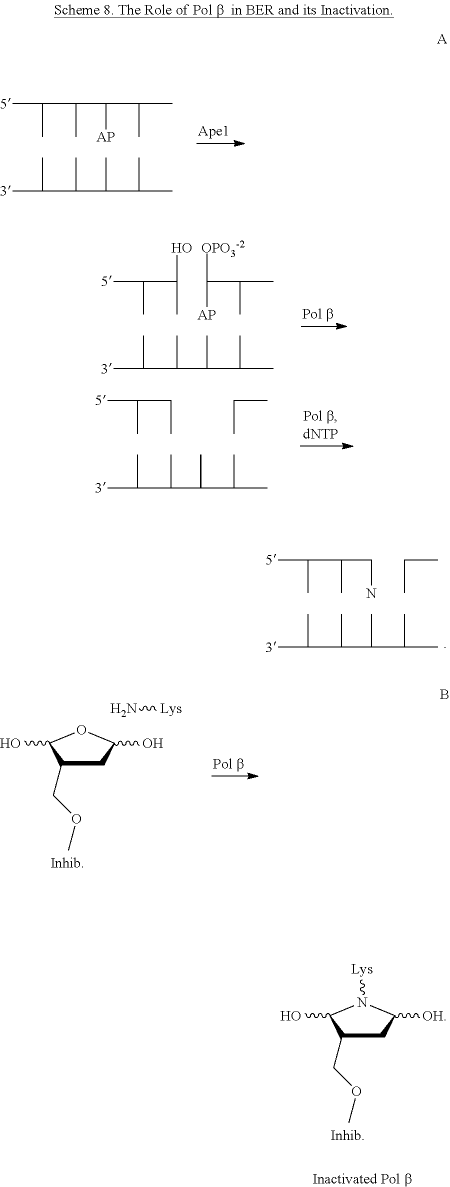

Pol β removes the remnants of an abasic site (AP) following incision by apurinic endonuclease 1 (Ape1), and in short patch BER extends the 3′-terminus of the cleaved DNA strand to fill in the resulting gap. More recently, Pol β has been shown to also be involved in double-strand break repair and mitochondrial DNA repair. Ray et al., 2018; Sykora et al., 2017; Prasad et al., 2017; Baptiste et al., 2021. Pol β is overexpressed in many human cancers, including colon cancer where approximately 40% of tumors contain mutated enzyme. Donigan et al., 2012. For these reasons, Pol β is an increasingly popular inhibition target. Nickoloff et al., 2017; Barakat et al., 2012; Gowda et al., 2017; Strittmatter et al., 2014; Jaiswal et al., 2015.

To this end, a platform for identifying mechanism-based covalent inhibitors of Pol β from chemical libraries has been developed. (Scheme 8B). Arian et al., 2014; Paul et al., 2017; Paul et al., 2018. This strategy was developed, in part, in view of potent cytotoxic antitumor agents that produce 1,4-dicarbonyl containing DNA lesions, which inactivate Pol β. Guan and Greenberg, 2010; Jabobs et al., 2011. Recently, this approach provided a covalent inhibitor (14) that inactivates Pol β by modifying a lysine in the polymerase active site, which then prevents DNA binding. Yuhas et al., submitted.

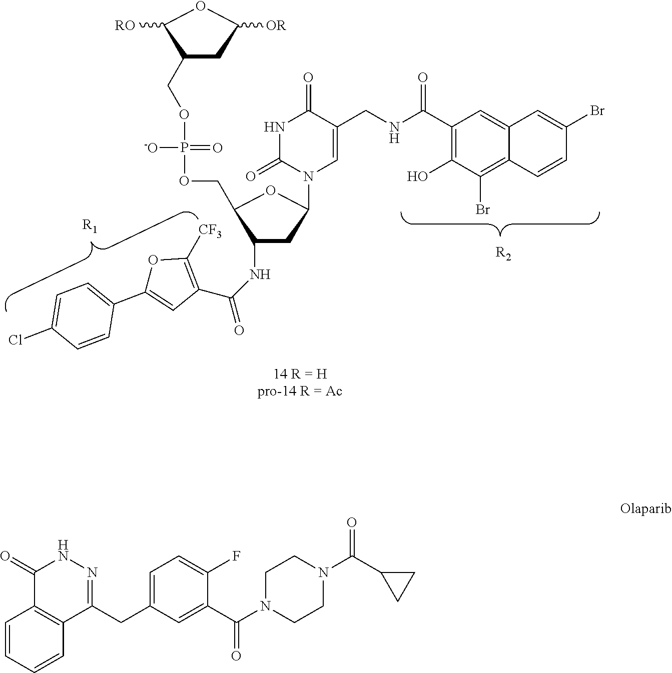

Inhibitor 14 is selective for Pol β over other polymerases, including the back-up repair enzyme Pol λ, and the corresponding pro-inhibitor (pro-14) is not toxic (<10% cell death) by itself but works synergistically with DNA damaging agents to kill HeLa cells by targeting Pol β, the presently disclosed subject matter used siRNA to knock down Pol β and pro-14 in separate experiments to demonstrate synthetic lethality with BRCA1.

Synthetic lethality is defined by a relationship between two proteins, such that while loss of function of either one is not cytotoxic, loss of function of both is. O'Neil et al., 2017; Huang et al., 2020. Consequently, a synthetic lethal partner for a nonfunctional, mutated protein in a cancer cell is an attractive pharmacological target. Pol β has been speculated to be a synthetic lethal partner for BRCA1. Nickoloff et al., 2017. It was reported that suppression of Pol β and BRCA2 activities is synthetic lethal. Ali et al., 2021.

One aspect of the presently disclosed subject matter is to determine whether deficiencies in Pol β and the tumor suppressor BRCA1 are synthetically lethal. Polβ deficiency was introduced in cells in one of two ways. Pol β was knocked down using siRNA, as previously reported. Ray et al., 2018. Alternatively, a bis-acetate pro-inhibitor form of 1 (pro-1) was used. Yuhas et al., submitted. Compound 1 is a recently reported covalent inhibitor of Pol β that reacts with lysine residues in the polymerase domain to prevent DNA binding. When administered to cells as pro-1, cytotoxicity results from selective interaction with the target Pol β.

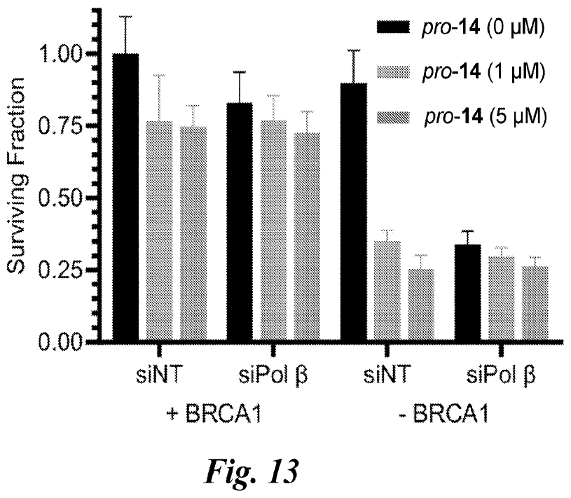

The isogenic ovarian cancer cell line (UWB1.289) that lacks BRCA1 (UWB1.289 (BRCA1−/−)) or is BRCA1 complemented (UWB1.289+BRCA1) provided an excellent environment in which to probe for a synthetic lethal interaction between Pol β and BRCA1 (FIG. 13). The effect of pro-1 (2 h treatment) on cell survival, as determined via clonogenic assay, was examined with and without siRNA knockdown of Pol β (siPol β). Western blotting indicated that Pol β was knocked down by approximately 80% following siPol β treatment (FIG. S1). Non-targeting siRNA (siNT) was used to validate siPol β effects. SiPol β had a modest cytotoxic effect on BRCA1 complemented ovarian cancer cells (UWB1.289+BRCA1). It is notable that the surviving fraction of BRCA1 complemented cells treated with siNT and pro-1 (1, 5 μM) were within error of those in which Pol β was knocked down with siPol β. Furthermore, the surviving fraction of siPol β treated cells did not decrease upon administration of pro-1. These observations are consistent with selective targeting of the covalent inhibitor for Pol β, as reported in mouse embryonic fibroblasts. Yuhas et al., submitted. This selectivity also was observed in experiments utilizing cells lacking BRCA1 (UWB1.289 (BRCA1−/−)).

Application of pro-1 (5 μM) to siNT treated BRCA1 deficient cells resulted in 75% cytotoxicity. In comparison, approximately 33% of the cells survived when approximately 80% of Pol β was knocked down by siPol β. Treatment of the siPol β knocked down cells with pro-1 (5 μM), however, resulted in the same level of toxicity observed in siNT treated BRCA1 deficient cells treated with the proinhibitor. This observation again, supports the hypothesis that pro-1 selectively targets Pol β in cells. Most importantly, the detrimental effects of pro-1 and siPol β on BRCA1 deficient cell survival are fully consistent with a synthetic lethal interaction between Pol β and BRCA1.

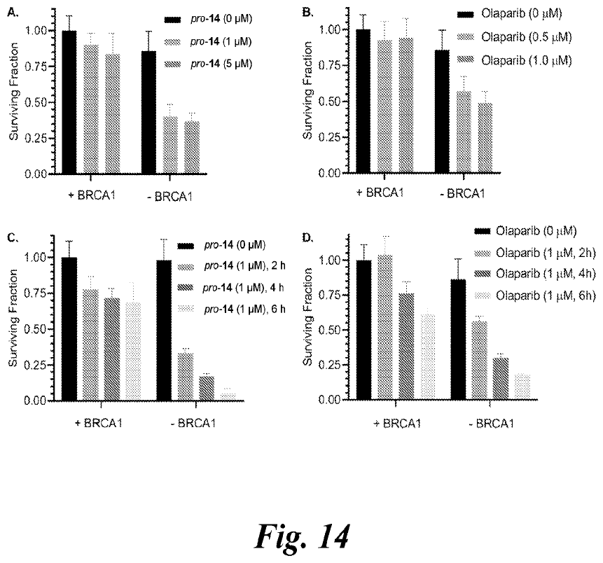

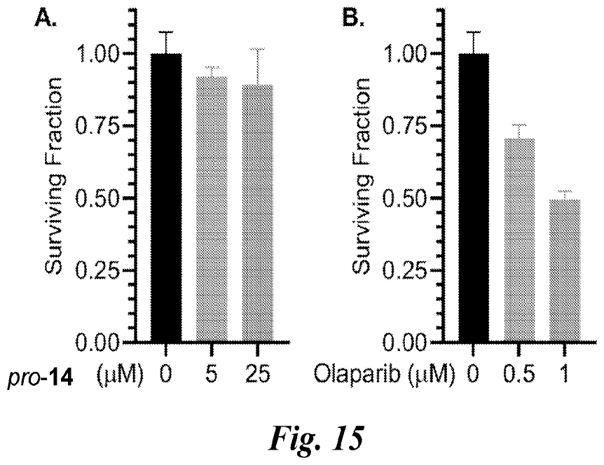

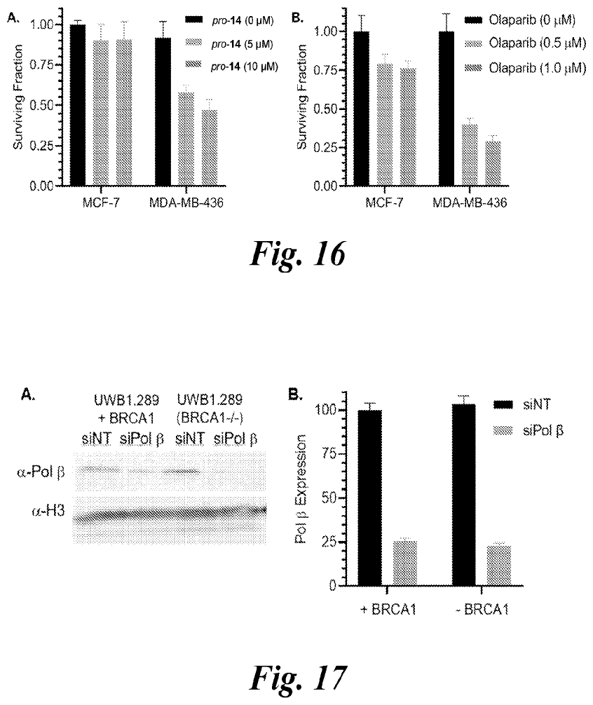

Having established the synthetic lethal interaction between Pol β and BRCA1, the cell killing effectiveness of this pair was compared with that involving PARP1 and BRCA1. Consequently, the cytotoxicity of pro-1 and Olaparib were compared in two cell lines. An initial comparison was made using the isogenic ovarian cancer cell line (UWB1.289). Again, pro-1 (1 h treatment) was only modestly toxic in BRCA1 complemented cells (FIG. 14A), as was Olaparib (FIG. 14B). Only 40% of the BRCA1−/− cells, however, survived treatment with either 1 or 5 μM of pro-1. The surviving fraction of the cells treated with pro-1 (1 μM) was slightly smaller than when treated with the same concentration of Olaparib. Cell death attributable to a similar covalent Pol β inhibitor was shown to increase with exposure time. Paul et al., 2017; Paul et al., 2018. This proved to be true regarding the cytotoxicity of pro-1 (1 μM) and Olaparib (1 μM) in BRCA1 deficient cells as well (FIG. 14C, FIG. 14D). While the cytotoxicity of both inhibitors was proportional to incubation time, the surviving fraction of BRCA1 deficient cells after 6 h exposure to pro-1 was more than 3-fold smaller than when Olaparib was employed. These data support synthetic lethality between homologous recombination and Pol β repair pathways. Furthermore, the data indicate that pro-1 is of comparable or even greater efficacy to FDA-approved Olaparib in this cell line. The time course analysis (FIG. 14C, FIG. 14D) also indicated that upon prolonged treatment, Olaparib was more cytotoxic to BRCA1 complemented ovarian cancer cells than pro-1, suggesting that the Pol β inhibitor is more selective in this cell line. As a point of comparison, the cytotoxicity of pro-1 and Olaparib were compared in a noncancerous cell line, mouse embryonic fibroblasts (MEFs) (FIG. 15). Exposing MEFs for 1 h to pro-1 at higher concentration than Olaparib resulted in less cytotoxicity, raising the possibility that the Pol β inhibitor may be less toxic in cells in which a synthetic lethal interaction is absent. The data supporting a synthetic lethal interaction between Pol β and BRCA1 were corroborated in breast cancer cell lines that are BRCA1 proficient (MCF-7) or deficient (MDA-MB-436) (FIG. 16). The toxicity (approximately 10%) of pro-1 in BRCA1+/+ cells (MCF-7) was within error of that observed in BRCA1 complemented UWB1.289 cells (FIG. 14A), albeit under slightly different conditions in which the proinhibitor was employed at as high as 10 μM (FIG. 16A) for the same amount of time (1 h). The survival fraction of BRCA1−/− breast cancer cells (MDA-MB-436), however, was less than 60% and 50% when treated with 5 and 10 μM pro-1, respectively. Olaparib was more effective at killing BRCA1−/− cells at a lower concentration (FIG. 16B). It also was more toxic to the BRCA1+/+MCF-7 cells, however. Pol β plays a vital role in base excision DNA repair pathway. Inhibiting the enzyme sensitizes cells to DNA damaging agents that produce nucleic acid modifications repaired by this pathway. Because Pol β is expressed constitutively in all cells, relying upon the synergistic cytotoxicity of a Pol β inhibitor and DNA damaging agent to kill cells may not provide high selectivity for cancer cells. Synthetic lethality provides a way to selectively kill cancer cells. Accordingly, the presently disclosed subject matter establishes Pol β and BRCA1 as a synthetic lethal pair, which further validates the attractiveness of this polymerase as an anticancer target.

### 2.3 Experimental

### 2.3.1 General Methods

Dulbecco's Modified Eagle Medium (DMEM) with high glucose, RPMI 1640 Medium with GlutaMAX supplement, Opti-MEM reduced serum medium, and insulin were obtained from ThermoFisher. Human Mammary Epithelial Cell Growth Medium (MEGM) was purchased from Sigma-Aldrich. Antibiotic antimycotic solution (penicillin, streptomycin, and amphotericin B), and fetal bovine serum (FBS) were obtained from MilliporeSigma. PBS buffer was obtained from Quality Biological. Trypsin-EDTA (0.25%) solution was purchased from ThermoFisher. Pre-cast 4-20% SDS-PAGE, Precision Plus Protein WestemC standard, Western blot kit, and StrepTactin-HRP conjugate were from BioRad. NP-40 was obtained from Sigma Aldrich. Lipofectamine RNAiMAX was purchased from ThermoFisher. siRNAs (siGENOME RISC-Free Control, D-001220-01; siGENOME Human POLB siRNA, D-005164-04) were purchased from Horizon Discoveries. Recombinant Anti-DNA Polymerase beta antibody (ab175197) was purchased from Abcam. Cells were counted using a BioRad TC20 cell counter. Western blots were carried out using a BioRad Trans-Blot Turbo Transfer system. Ponceau red stain was prepared with ponceau S tetrasodium salt (0.1%, Sigma Aldrich) and acetic acid (5%) in distilled H2O. Pierce ECL Western Blotting Substrate developing reagents were acquired from ThermoFisher. Western blots were visualized using a Typhoon 9410 equipped with chemiluminescence imaging at the Integrated Imaging Center at Johns Hopkins University.

UWB1.289 and UWB1.289+BRCA1 cells were provided by Prof. Peter Glazer, Yale University. Mouse embryonic fibroblasts (Pol β WT and Pol β−/−) were generous gifts from Dr. Sam Wilson, NIH. MDA-MB-436 cells were obtained from Prof. Theodore DeWeese, Johns Hopkins University.

Olaparib was purchased from Selleckchem. Solutions (100×) of pro-14 and Olaparib were prepared in 50% MeCN in H₂O.

### 2.3.2 Growth Conditions for Cell Lines

UWB1.289 (BRCA1−/−) and UWB1.289+BRCA1 cells were grown in a 1:1 mixture of RPMI-1640 and MEGM media supplemented with 3% FBS at 37° C. in a 5% CO₂ humidified incubator. The media bottle was covered with aluminum foil when not in use and aliquots (50-100 mL) were removed and warmed in a 37° C. water bath as needed. Chen et al., 2019. Mouse embryonic fibroblast cells were grown in DMEM with high glucose supplemented with 9% FBS at 34° C. in a 10% CO₂ humidified incubator. Braithwaite et al., 2010; Hu et al., 2004. MCF-7 and MDA-MB-436 cells were grown in RPMI 1640 Medium with GlutaMAX supplement, supplemented with 0.05% insulin, 9% FBS, and 1% antibiotic antimycotic solution at 37° C. in a 5% CO₂ humidified incubator. Amin et al., 2015; Song et al., 2019.

### 2.3.3. Clonogenic Assay for Cell Survival

Approximately 2×10⁵ cells were plated in each well of a 6-well culture plate (well size; 35 mm×18 mm) in medium (1 mL) and kept in a humidified incubator at 37° C. with 5% CO₂. After incubating overnight, cells were subjected to either the vector (50% MeCN in H₂O, 10 μL) or treatment (100× pro-14, 10 μL or 100× Olaparib, 10 μL). After treatment, the medium was removed, and the cells were washed with 1×PBS (2×1 mL). The cells were trypsinized with 0.25% Trypsin-EDTA (0.5 mL in each well, 5 min incubation at 37° C.), washed with the appropriate media supplemented with FBS for the corresponding cell line (1 mL) to quench the trypsin cleavage, and spun down (3,000 RCF×5 min). The medium was removed, and the cells were resuspended in fresh media (10 mL). The single cell suspensions were collected and counted using a TC20 Automated Cell Counter (BIO-RAD). Stock solutions of single cell suspensions were prepared for all untreated and treated cells. For example, 100 cells/mL stock solution of untreated cells were prepared; 500 cells/mL stock solutions of treated cells were prepared. The concentration of stock solution for each sample was chosen based on expected toxicity of the treatment (i.e., higher cell counts for more toxic conditions). The appropriate number of cells for each experiment were seeded in each well of a 6-well plate (well size; 35 mm×18 mm) in 3 mL of medium. The cells were grown in a humidified incubator at 37° C. with 5% CO₂ for 14 days (21 days for UWB1.289 cells). No significant change in media volume was observed after two-three weeks due to evaporation. After 14 days, the growth medium was discarded, and the attached cells were treated with 0.2% w/v aqueous crystal violet solution (1 mL). The excess dye was removed by washing with water twice. The plates were dried and scanned with an HP Scanjet 3970 and colonies were counted using ImageJ (FIJI, free download from NIH). Plating efficiencies (PE) and survival fractions (SF) were calculated as follows: PE=number of colonies/number of cells seeded; SF=PE/PE_{control}.

### 2.3.4. Cell Viability Assays for Mouse Embryonic Fibroblasts (Braithwaite et al., 2010; Hu et al., 2004)

Mouse embryonic fibroblasts (Pol β WT, Pol β−/−) were seeded at a density of ˜3×105 cells/well in 6-well dishes. The following day, cells were exposed to pro-14 (0, 5, 25 μM) or Olaparib (0, 0.5, 1 μM) in medium (1 mL) for 1 h. Control wells were treated with an equal volume of vector (50% MeCN in H₂O). Cells were washed with 1×PBS and fresh medium was added. Dishes were incubated at 34° C. in a 10% CO₂ incubator, until untreated control cells were approximately 80% confluent, which was 5 days. Cells (triplicate wells for each treatment concentration were counted by a cell lysis procedure (described previously in the clonogenic assay procedure), and the results were expressed as the surviving fraction of cells in drug-treated wells relative to control wells.

### 2.3.5 Pol β Knock Down by siRNA (Ray et al., 2018)

UWB1.289 cells (6×106) were seeded with RPMI 1640/MEGM medium (10 mL) into a 100 mm plate prior to transfection so cells would be adherent and ˜80% confluent on the first day of transfection, which was determined by manual inspection. Transfection was completed as follows:
1. Lipofectamine RNAiMAX (60 μL) was diluted in Opti-MEM reduced serum medium to 1 mL.
2. In a separate tube, siRNA (either siNT or siPol β, 10 μM, 60 μL, 600 μmol) was diluted in Opti-MEM (500 μL).
3. The Lipofectamine solution was split (2×500 μL aliquots) and mixed with either siNT or siPol β solution to form the lipid-RNA complex at 25° C. for 5 min.
4. The RPMI 1640/MEGM medium was removed and replaced with Opti-MEM medium (9 mL). After 5 min incubation, the lipid-RNA solution (1 mL) was added to the cells. Cells were incubated at 37° C. in a 5% CO₂ humidified incubator. The following day, steps 1-3 were repeated. and the resulting 2×500 μL solutions were added to the existing cells that had been incubating at 37° C., 5% CO₂ for 24 h. Transfected cells were allowed to incubate for an additional 48 h at 37° C., 5% CO₂ after the second addition of siRNA. After a total of 72 h, cells were harvested (described previously in the clonogenic assay procedure) and reseeded into 6-well plates for a clonogenic assay (described previously). A portion of the transfected cells (2×10⁶) were used to determine knock down efficiency by Western blot.

### 2.3.6 Western Blot Analysis

Cells were trypsinized with 0.25 w/v Trypsin-EDTA (1 mL in each plate (100 mm), 5 min incubation at 37° C.), washed with RPMI 1640/MEGM-FBS (10 mL) to quench the trypsin cleavage, and spun down (3,000 RCF×5 min). The medium was removed, and cells were washed with cold 1×PBS buffer and spun down (2×3000 RCF, 4° C., 5 min) twice. The cell pellet was resuspended in nuclear fractionation lysis buffer (1 mL/10⁷ cells; 20 mM HEPES pH 7.5, 0.25 M sucrose, 1 mM PMSF, 3 mM MgCl₂, 0.2% NP-40, with protease inhibitor). Cells were incubated in lysis buffer on ice for 15 min with occasional mixing by inverting the tube back and forth, after which they were spun down (3,000 RCF, 4° C., 15 min). The supernatant was discarded. The cell pellet was resuspended and incubated in RIPA lysis buffer (0.5 mL/10⁷ cells; 10 mM Tris pH 8.0, 1 mM EDTA, 0.5 mM EGTA, 1% sodium deoxycholate, 0.1% SDS, 140 mM NaCl) on ice for 20 min with occasional mixing by inverting the tube back and forth. The cells were spun down (16,000 RCF, 4° C., 10 min). The supernatant (cell lysate) was transferred to a new tube. The concentration of proteins in cell lysate were determined by Bradford assay. The cell lysate was split into two sets and each portion (20 μg of protein) along with Precision Plus Protein WesternC standard (5 μL) protein ladder was loaded separately onto precast 4-20% SDS-PAGE to visualize the amount of Pol β present or an internal standard (i.e., a-H3). The H3 antibody was used to quantify the relative amounts of H3 in the control and knock down cells to account for any variation in protein(s) loaded onto the gel. The relative amounts of H3 were used to normalize the knock down of Pol β expression observed. Proteins were transferred from the gel to a nitrocellulose membrane (25 V, 1 A, 30 min) using a BioRad Trans-Blot Turbo Transfer system. The membrane was stained with ponceau red stain for 5 min then washed with H₂O until the background was removed. The membrane was cut and separated so one set of samples could be incubated with the Pol β antibody and the other set with H3 antibody. The membranes were blocked with 3% BSA in 1× Tris-buffered saline tween (TBST; 20 mM Tris, 150 mM NaCl, 0.1% Tween) buffer for 30 min at 25° C. while shaking. The membranes were incubated with the primary antibody solution (5 mL, Recombinant Anti-DNA Polymerase beta antibody, 1:1000 Ab in 3% BSA in TBST; Recombinant Anti-Histone H3 antibody, 1:1000 Ab in TBST) at 4° C. overnight. The membranes were washed (3×5 min TBST, 5 mL). The membranes were incubated with secondary antibody solution (Rabbit anti-goat antibody, 5 mL, 1:5000 Ab in 3% BSA in TBST with StrepTactin-HRP conjugate, 0.5 IL) at 25° C. for 1 h. The membranes were washed (3×5 min TBST, 5 mL). Membranes were kept in Trisbuffered saline (TBS; 20 mM Tris, 150 mM NaCl) buffer until incubated with developing reagent (5 mL) for 5 min and scanned using a Typhoon 9410 equipped with chemiluminescence imaging (Medium quality, 600 PVT, 200 pixels).

### Example 3

### Synthesis of Polymerase Inhibitors

Methods for the synthesis of libraries of representative polymerase inhibitors (Scheme 9 (13), as well as the synthesis of the pro-inhibitor (18, Scheme 10) and inhibitor (22, Scheme 11) are provided immediately herein below. In addition, a method has been developed for generated inhibitor libraries containing a slightly longer alkyl chain at which diversification group “R₂” is incorporated (34, Scheme 12).

In some embodiments, the synthesis methods of the chemical library (13) include introducing the C₅-azidomethyl group (3) via a procedure that is reported in the literature. In addition, the 3-functionality (8) is introduced via a cyclonucleoside (7). Finally, the dioxobutane “warhead” component is protected using a photolabile group (15) that is cleaved rapidly upon photolysis at 350 nm using commonly available light sources.

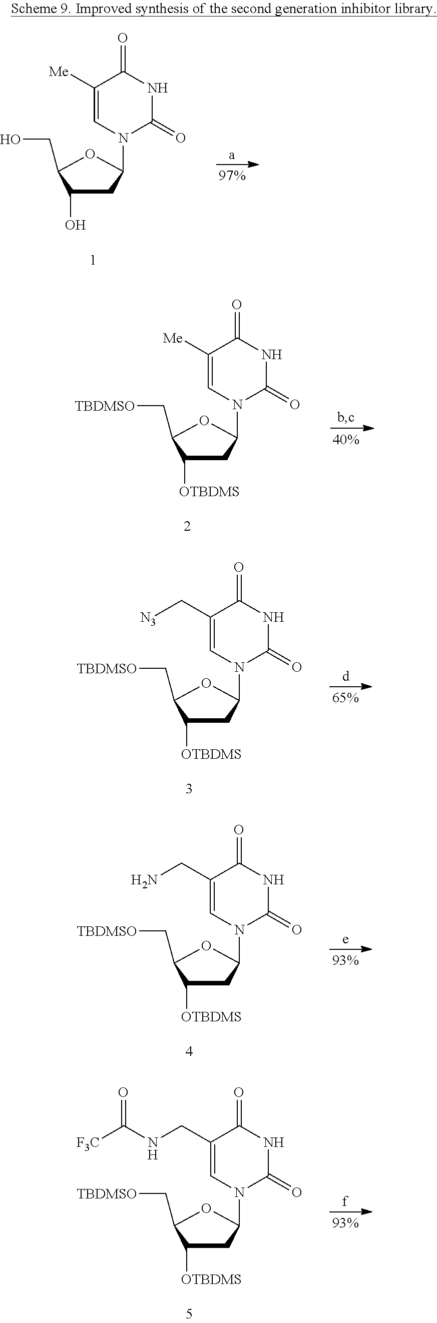

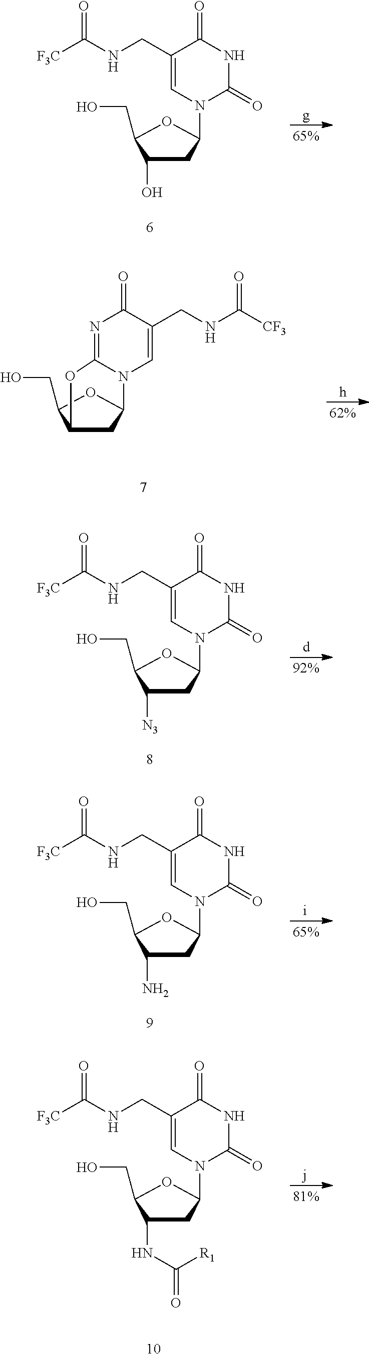

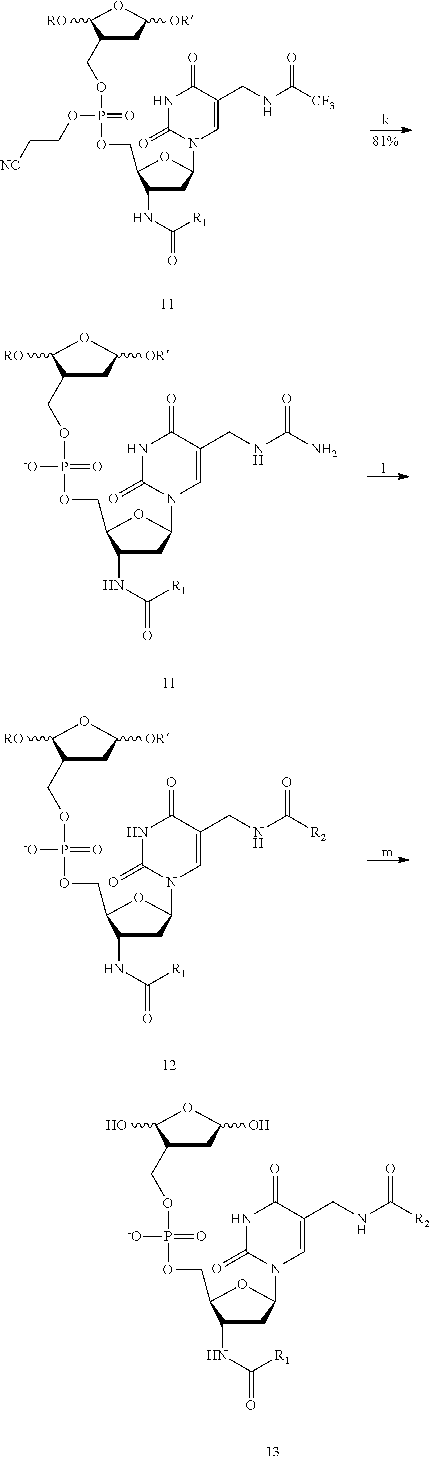

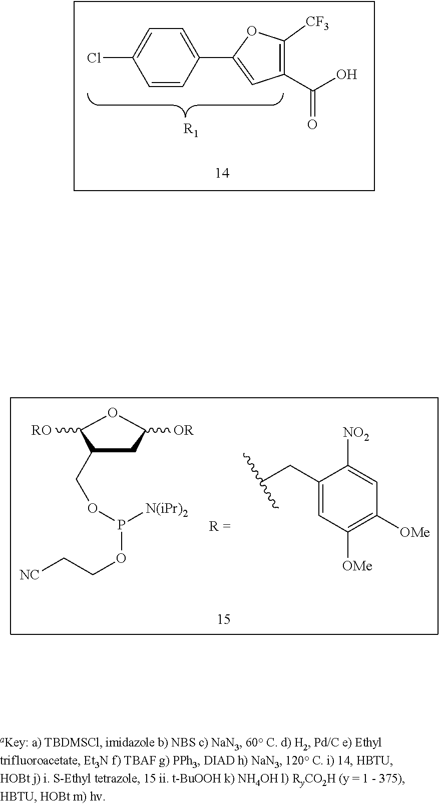

Pro-inhibitor 18 (Scheme 10). which is used in cell experiments, has been prepared on more than 150 mg scale, and can be scaled-up further. One improvement that makes this scale up possible is the preparation of 10 (Scheme 9). Introduction of the C5-methyl diversity element (R₂) using the corresponding N-hydroxysuccinimide (19) also led to greater overall yield.

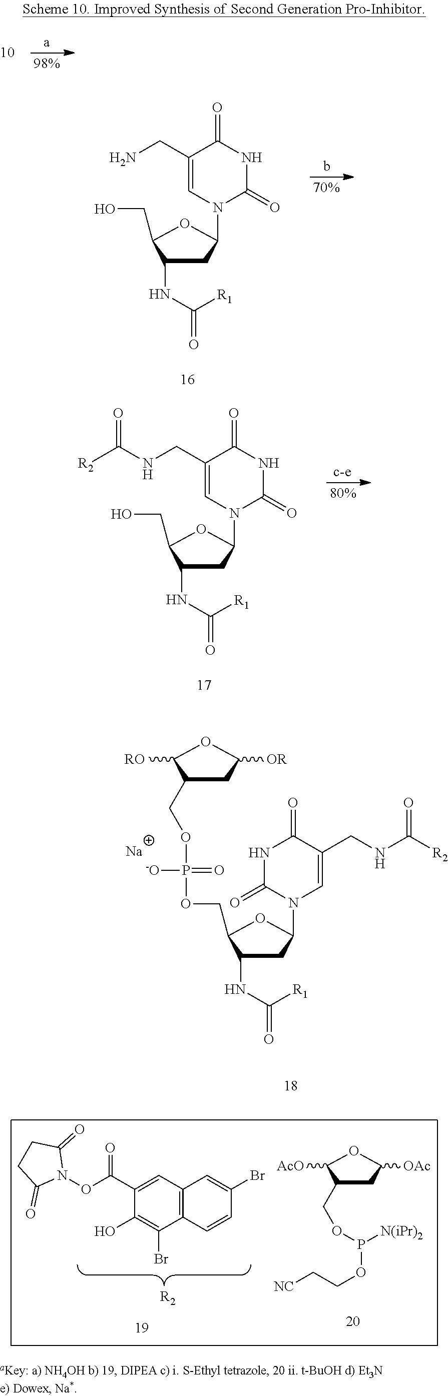

Preparation of the inhibitor (22, Scheme 11) also benefited by the above changes, including photochemical release of the inhibitor.

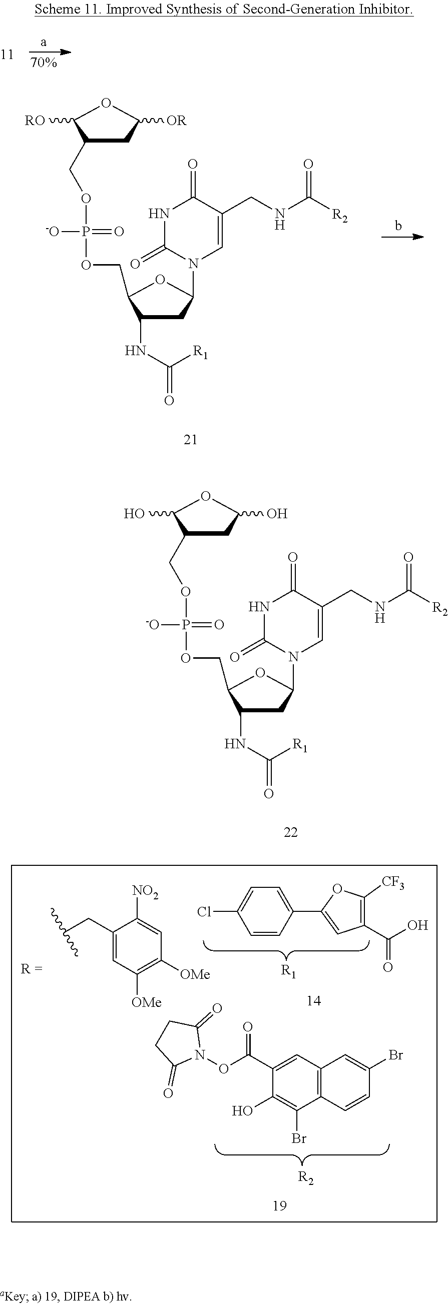

A similar synthetic approach was developed to prepare chemical libraries of inhibitor candidates containing a 3-carbon linker at the C5-position of the pyrimidine (Scheme 12). The purpose of this modification is to provide greater flexibility in probing the space within the enzyme binding site. The synthesis begins from commercially available 5-iodo-2′-deoxyuridine (23), and proceeds through cyclonucleoside 24, which is a key intermediate for introducing the C5-carbon chain and the nucleophile at the C3′-position. The 3-carbon linker is introduced using Pd(0) chemistry and the C3′-nucleophile is introduced via azide, as described above (Scheme 9). Following preparation of 28, the synthesis proceeds similarly to that (

[symbol]

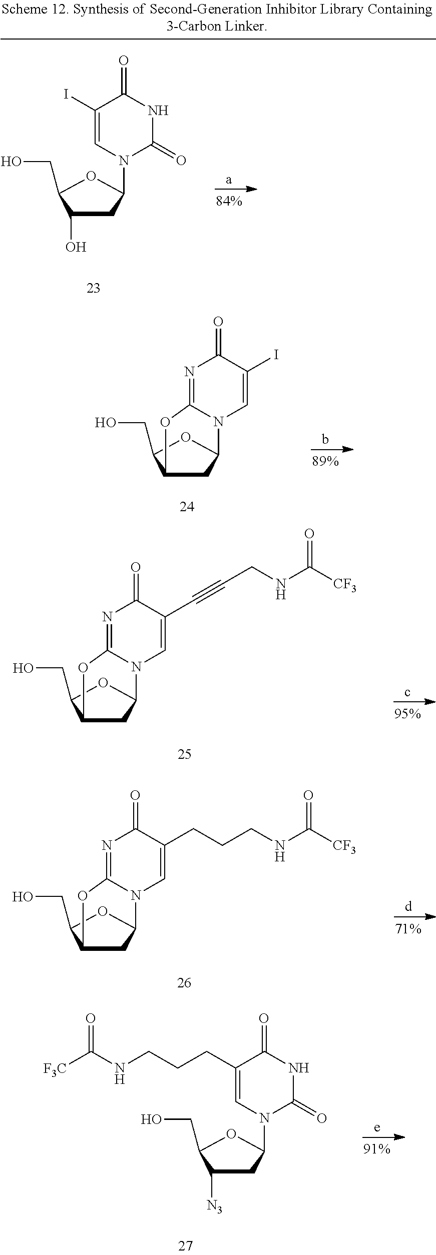

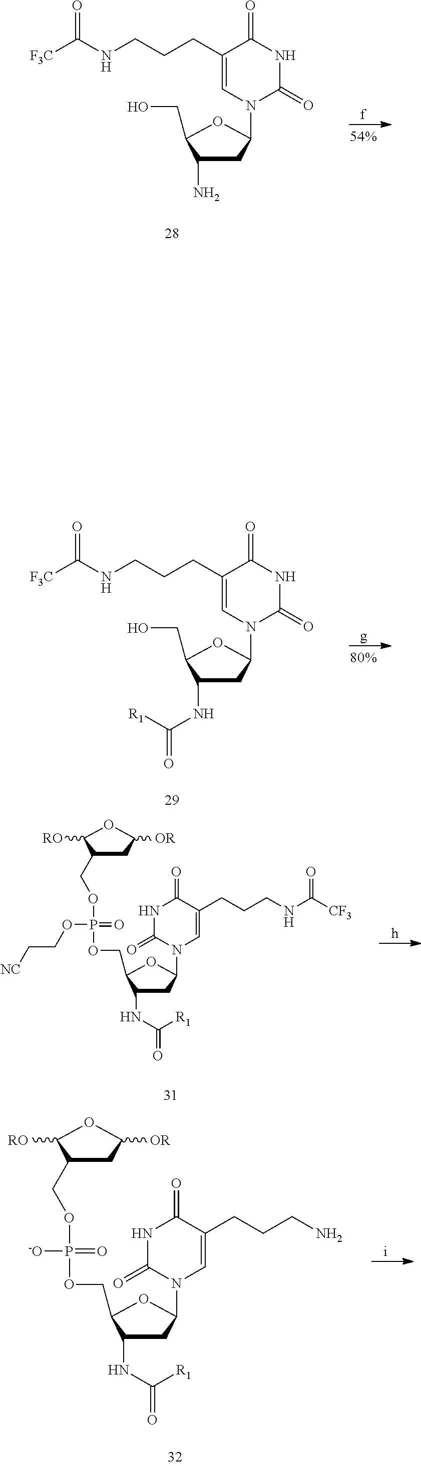

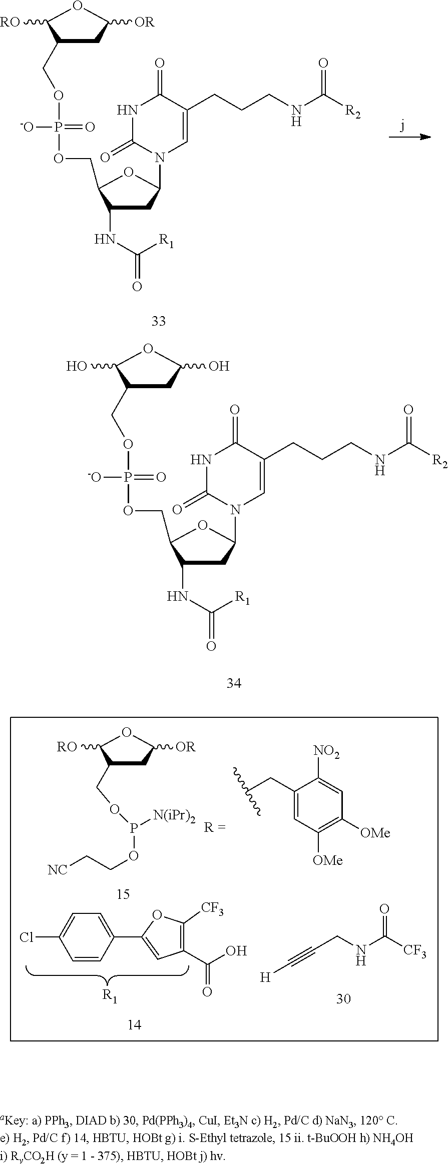

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Zhao, L.; Sumberaz, P. Mitochondrial DNA Damage: Prevalence, Biological Consequence, and Emerging Pathways. Chem. Res. Toxicol. 2020, 33, 2491-2502.
Nelson, B. C.; Dizdaroglu, M. Implications of DNA Damage and DNA Repair on Human Diseases. Mutagenesis 2020, 35, 1-3.
Thompson, P. S.; Cortez, D. New Insights into Abasic Site Repair and Tolerance. DNA Repair 2020, 90, 102866.
Lindahl, T. The Intrinsic Fragility of DNA. Angew. Chem. Int. Ed. 2016, 55, 8528-8534.
Wheeler, D. A.; Takebe, N.; Hinoue, T.; Hoadley, K. A.; Cardenas, M. F.; Hamilton, A. M.; Laird, P. W.; Wang, L.; Johnson, A.; Dewal, N.; Miller, V.; Pineyro, D.; Castro de Moura, M.; Esteller, M.; Shen, H.; Zenklusen, J. C.; Tamuzzer, R.; McShane, L. M.; Tricoli, J. V.; Williams, P. M.; Lubensky, I.; O'Sullivan-Coyne, G.; Kohn, E. C.; Little, R. F.; White, J.; Malik, S.; Harris, L.; Well, C.; Chen, A. P.; Karlovich, C.; Rodgers, B.; Shankar. L.; Jacobs, P.; Nolan, T.; Hu, J.; Muzny, D. M.; Doddapaneni, H.; Korchina, V.; Gastier-Foster, J.; Bowen, J.; Leraas, K.; Edmondson, E. F.; Doroshow, J. H.; Conley, B. A.; Ivy, S. P.; Staudt, L. M. Molecular Features of Cancers Exhibiting Exceptional Responses to Treatment. Cancer Cell 2020, Ahead of Print. (doi: 10.1016/j.ccell.2020.10.015) Farmer, H.; McCabe, N.; Lord, C. J.; Tutt, A. N. J.; Johnson, D. A.; Richardson, T.
B.; Santarosa, M.; Dillon, K. J.; Hickson, I.; Knights, C.; Martin, N. M. B.; Jackson, S. P.; Smith, G. C. M.; Ashworth, A. Targeting the DNA Repair Defect in BRCA Mutant Cells as a Therapeutic Strategy. Nature 2005, 434, 917-921.
Rottenberg, S.; Jaspers, J. E.; Kersbergen, A.; van der Burg, E.; Nygren, A. O. H.; Zander, S. A. L.; Derksen, P. W. B.; de Bruin, M.; Zevenhoven, J.; Lau, A.; Boulter, R.; Cranston, A.; Connor, M. J.; Martin, N. M. B.; Borst, P.; Jonkers, J. High Sensitivity of Brca1-Deficient Mammary Tumors to the PARP Inhibitor Azd2281 Alone and in Combination with Platinum Drugs. Proc. Natl. Acad. Sci. USA 2008, 105, 17079.
Lord, C.; Ashworth, S. PARP Inhibitors: Synthetic Lethality in the Clinic. Science 2017, 355, 1152-1158.
Tahara, Y.-K.; Kietrys, A. M.; Hebenbrock, M.; Lee, Y.; Wilson, D. L.; Kool, E. T. Dual Inhibitors of 8-Oxoguanine Surveillance by Ogg1 and Nudt1. ACS Chem. Biol. 2019, 14, 2606-2615.
Tahara, Y.-K.; Auld, D.; Ji, D.; Beharry, A. A.; Kietrys, A. M.; Wilson, D. L.; Jimenez, M.; King, D.; Nguyen, Z.; Kool, E. T. Potent and Selective Inhibitors of 8-Oxoguanine DNA Glycosylase. J. Am. Chem. Soc. 2018, 140, 2105-2114.
Visnes, T.; Cázares-Kömer, A.; Hao, W.; Wallner, O.; Masuyer, G.; Loseva, O.; Mortusewicz, O.; Wiita, E.; Samo, A.; Manoilov, A.; Astorga-Wells, J.; Jemth, A.-S.; Pan, L.; Sanjiv, K.; Karsten, S.; Gokturk, C.; Grube, M.; Homan, E. J.; Hanna, B. M. F.; Paulin, C. B. J.; Pham, T.; Rasti, A.; Berglund, U. W.; von Nicolai, C.; Benitez-Buelga, C.; Koolmeister, T.; Ivanic, D.; Iliev, P.; Scobie, M.; Krokan, H. E.; Baranczewski, P.; Artursson, P.; Altun, M.; Jensen, A. J.; Kalderen, C.; Ba, X.; Zubarev, R. A.; Stenmark, P.; Boldogh, I.; Helleday, T. Small-Molecule Inhibitor of Ogg1 Suppresses Proinflammatory Gene Expression and Inflammation. Science 2018, 362, 834.
Donley, N.; Jaruga, P.; Coskun, E.; Dizdaroglu, M.; McCullough, A. K.; Lloyd, R. S. Small Molecule Inhibitors of 8-Oxoguanine DNA Glycosylase-1 (Ogg1). ACS Chem. Biol. 2015, 10, 2334-2343.
Huang, H.; Stivers, J. T.; Greenberg, M. M. Competitive Inhibition of Uracil DNA Glycosylase by a Modified Nucleotide Whose Triphosphate Is a Substrate for DNA Polymerase. J Am. Chem. Soc. 2009, 131, 1344-1345.
Rai, G.; Vyjayanti, V. N.; Dorjsuren, D.; Simeonov, A.; Jadhav, A.; Wilson, D. M.; Maloney, D. J. Synthesis, Biological Evaluation, and Structure-Activity Relationships of a Novel Class of Apurinic/Apyrimidinic Endonuclease 1 Inhibitors. J. Med. Chem. 2012, 55, 3101-3112.
Dorjsuren, D.; Kim, D.; Vyjayanti, V. N.; Maloney, D. J.; Jadhav, A.; Wilson, D. M., III; Simeonov, A. Diverse Small Molecule Inhibitors of Human Apurinic/Apyrimidinic Endonuclease Ape1 Identified from a Screen of a Large Public Collection. PLoS One 2012, 7, e47974.
Wojtaszek, J. L.; Chatterjee, N.; Najeeb, J.; Ramos, A.; Lee, M.; Bian, K.; Xue, J. Y.; Fenton, B. A.; Park, H.; Li, D.; Hemann, M. T.; Hong, J.; Walker, G. C.; Zhou, P. A Small Molecule Targeting Mutagenic Translesion Synthesis Improves Chemotherapy. Cell 2019, 178, 152-159.e111.
Gowda, A. S. P.; Suo, Z.; Spratt, T. E. Honokiol Inhibits DNA Polymerases B and A and Increases Bleomycin Sensitivity of Human Cancer Cells. Chem. Res. Toxicol. 2017, 30, 715-725.
Strittmatter, T.; Bareth, B.; Immel, T. A.; Huhn, T.; Mayer, T. U.; Marx, A. Small Molecule Inhibitors of Human DNA Polymerase 1. ACS Chem. Biol. 2011, 6, 314-319.
Strittmatter, T.; Brockmann, A.; Pott, M.; Hantusch, A.; Brunner, T.; Marx, A. Expanding the Scope of Human DNA Polymerase 1 and β Inhibitors. ACS Chem. Biol. 2014, 9, 282-290.
Jaiswal, A. S.; Panda, H.; Law, B. K.; Sharma, J.; Jani, J.; Hromas, R.; Narayan, S. Nsc666715 and Its Analogs Inhibit Strand-Displacement Activity of DNA Polymerase B and Potentiate Temozolomide-Induced DNA Damage, Senescence and Apoptosis in Colorectal Cancer Cells. PLoS One 2015, 10, e0123808.
Arian, D.; Hedayati, M.; Zhou, H.; Bilis, Z.; Chen, K.; DeWeese, T. L.; Greenberg, M. M. Irreversible Inhibition of DNA Polymerase B by Small-Molecule Mimics of a DNA Lesion. J. Am. Chem. Soc. 2014, 136, 3176-3183.
Paul, R.; Banerjee, S.; Greenberg, M. M. Synergistic Effects of an Irreversible DNA Polymerase Inhibitor and DNA Damaging Agents on Hela Cells. ACS Chem. Biol. 2017, 12, 1576-1583.
Paul, R.; Banerjee, S.; Greenberg, M. M. Synergistic Effects of an Irreversible DNA Polymerase Inhibitor and DNA Damaging Agents on Hela Cells. ACS Chem. Biol. 2018, 13, 832.
Goellner, E. M.; Svilar, D.; Almeida, K. H.; Sobol, R. W. Targeting DNA Polymerase Q for Therapeutic Intervention. Curr. Mol. Pharmacol. 2012, 5, 68-87.
Friedberg, E. C.; Walker, G., C.; Siede, W.; Wood, R. D.; Schultz, R. A.; Ellenberger, T. DNA Repair and Mutagenesis; 2nd ed.; ASM Press:Washington, D.C., 2006.
Wu, S.; Beard, W. A.; Pedersen, L. G.; Wilson, S. H. Structural Comparison of DNA Polymerase Architecture Suggests a Nucleotide Gateway to the Polymerase Active Site. Chem. Rev. 2014, 114, 2759-2774.
Beard, W. A.; Wilson, S. H. Structure and Mechanism of DNA Polymerase B. Biochemistry 2014, 53, 2768-2780.
Prasad, R.; ça\check{g}layan, M.; Dai, D.-P.; Nadalutti, C. A.; Zhao, M.-L.; Gassman, N. R.; Janoshazi, A. K.; Stefanick, D. F.; Horton, J. K.; Krasich, R.; Longley, M. J.; Copeland, W. C.; Griffith, J. D.; Wilson, S. H. DNA Polymerase B: A Missing Link of the Base Excision Repair Machinery in Mammalian Mitochondria. DNA Repair 2017, 60, 77-88.
Sykora, P.; Kanno, S.; Akbari, M.; Kulikowicz, T.; Baptiste, B. A.; Leandro, G. S.; Lu, H.; Tian, J.; May, A.; Becker, K. A.; Croteau, D. L.; Wilson, D. M.; Sobol, R. W.; Yasui, A.; Bohr, V. A. DNA Polymerase Beta Participates in Mitochondrial DNA Repair. Mol. & Cell Biol. 2017, 37, e00237-00217.
Ray, S.; Breuer, G.; DeVeaux, M.; Zelterman, D.; Bindra, R.; Sweasy, J. B. DNA Polymerase Beta Participates in DNA End-Joining. Nucleic Acids Res. 2018, 46, 242-255.
Donigan, K. A.; Sun, K.-w.; Nemec, A. A.; Murphy, D. L.; Cong, X.; Northrup, V.; Zelterman, D.; Sweasy, J. B. Human Polβ Gene Is Mutated in High Percentage of Colorectal Tumors. J Biol. Chem. 2012, 287, 23830-23839.
Nickoloff, J. A.; Jones, D.; Lee, S.-H.; Williamson, E. A.; Hromas, R. Drugging the Cancers Addicted to DNA Repair. J. National Cancer Institute 2017, 109, djx059.
Guan, L.; Greenberg, M. M. Irreversible Inhibition of DNA Polymerase β by an Oxidized Abasic Lesion. J. Am. Chem. Soc. 2010, 132, 5004-5005.
Guan, L.; Bebenek, K.; Kunkel, T. A.; Greenberg, M. M. Inhibition of Short Patch and Long Patch Base Excision Repair by an Oxidized Abasic Site. Biochemistry 2010, 49, 9904-9910.
Jacobs, A. C.; Kreller, C. R.; Greenberg, M. M. Long Patch Base Excision Repair Compensates for DNA Polymerase β Inactivation by the C4′-Oxidized Abasic Site. Biochemistry 2011, 50, 136-143.
Garcia-Diaz, M.; Bebenek, K.; Kunkel, T. A.; Blanco, L. Identification of an Intrinsic 5′-Deoxyribose-5-Phosphate Lyase Activity in Human DNA Polymerase A. J. Biol. Chem. 2001, 276, 34659-34663.
Braithwaite, E. K.; Kedar, P. S.; Stumpo, D. J.; Bertocci, B.; Freedman, J. H.; Samson, L. D.; Wilson, S. H. DNA Polymerases β and 1 Mediate Overlapping and Independent Roles in Base Excision Repair in Mouse Embryonic Fibroblasts. PLoS One 2010, 5, e12229.
Braithwaite, E. K.; Prasad, R.; Shock, D. D.; Hou, E. W.; Beard, W. A.; Wilson, S. H. DNA Polymerase Lambda Mediates a Back-up Base Excision Repair Activity in Extracts of Mouse Embryonic Fibroblasts. J Biol. Chem. 2005, 280, 18469-18475.
Stevens, A. J.; Guan, L.; Bebenek, K.; Kunkel, T. A.; Greenberg, M. M. DNA Polymerase β Inactivation by Oxidized Abasic Sites. Biochemistry 2013, 52, 975-983.
Dorjsuren, D.; Wilson, D. M.; Beard, W. A.; McDonald, J. P.; Austin, C. P.; Woodgate, R.; Wilson, S. H.; Simeonov, A. A Real-Time Fluorescence Method for Enzymatic Characterization of Specialized Human DNA Polymerases. Nucleic Acids Res. 2009, 37, e128-e128.
Zhang, D.; Seeling, G. Dynamic DNA Nanotechnology Using Strand-Displacement Reactions. Nature Chem. 2011, 3, 103-113.
Olson, X.; Kotani, S.; Yurke, B.; Graugnard, E.; Hughes, W. Kinetics of DNA Strand Displacement Systems with Locked Nucleic Acids. J. Phys. Chem. B. 2017, 121, 2594-2602.
Sawaya, M. R.; Pelletier, H.; Kumar, A.; Wilson, S. H.; Kraut, J. Crystal Structure of Rat DNA Polymerase Beta: Evidence for a Common Polymerase Mechanism. Science 1994, 264, 1930-1935.
Yang, L.; Beard, W. A.; Wilson, S. H.; Broyde, S.; Schlick, T. Highly Organized but Pliant Active Site of DNA Polymerase β: Compensatory Mechanisms in Mutant Enzymes Revealed by Dynamics Simulations and Energy Analyses. Biophys. J. 2004, 86, 3392-3408.
Sawaya, M.; Prasad, R.; Wilson, S.; Kraut, J.; Pelletier, H. Crystal Structures of Human DNA Polymerase Beta Complexed with Gapped and Nicked DNA: Evidence for an Induced Fit Mechanism. Biochemistry 1997, 36, 11205-11215.
Laverty, D. J.; Mortimer, I. P.; Greenberg, M. M. Mechanistic Insight through Irreversible Inhibition: DNA Polymerase Θ Uses a Common Active Site for Polymerase and Lyase Activities. J. Am. Chem. Soc. 2018, 140, 9034-9037.
Garcia-Diaz, M.; Dominguez, O.; Lupez-Fernndez, L. A.; de Lera, L. T.; Saniger, M. L.; Ruiz, J. F.; Pirraga, M.; Garcia-Ortiz, M. J.; Kirchhoff, T.; del Mazo, J. S.; Bernad, A.; Blanco, L. DNA Polymerase Lambda (Pol λ), a Novel Eukaryotic DNA Polymerase with a Potential Role in Meiosis. J. Mol. Biol. 2000, 301, 851-867.
Fu, D.; Calvo, J. A.; Samson, L. D. Balancing Repair and Tolerance of DNA Damage Caused by Alkylating Agents. Nat. Rev. Cancer 2012, 12, 104-120.
   Cuesta, A.; Taunton, J. Lysine-Targeted Inhibitors and Chemoproteomic Probes. Ann. Rev. Biochem. 2019, 88, 365-381.
Wan, X.; Yang, T.; Cuesta, A.; Pang, X.; Balius, T. E.; Irwin, J. J.; Shoichet, B. K.; Taunton, J. Discovery of Lysine-Targeted Eif4e Inhibitors through Covalent Docking. J. Am. Chem. Soc. 2020, 142, 4960-4964.
Pettinger, J.; Jones, K.; Cheeseman, M. D. Lysine-Targeting Covalent Inhibitors. Angew. Chem. Int. Ed. 2017, 56, 15200-15209.
Sutanto, F.; Konstantinidou, M.; Domling, A. Covalent Inhibitors: A Rational Approach of Drug Discovery. RSC Med. Chem. 2020, 11, 876-884.
Prasad, R.; Kumar, A.; Widen, S.; Casas-Finet, J.; Wilson, S. Identification of Residues in the Single-Stranded DNA-Binding Site of the 8-KDa Domain of Rate DNA Polymerase β by UV Cross-Linking. J. Biol. Chem. 1993, 268, 22746-22755.
Beard, W.; Wilson, S. Purification and Domain-Mapping of Mammalian DNA Polymerase β. Methods Enzymol. 1995, 262, 98-107.
Fiala, K. A.; Abdel-Gawad, W.; Suo, Z. Pre-Steady-State Kinetic Studies of the Fidelity and Mechanism of Polymerization Catalyzed by Truncated Human DNA Polymerase λ†. Biochemistry 2004, 43 (21), 6751-6762.
Xu, P.; Chen, D.; Xi, J.; Yao, Z. Short Protecting Group-free Syntheses of Camptothecin and 10-Hydroxycamptothecin Using Cascade Methodologies. Chem.—Asian J. 2015, 10 (4), 976-981.
Handlon, A.; Oppenheimer, N. Thiol Reduction of 3′-Azidothymidine to 3′-Aminothymidine: Kinetics and Biomedical Implications. Pharm Res 1998, 5, 297-299.
Vanheusden, V.; Munier-Lehmann, H.; Froeyen, M.; Dugue, L.; Heyerick, A.; Keukeleire, D. D.; Pochet, S.; Busson, R.; Herdewijn, P.; Calenbergh, S. V. 3′-C-Branched-Chain-Substituted Nucleosides and Nucleotides as Potent Inhibitors of Mycobacterium Tuberculosis Thymidine Monophosphate Kinase. J Med Chem. 2003 Aug. 28; 46(18):3811-21.
Vemekar, S. K. V.; Qiu, L.; Zhang, J.; Kankanala, J.; Li, H.; Geraghty, R. J.; Wang, Z. 5′-Silylated 3′-1,2,3-Triazolyl Thymidine Analogues as Inhibitors of West Nile Virus and Dengue Virus. J. Med. Chem. 2015, 58 (9), 4016-4028.
He, P.; Yang, W. Template and Primer Requirements for DNA Pol θ-Mediated End Joining. Proc. Natl. Acad. Sci. 2018, 115 (30), 7747-7752.
Hu, H. Y.; Horton, J. K.; Gryk, M. R.; Prasad, R.; Naron, J. M.; Sun, D. A.; Hecht, S. M.; Wilson, S. H.; Mullen, G. P. Identification of Small Molecule Synthetic Inhibitors of DNA Polymerase β by NMR Chemical Shift Mapping. J. Biol. Chem. 2004, 279 (38), 39736-39744.
Higgins, G. S.; Boulton, S. J. Beyond PARP−Polθ as an Anticancer Target. Science 2018, 359, 1217.
Ceccaldi, R.; Liu, J. C.; Amunugama. R.; Hajdu, I.; Primack, B.; Petalcorin, M. I. R.; O'Connor, K. W.; Konstantinopoulos, P. A.; Elledge, S. J.; Boulton, S. J.; Yusufzai, T.; D'Andrea, A. D. Homologous-Recombination-Deficient Tumours Are Dependent on Polθ-Mediated Repair. Nature 2015, 518, 258-262.
Hewitt, G.; Borel, V.; Segura-Bayona, S.; Takaki, T.; Ruis, P.; Bellelli, R.; Lehmann, L. C.; Sommerova, L.; Vancevska, A.; Tomas-Loba, A.; Zhu, K.; Cooper, C.; Fugger, K.; Patel, H.; Goldstone, R.; Schneider-Luftman, D.; Herbert, E.; Stamp, G.; Brough, R.; Pettitt, S.; Lord, C. J.; West, S. C.; Ahel, I.; Ahel, D.; Chapman, J. R.; Deindl, S.; Boulton, S. J. Defective Alc1 Nucleosome Remodeling Confers PARPi Sensitization and Synthetic Lethality with HRD. Mol. Cell 2021, 81, 767-783.e711.
Ali, R.; Alblihy, A.; Miligy, I. M.; Alabdullah, M. L.; Alsaleem, M.; Toss, M. S.; Algethami, M.; Abdel-Fatah, T.; Moseley, P.; Chan, S.; Mongan, N. P.; Narayan, S.; Rakha, E. A.; Madhusudan, S. Molecular Disruption of DNA Polymerase B for Platinum Sensitisation and Synthetic Lethality in Epithelial Ovarian Cancers. Oncogene 2021, doi: 10.1038/s41388-41021-01710-y.
Beard, W. A.; Wilson, S. H. Structure and Mechanism of DNA Polymerase β. Chem. Rev. 2006, 106, 361-382.
Roy, R.; Chun, J.; Powell, S. N. Brca1 and Brca2: Different Roles in a Common Pathway of Genome Protection. Nature Reviews Cancer 2012, 12, 68-78.
O'Neil, N. J.; Bailey, M. L.; Hieter, P. Synthetic Lethality and Cancer. Nature Rev. Gen. 2017, 18, 613-623.
Huang, A.; Garraway, L. A.; Ashworth, A.; Weber, B. Synthetic Lethality as an Engine for Cancer Drug Target Discovery. Nature Rev. Drug Discovery 2020, 19, 23-38.
Baptiste, B. A.; Baringer, S. L.; Kulikowicz, T.; Sommers, J. A.; Croteau, D. L.; Brosh, R. M.; Bohr, V. A. DNA Polymerase B Outperforms DNA Polymerase F in Key Mitochondrial Base Excision Repair Activities. DNA Repair 2021, 99, 103050.
Barakat, K. H.; Gajewski, M. M.; Tuszynski, J. A. DNA Polymerase Beta (Pol (3) Inhibitors: A Comprehensive Overview. Drug Discovery Today 2012, 17, 913-920.
Chen, S.-H.; Yu, X. Targeting DePARylation Selectively Suppresses DNA Repair-Defective and PARP Inhibitor-Resistant Malignancies. Science 2019, 5, eaav4340.
Amin, O.; Beauchamp, M.-C.; Nader, P.; Laskov, I.; Iqbal, S.; Philip, C.-A.; Yasmeen, A.; Gotlieb, W. Suppression of Homologous Recombination by Insulin-like Growth Factor-1 Inhibition Sensitizes Cancer Cells to PARP Inhibitors. BMC Cancer 2015, 15, 817-.
Song, Z.; Zhang, X.; Lin, Y.; Wei, Y.; Liang, S.; Dong, C. LINC01133 Inhibits Breast Cancer Invasion and Metastasis by Negatively Regulating SOX4 Expression through EZH2. Journal Cell. Mol. Med. 2019, 23, 7554-7565.
Yuhas, S. C.; Laverty, D. J.; Lee, H.; Greenberg, M. M. Selective Inhibition of DNA Polymerase β by a Covalent Inhibitor, submitted for publication.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 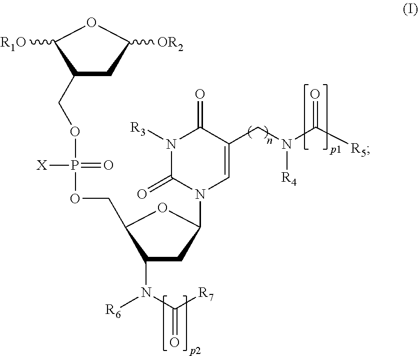
wherein:
n is an integer selected from 1, 2, 3, 4, 5, 6, 7, and 8;
X is selected from the group consisting of substituted or unsubstituted C₁-C₆ alkyl, O—, S— and —OR₈, wherein R₈ is substituted or unsubstituted C₁-C₆ alkyl;
p1 and p2 are each independently an integer selected from 0 or 1;
R₁ and R₂ are each independently selected from the group consisting of hydrogen and —C(═O)—R₉, wherein R₉ is selected from the group consisting of substituted or unsubstituted C₁-C₆ alkyl, heteroalkyl, cycloalkyl, cycloheteroalkyl, aryl, heteroaryl, arylalkyl, and heteroarylalkyl;
R₃, R₄, and R₆ are each independently H or C₁-C₆ alkyl;
R₅ and R₇ are each independently derived from a carboxylic acid-containing moiety selected from the group consisting of substituted or unsubstituted alkyl, heteroalkyl, cycloalkyl, cycloheteroalkyl, aryl, heteroaryl, arylalkyl, heteroarylalkyl, multicyclic aliphatic ring systems, multicyclic aromatic ring systems, fused aliphatic ring systems, fused aromatic ring systems, and combinations thereof; and
pharmaceutically acceptable salts thereof.

2. The compound of claim 1, wherein R₁ and R₂ are each independently H or —(C═O)—CH₃.

3. The compound of claim 1, wherein R₅ and R₇ are each independently selected from the group consisting of:

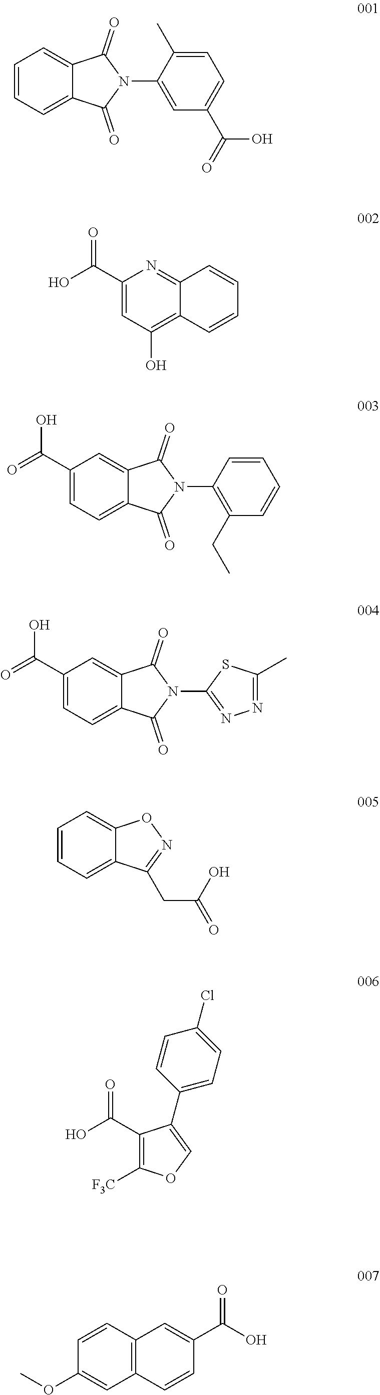

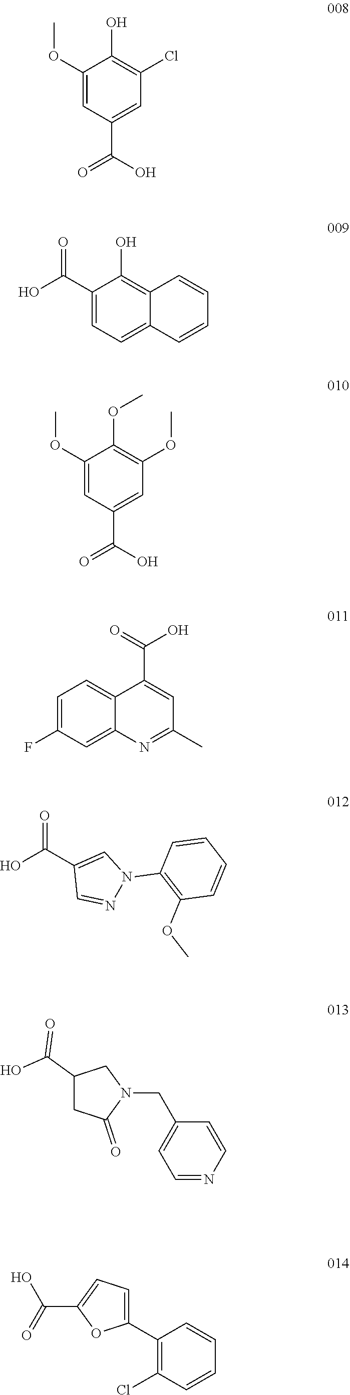

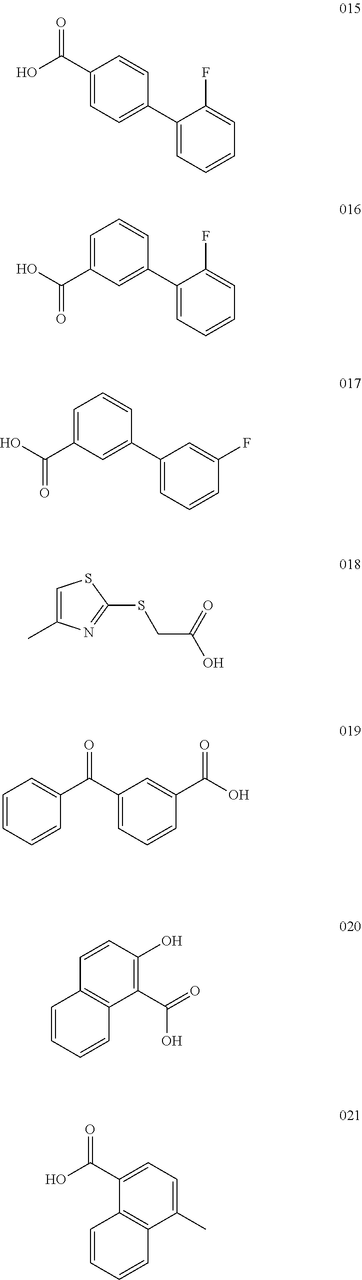

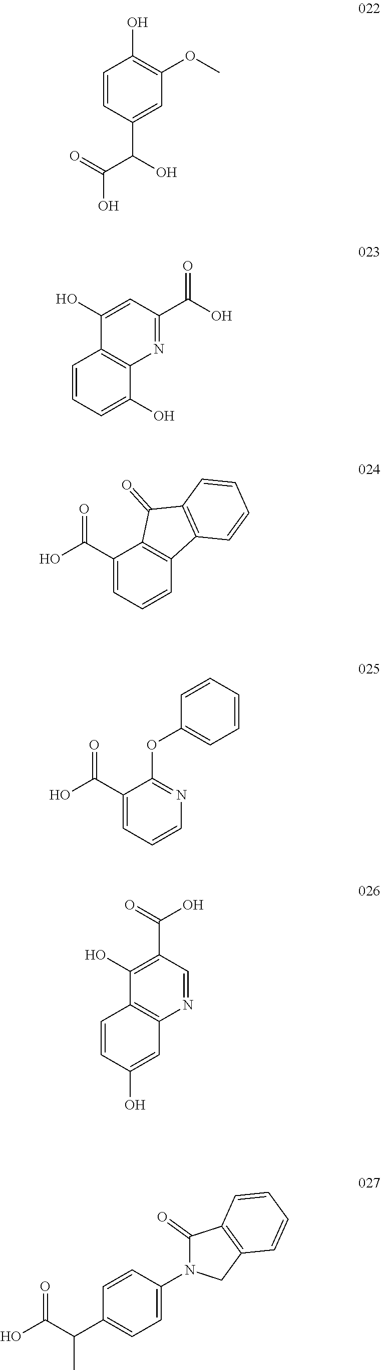 [SMILES: COc1cc(C(O)C(=O)O)ccc1O ; O=C(O)c1cc(O)c2cccc(O)c2n1 ; O=C(O)c1cccc2c1C(=O)c1ccccc1-2 ; O=C(O)c1cccnc1Oc1ccccc1 ; O=C(O)c1cnc2cc(O)ccc2c1O ; CC(C(=O)O)c1ccc(N2Cc3ccccc3C2=O)cc1]

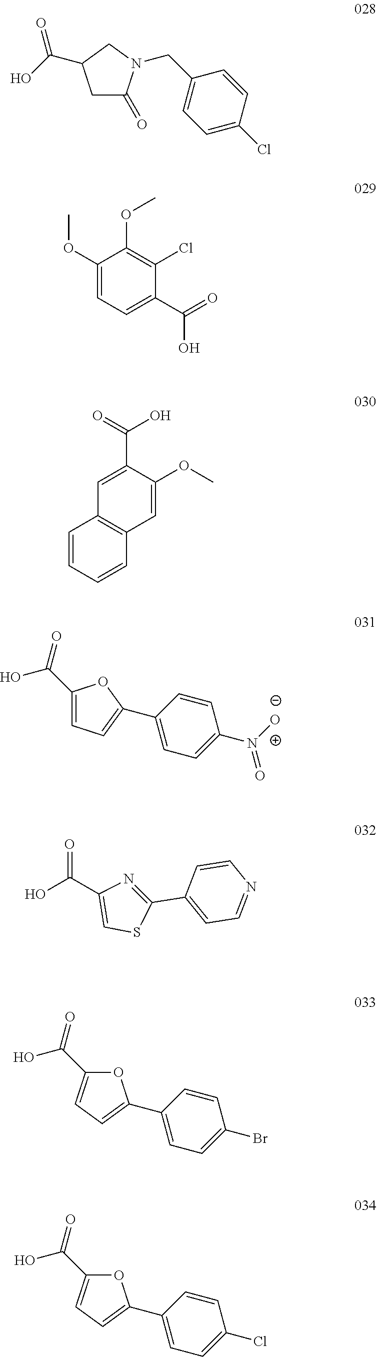

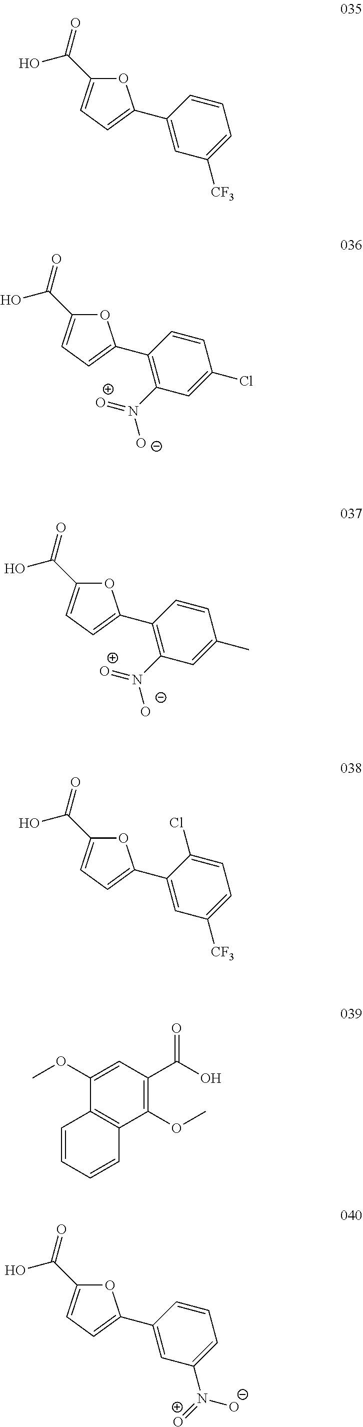

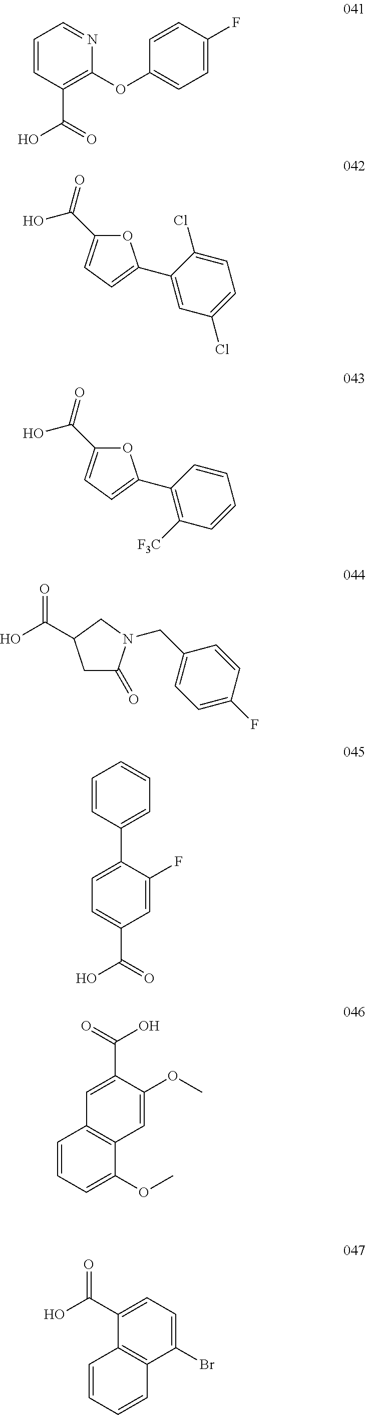

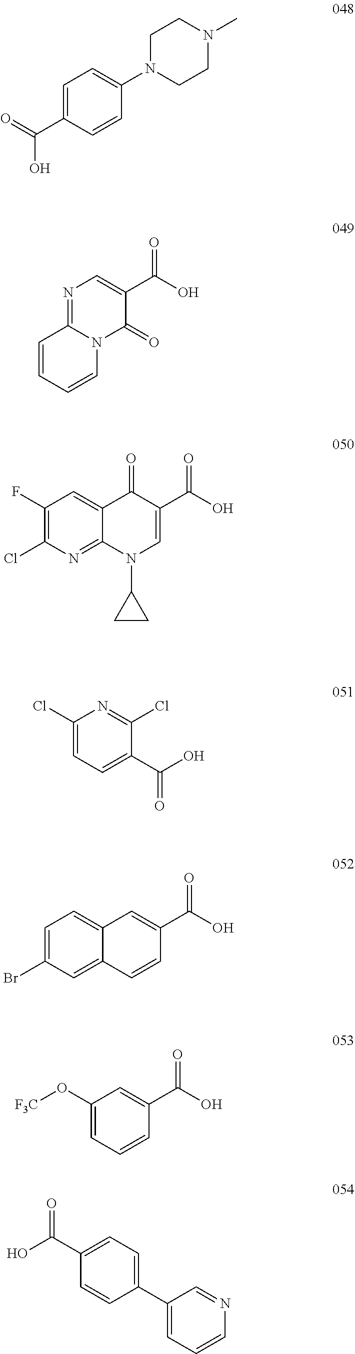

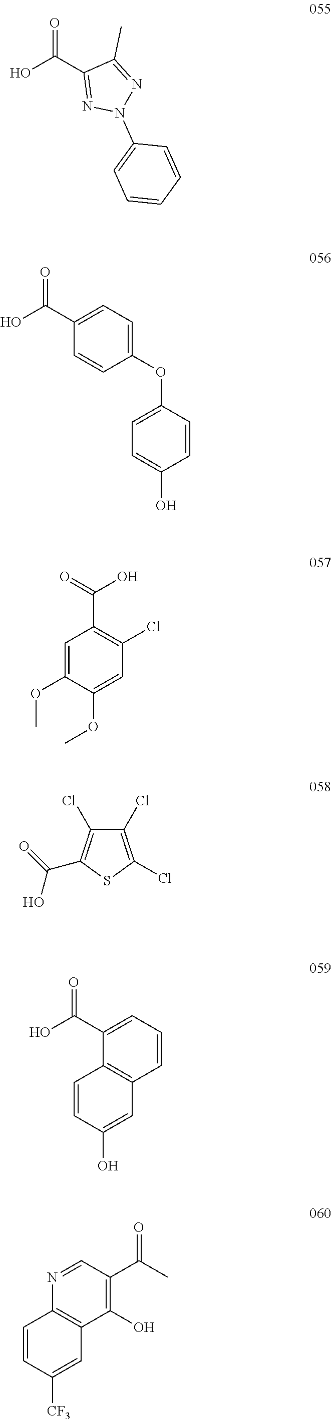 [SMILES: Cc1nn(-c2ccccc2)nc1C(=O)O ; O=C(O)c1ccc(Oc2ccc(O)cc2)cc1 ; COc1cc(Cl)c(C(=O)O)cc1OC ; O=C(O)c1sc(Cl)c(Cl)c1Cl ; O=C(O)c1cccc2cc(O)ccc12 ; CC(=O)c1cnc2ccc(C(F)(F)F)cc2c1O]

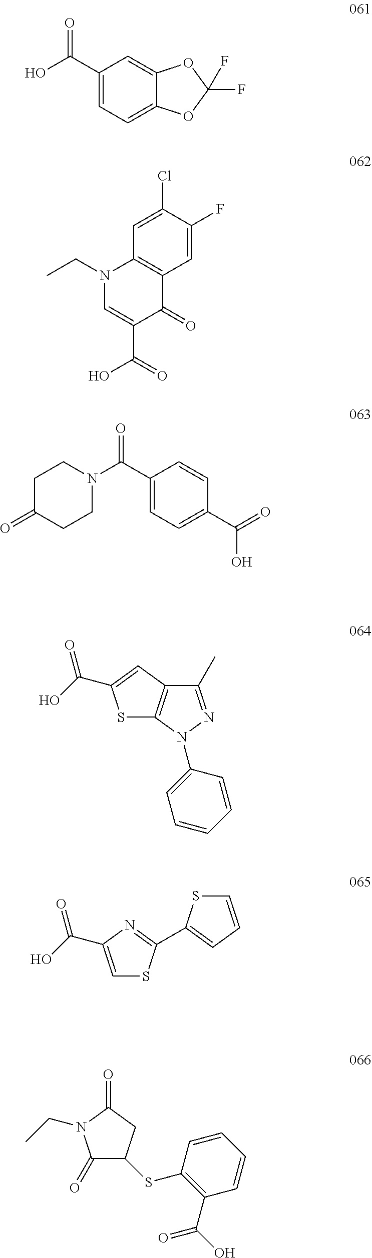 [SMILES: O=C(O)c1ccc2c(c1)OC(F)(F)O2 ; CCn1cc(C(=O)O)c(=O)c2cc(F)c(Cl)cc21 ; O=C1CCN(C(=O)c2ccc(C(=O)O)cc2)CC1 ; Cc1nn(-c2ccccc2)c2sc(C(=O)O)cc12 ; O=C(O)c1csc(-c2cccs2)n1 ; CCN1C(=O)CC(Sc2ccccc2C(=O)O)C1=O]

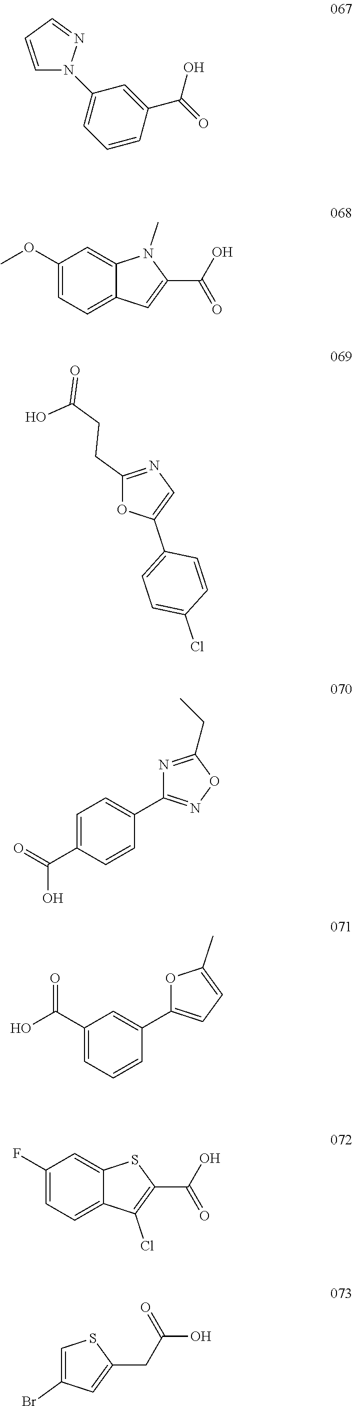

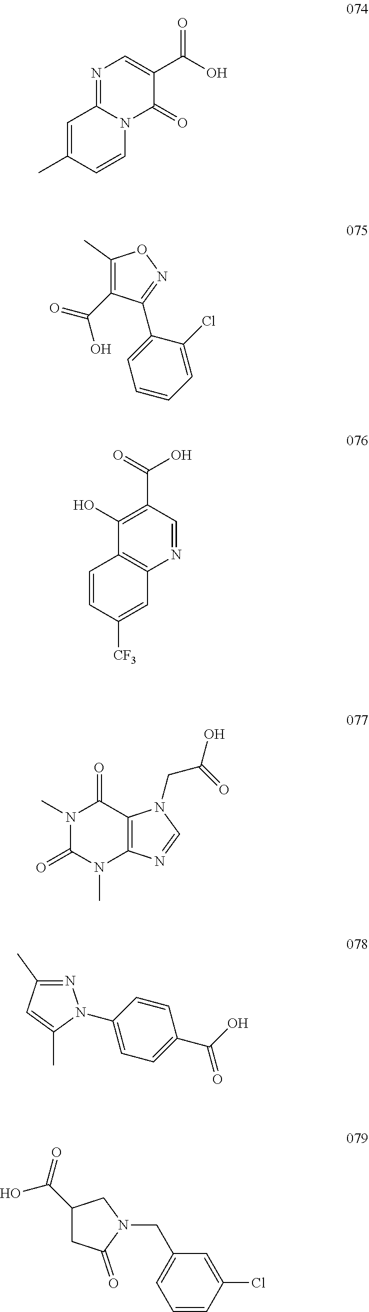 [SMILES: Cc1ccn2c(=O)c(C(=O)O)cnc2c1 ; Cc1onc(-c2ccccc2Cl)c1C(=O)O ; O=C(O)c1cnc2cc(C(F)(F)F)ccc2c1O ; Cn1c(=O)c2c(ncn2CC(=O)O)n(C)c1=O ; Cc1cc(C)n(-c2ccc(C(=O)O)cc2)n1 ; O=C(O)C1CC(=O)N(Cc2cccc(Cl)c2)C1]

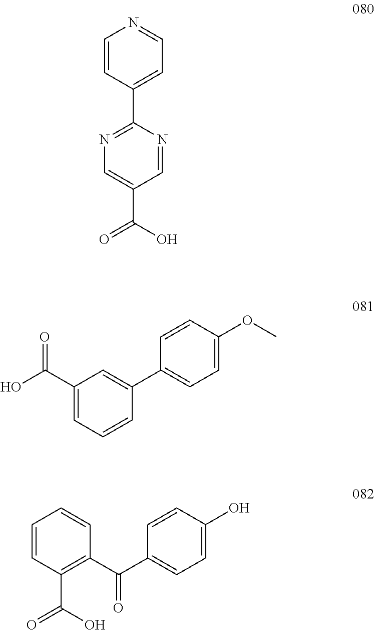 [SMILES: O=C(O)c1cnc(-c2ccncc2)nc1 ; COc1ccc(-c2cccc(C(=O)O)c2)cc1 ; O=C(O)c1ccccc1C(=O)c1ccc(O)cc1]

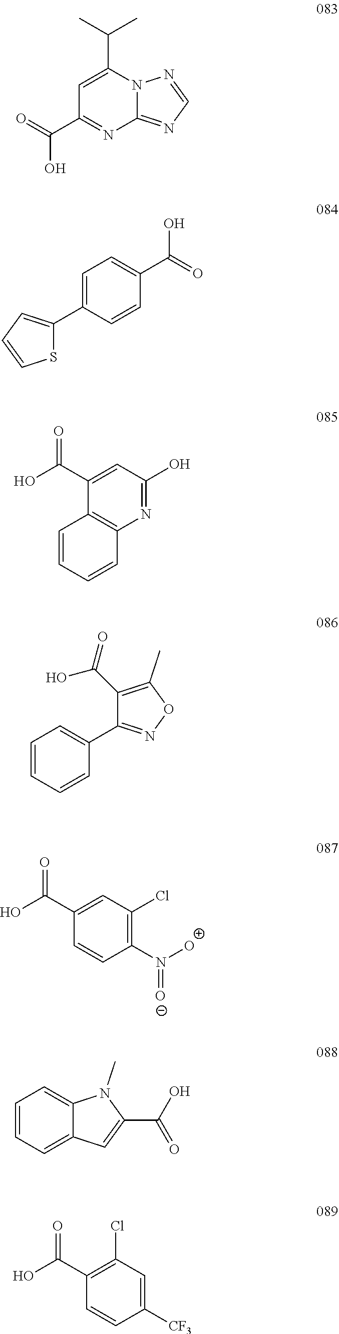

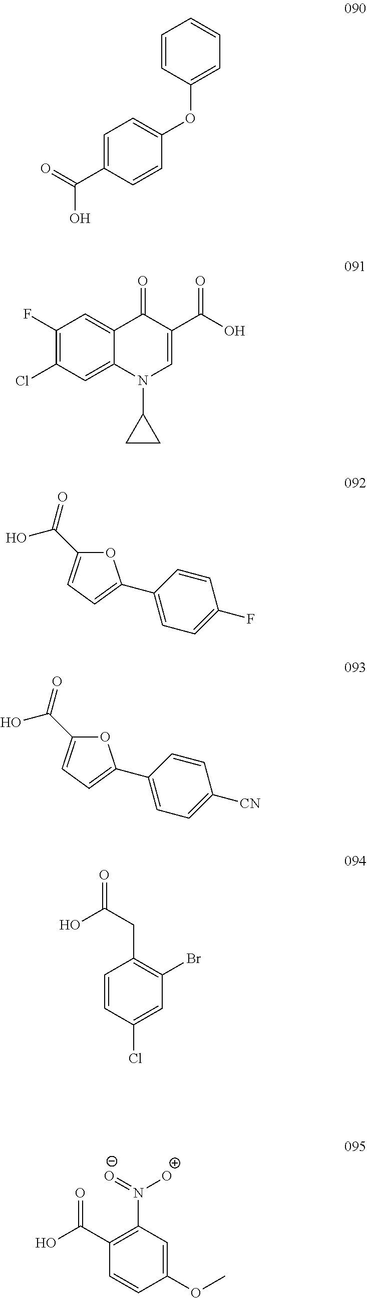

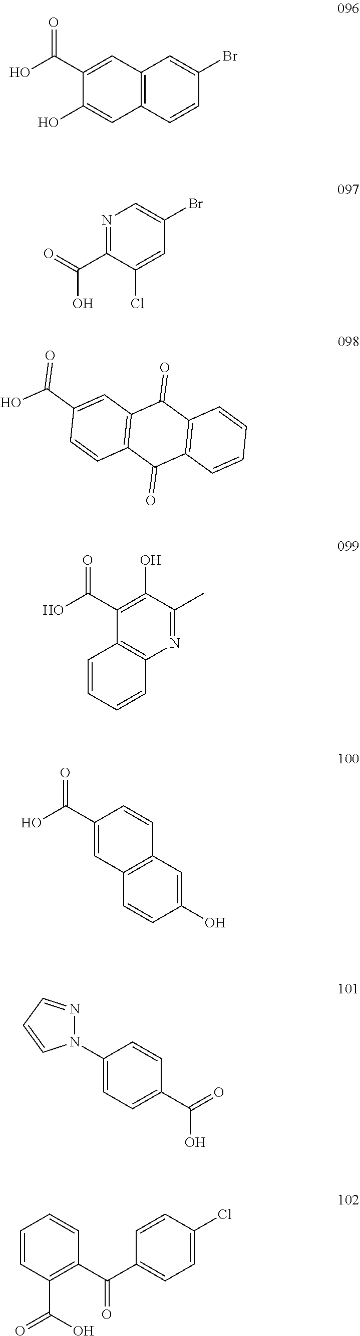

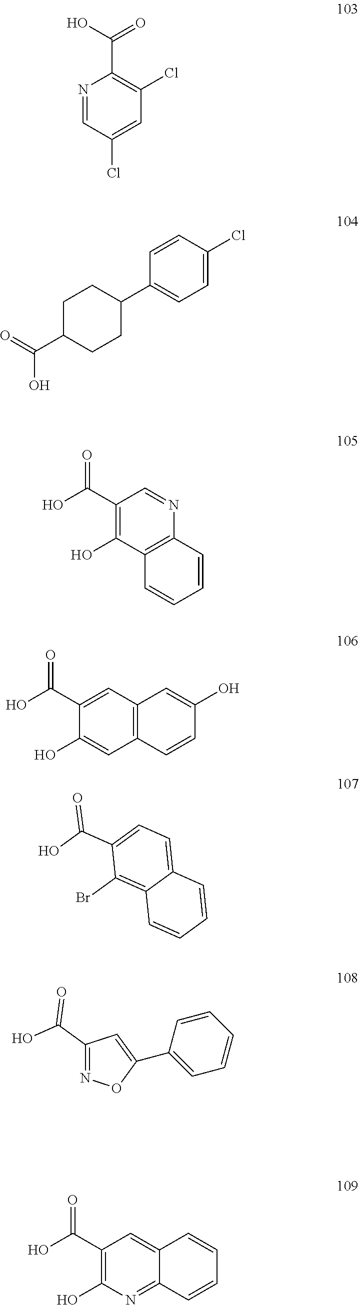

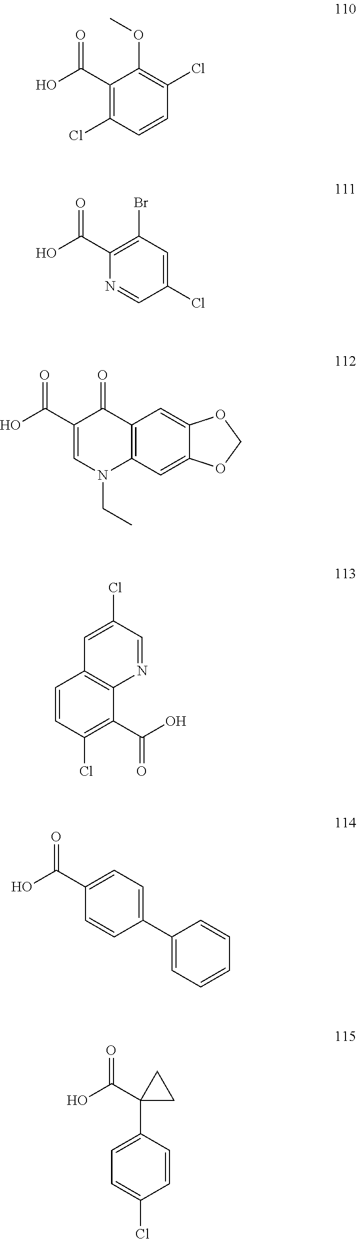 [SMILES: COc1c(Cl)ccc(Cl)c1C(=O)O ; O=C(O)c1ncc(Cl)cc1Br ; CCn1cc(C(=O)O)c(=O)c2cc3c(cc21)OCO3 ; O=C(O)c1c(Cl)ccc2cc(Cl)cnc12 ; O=C(O)c1ccc(-c2ccccc2)cc1 ; O=C(O)C1(c2ccc(Cl)cc2)CC1]

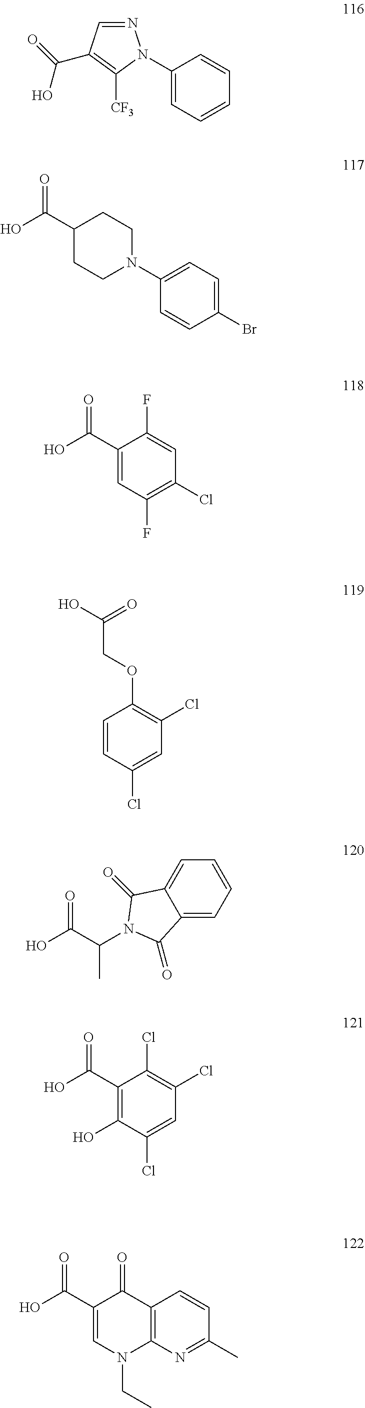

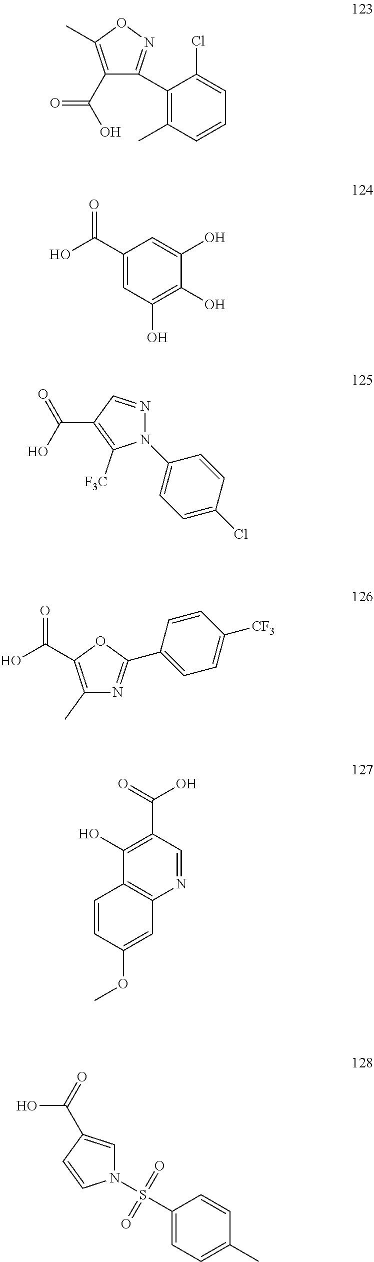 [SMILES: Cc1cccc(Cl)c1-c1noc(C)c1C(=O)O ; O=C(O)c1cc(O)c(O)c(O)c1 ; O=C(O)c1cnn(-c2ccc(Cl)cc2)c1C(F)(F)F ; Cc1nc(-c2ccc(C(F)(F)F)cc2)oc1C(=O)O ; COc1ccc2c(O)c(C(=O)O)cnc2c1 ; Cc1ccc(S(=O)(=O)n2ccc(C(=O)O)c2)cc1]

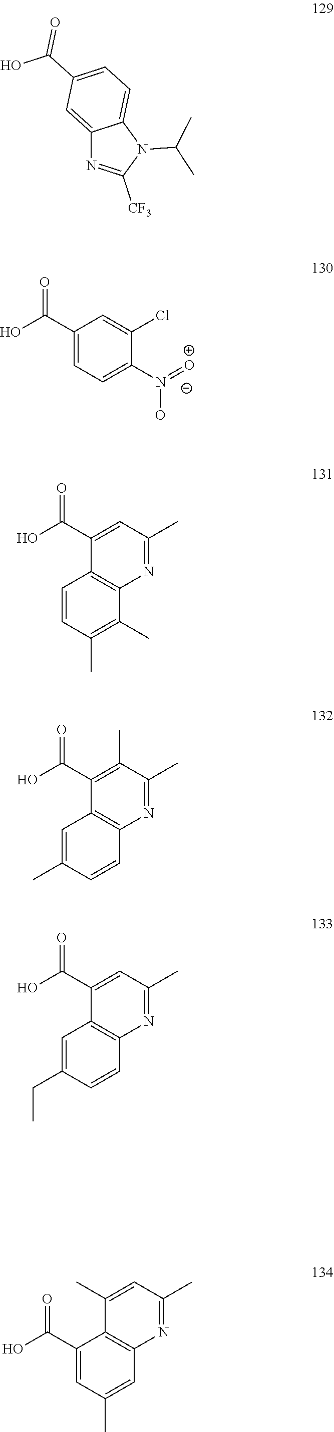

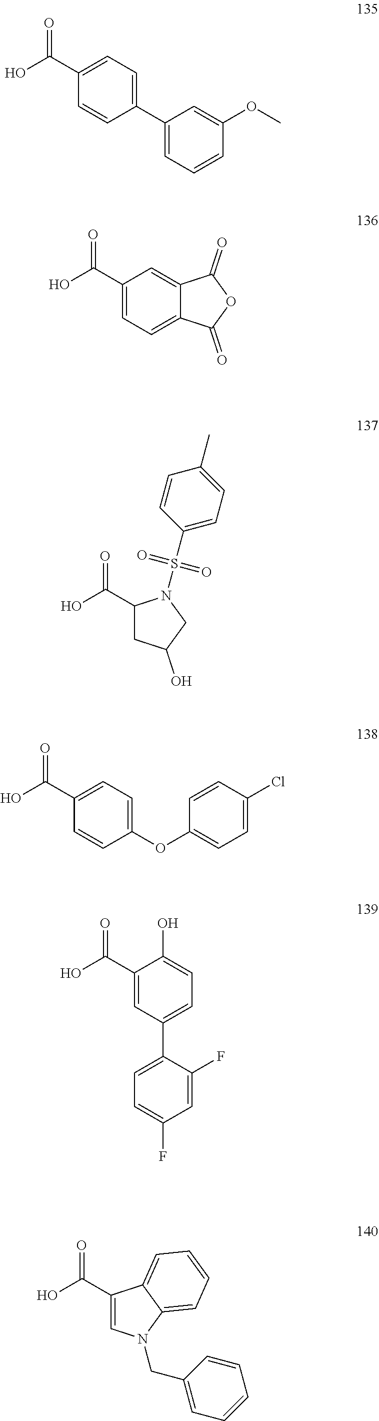 [SMILES: COc1cccc(-c2ccc(C(=O)O)cc2)c1 ; O=C(O)c1ccc2c(c1)C(=O)OC2=O ; Cc1ccc(S(=O)(=O)N2CC(O)CC2C(=O)O)cc1 ; O=C(O)c1ccc(Oc2ccc(Cl)cc2)cc1 ; O=C(O)c1cc(-c2ccc(F)cc2F)ccc1O ; O=C(O)c1cn(Cc2ccccc2)c2ccccc12]

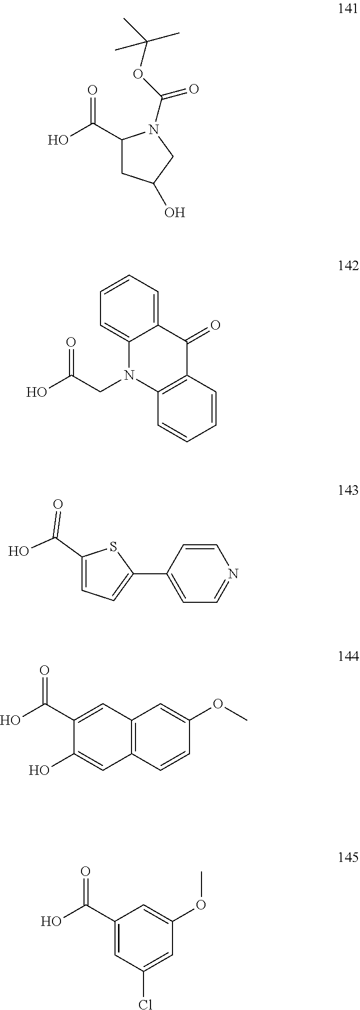 [SMILES: CC(C)(C)OC(=O)N1CC(O)CC1C(=O)O ; O=C(O)Cn1c2ccccc2c(=O)c2ccccc21 ; O=C(O)c1ccc(-c2ccncc2)s1 ; COc1ccc2cc(O)c(C(=O)O)cc2c1 ; COc1cc(Cl)cc(C(=O)O)c1]

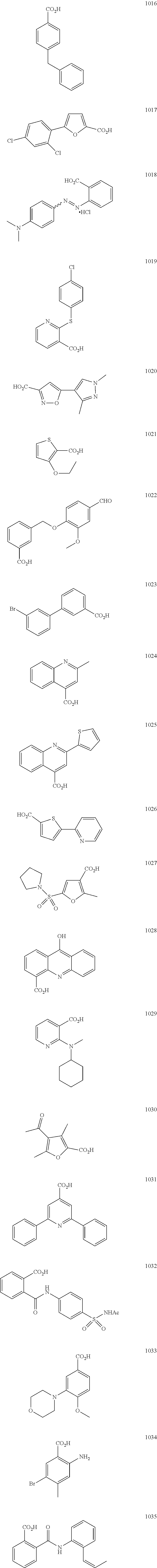

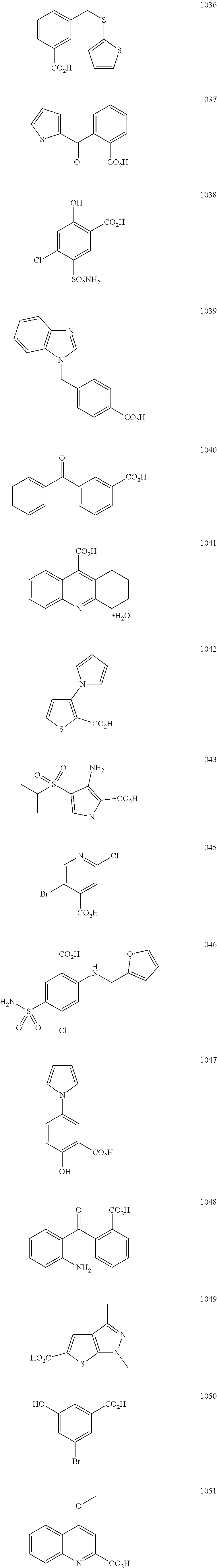

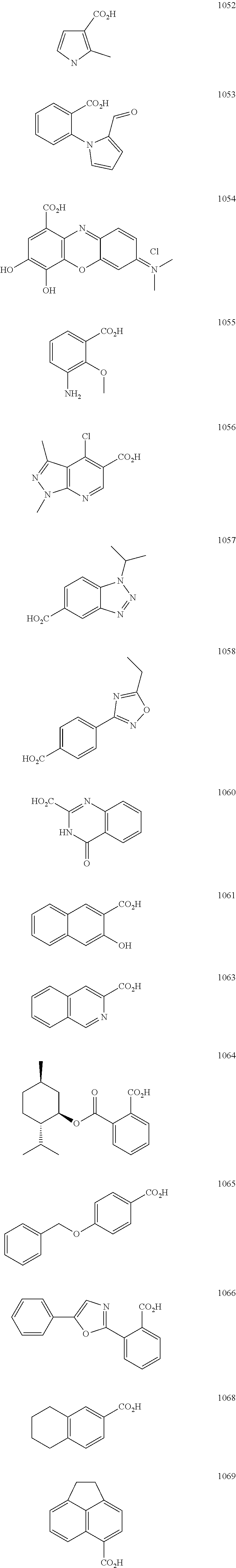

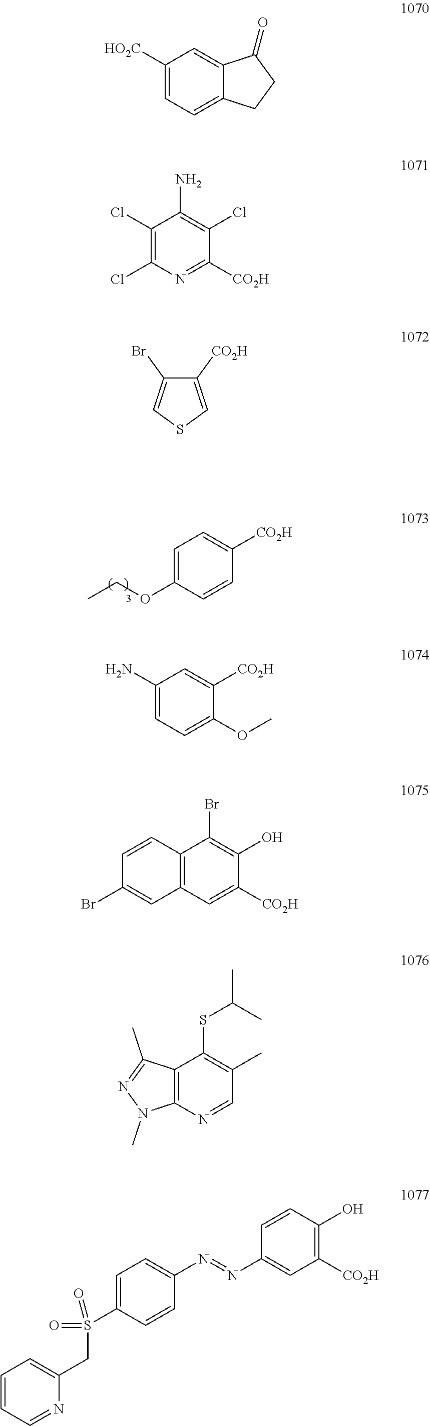

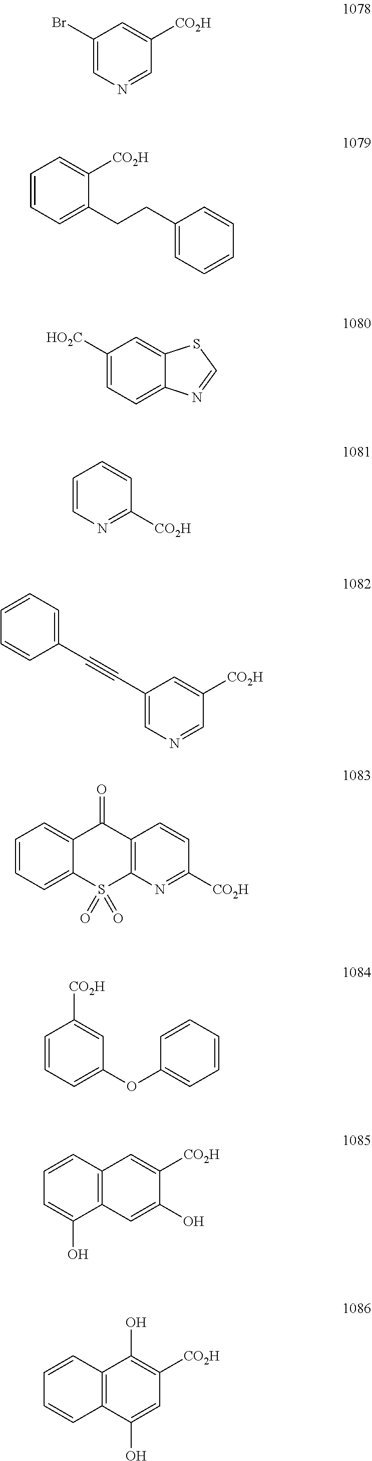

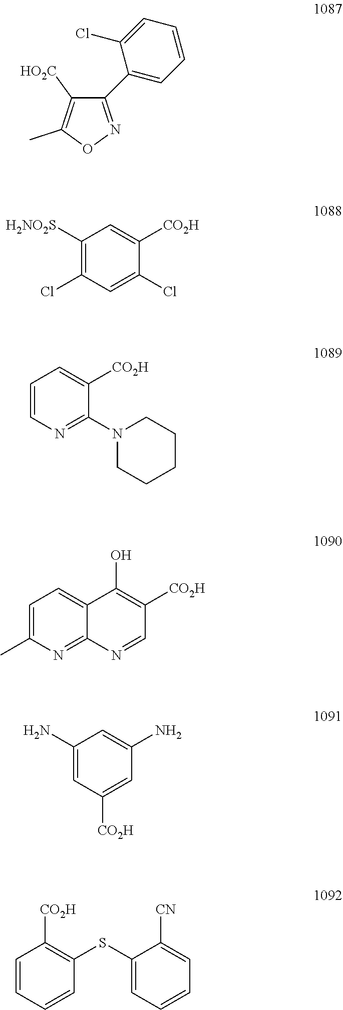 [SMILES: Cc1onc(-c2ccccc2Cl)c1C(=O)O ; NS(=O)(=O)c1cc(C(=O)O)c(Cl)cc1Cl ; O=C(O)c1cccnc1N1CCCCC1 ; Cc1ccc2c(O)c(C(=O)O)cnc2n1 ; Nc1cc(N)cc(C(=O)O)c1 ; N#Cc1ccccc1Sc1ccccc1C(=O)O]

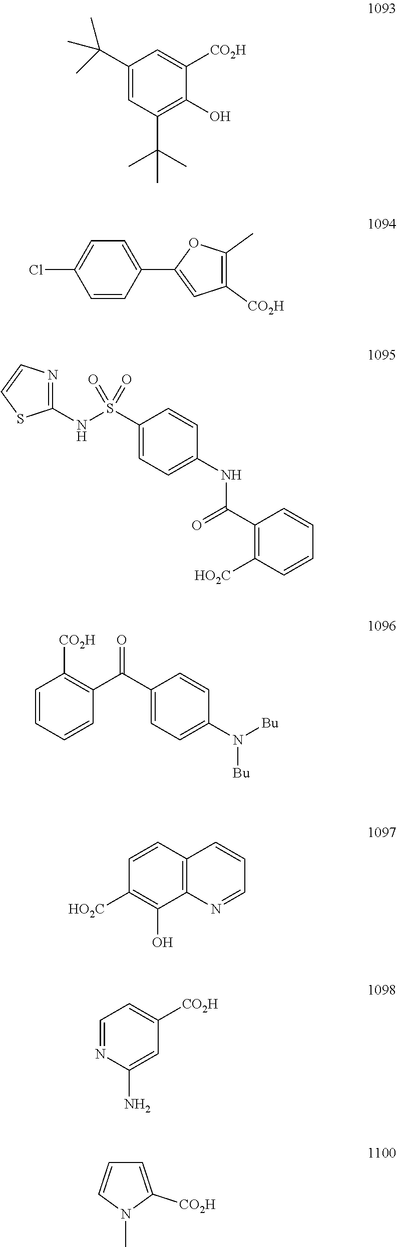

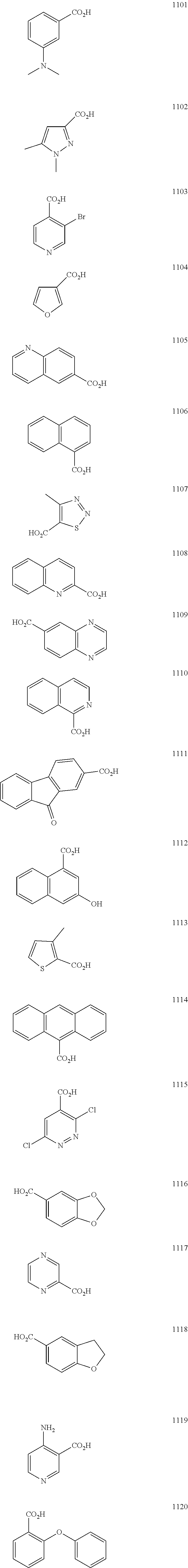

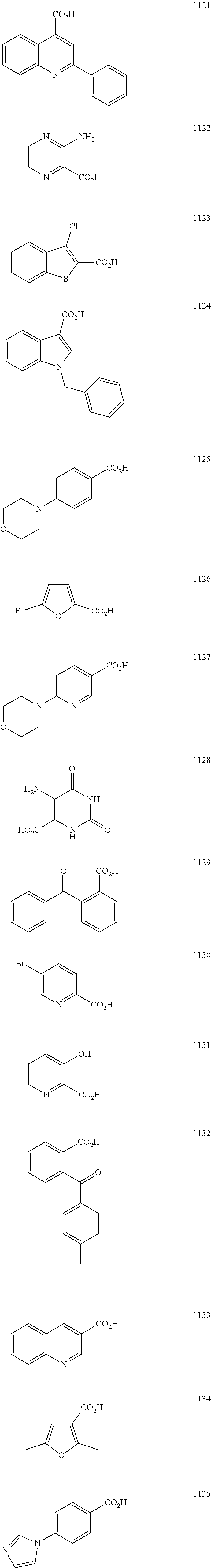

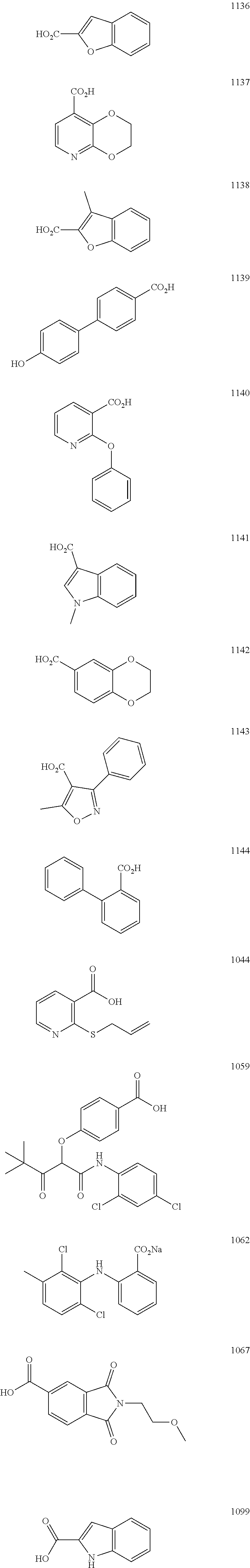

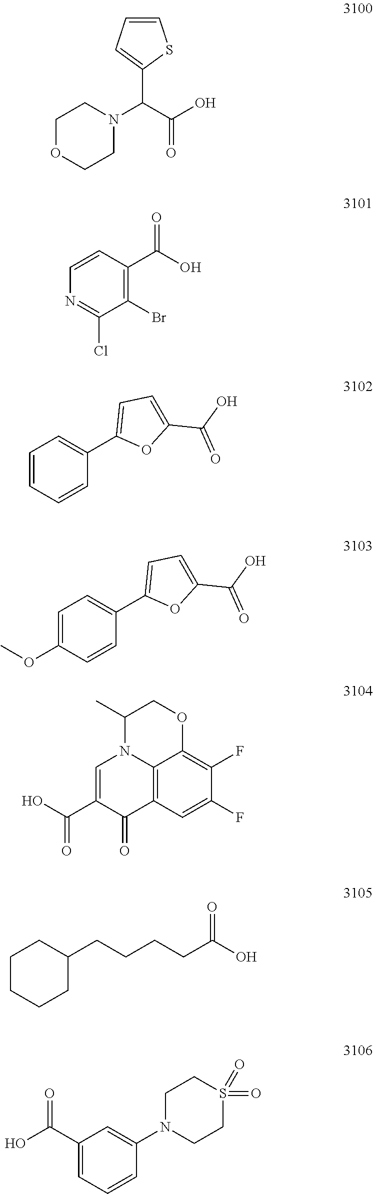

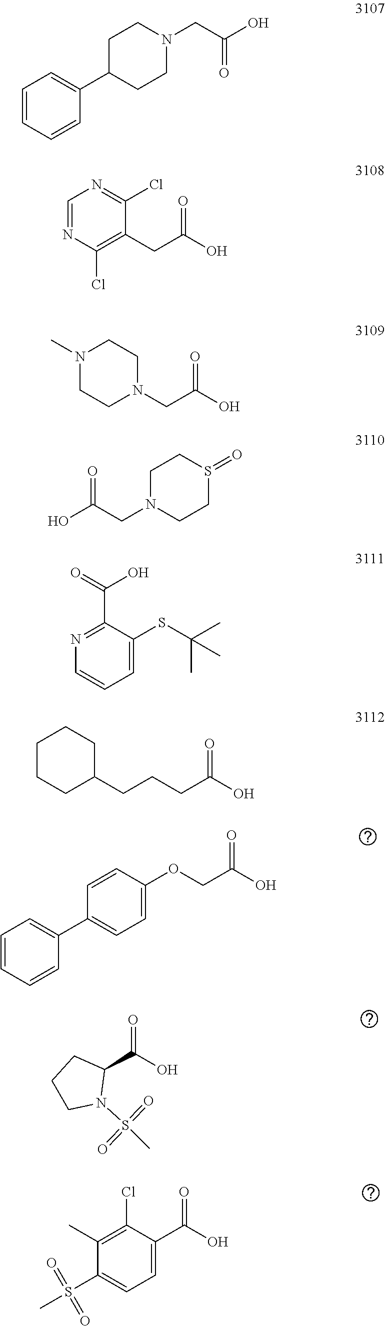

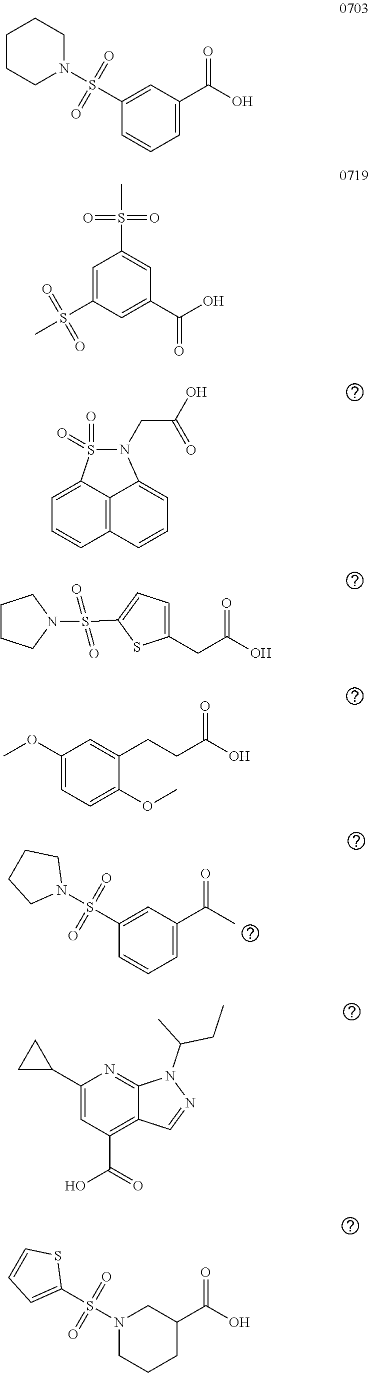

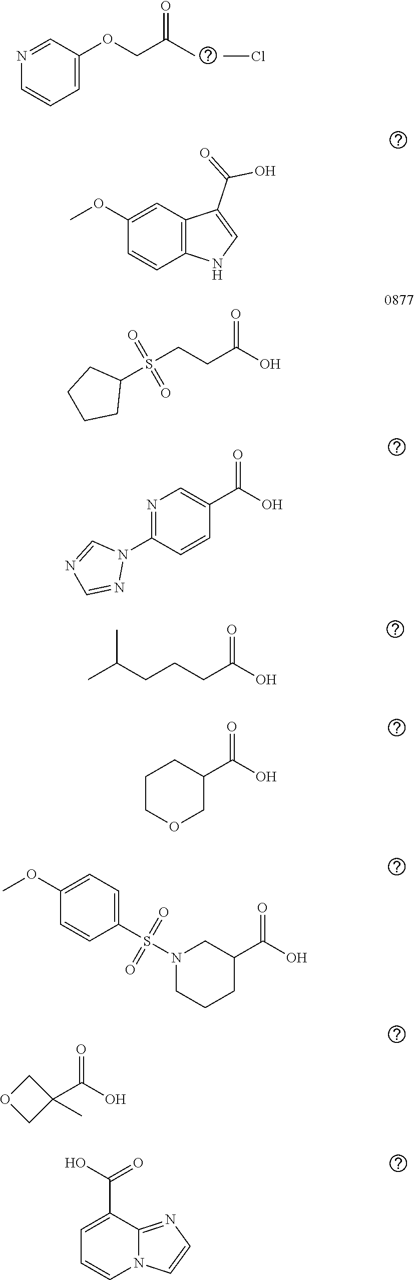

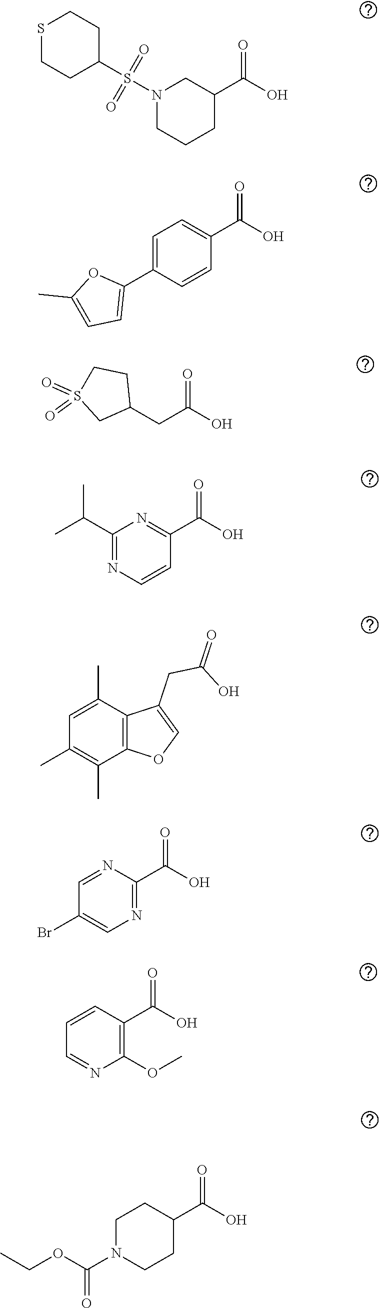

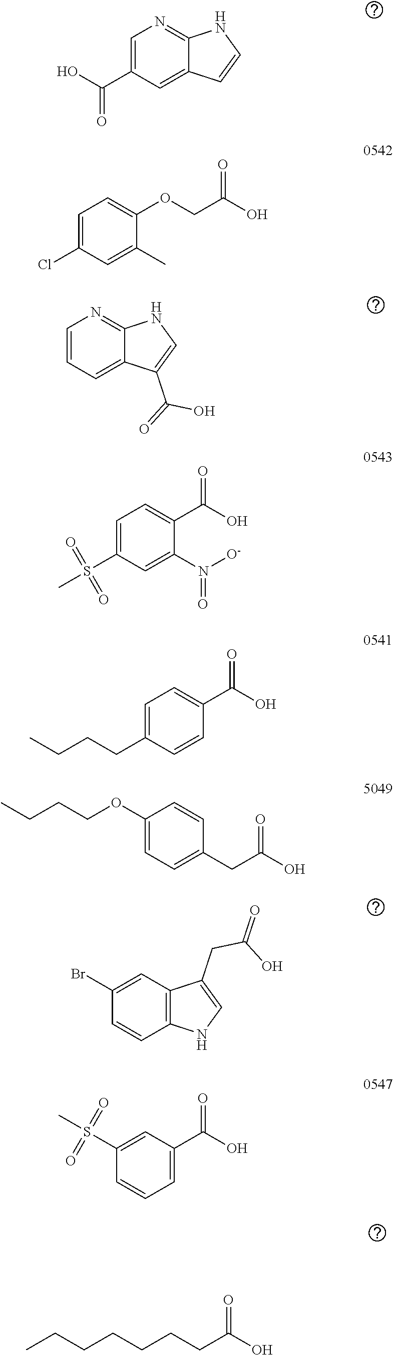

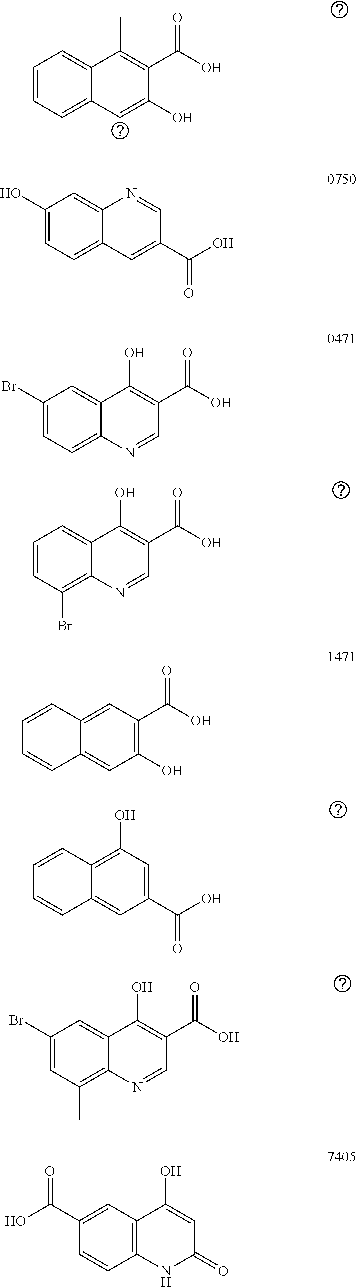

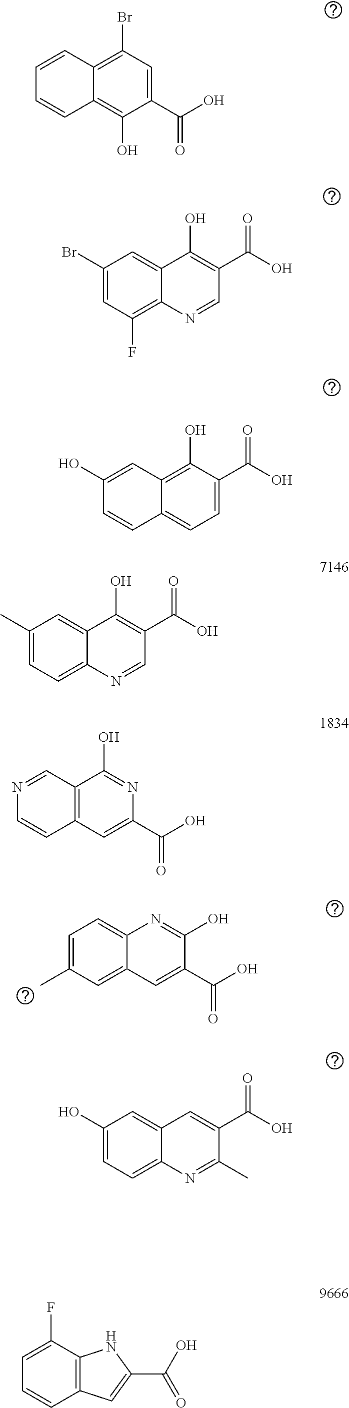

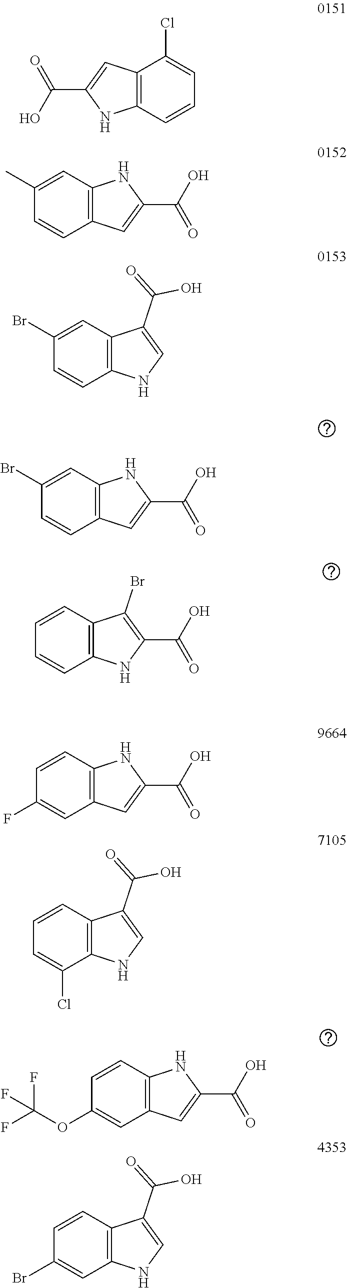

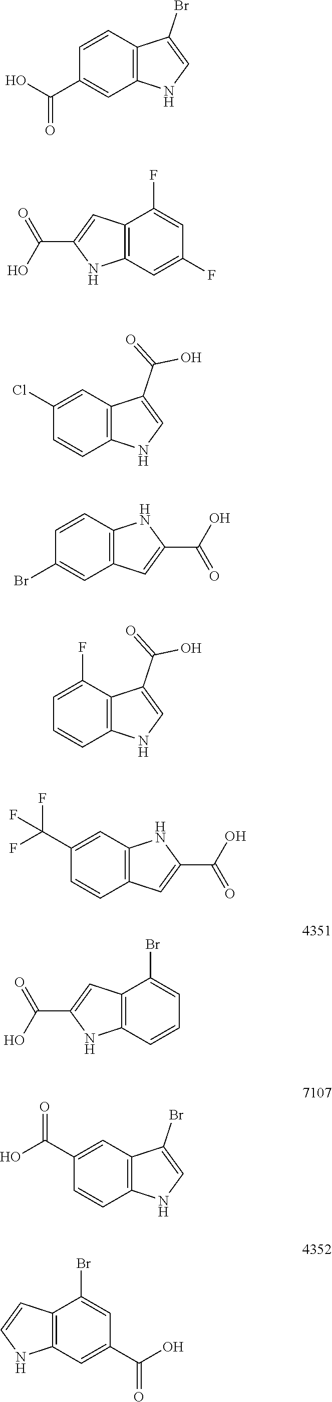

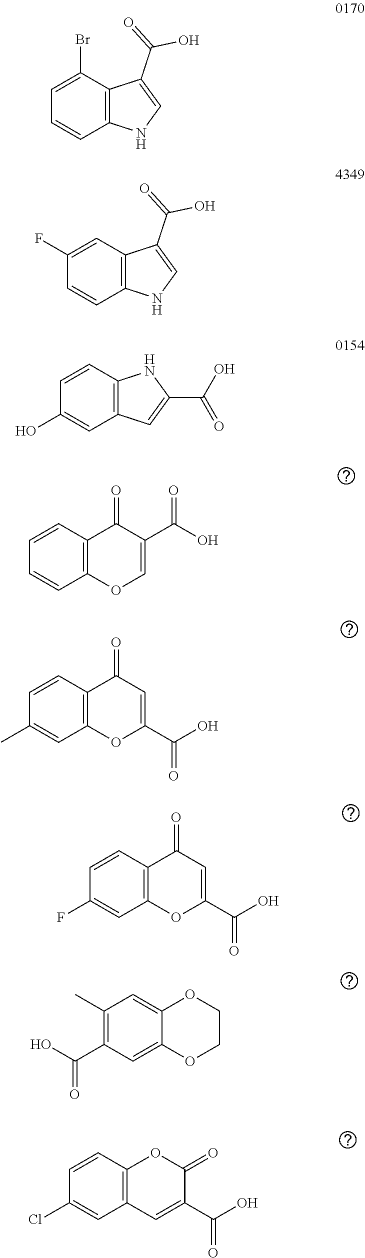

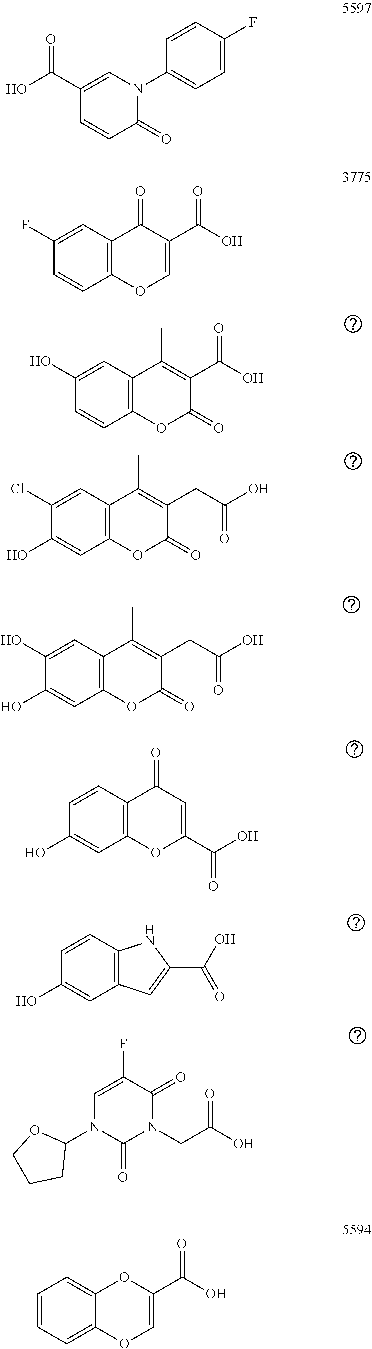

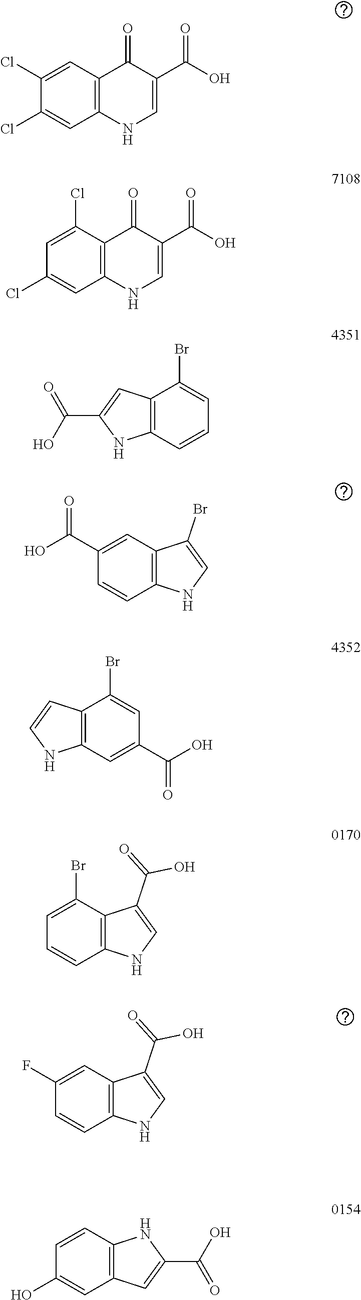

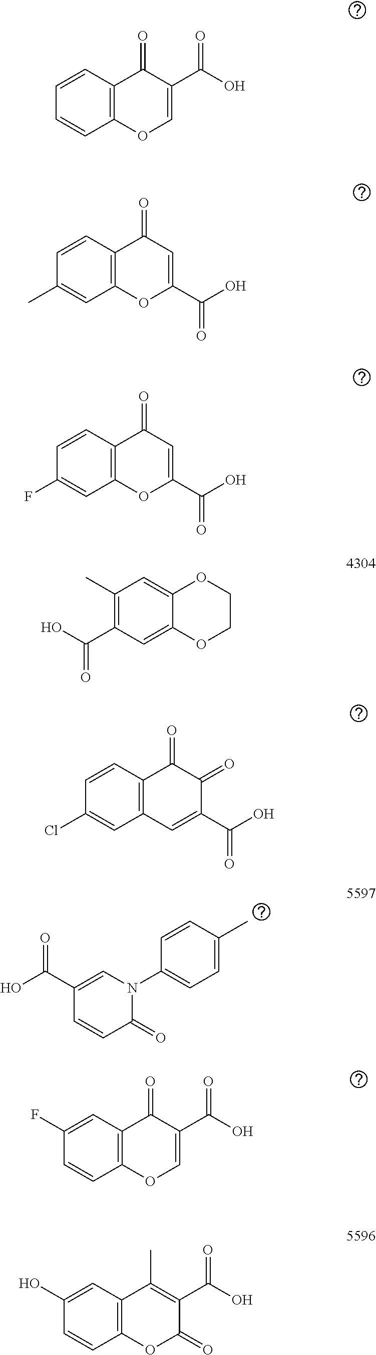

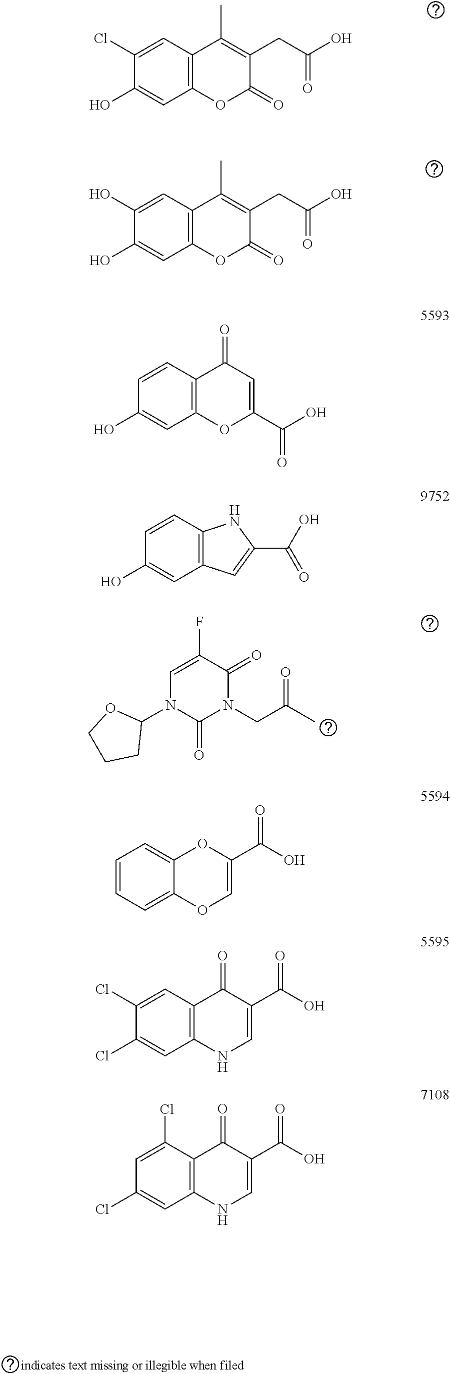

4. The compound of claim 1, wherein the compound of formula (I) is:

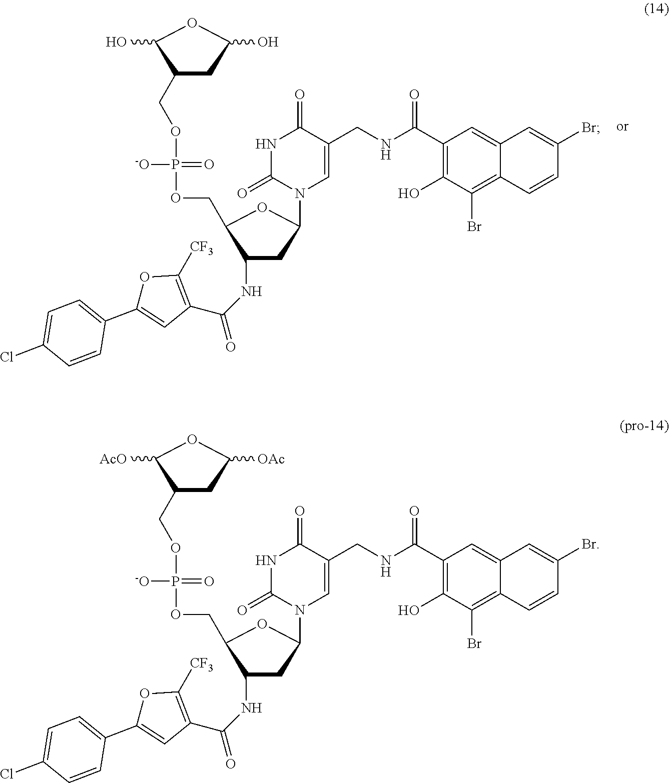

5. A method for irreversibly inhibiting a DNA polymerase in a subject, the method comprising administering to the subject a therapeutically effective amount of a compound of claim 1, or a pharmaceutically acceptable salt thereof.

6. The method of claim 5, wherein the DNA polymerase comprises a DNA repair enzyme.

7. The method of claim 6, wherein the DNA repair enzyme is DNA polymerase β.

8. The method of claim 5, wherein the compound inhibits the lyase and the polymerase activities of the DNA polymerase.

9. The method of claim 5, wherein the subject has cancer.

10. The method of claim 8, wherein inhibiting the DNA polymerase treats, inhibits, delays, or prevents the spread of the cancer in the subject.

11. The method of claim 9, further comprising treating, inhibiting, delaying, or preventing the spread of the cancer by inhibiting at least one cancer cell involved in one or more biological processes selected from the group consisting of cell migration, cell growth, cell adhesion, angiogenesis, cancer cell invasion, apoptosis, tumor formation, tumor progression, metastasis, degradation of the extracellular matrix, pericellular proteolysis, activation of plasminogen, and changes in the levels of an extracellular protease.

12. The method of claim 5, further comprising administering to the subject a DNA damaging agent.

13. The method of claim 11, wherein the DNA damaging agent is methyl methanesulfonate (MMS) or bleomycin.

14. The method of claim 11, wherein the DNA damaging agent is administered before or simultaneously with administration of the compound of Formula (I).

15. The method of claim 11, wherein the compound of formula (I) and the DNA damaging agent have a synergistic effect in treating a cancer.

16. A method for inhibiting a cancer cell, the method comprising contacting the cancer or noncancerous cell with a compound of claim 1, or a pharmaceutically acceptable salt thereof, in an amount effective to irreversibly inhibit a DNA polymerase.

17. A method for inducing a synthetic lethality in a breast cancer type 1 (BRCA1)-deficient cancer cell, the method comprising inhibiting DNA polymerase β by administering a compound of claim 1 to the cell.

18. A method for identifying one or more DNA polymerase inhibitors, the method comprising:
(a) contacting one or more candidate polymerase inhibitors with a DNA polymerase to form an inhibitor/polymerase solution;
(b) contacting the inhibitor/polymerase solution with a DNA template and deoxynucleotide triphosphate(s) (dNTP), and
(c) measuring polymerization using a fluorescently labeled DNA primer or a reagent that selectively binds to duplex DNA and fluoresces upon binding.

19. The method of claim 18, wherein the DNA template comprises a ternary DNA template.

20. The method of claim 18, wherein the ternary DNA template is fluorescently labeled at a 3′-terminus.

21. The method of claim 20, wherein the fluorescent label comprises TAMRA (carboxytetramethylrhodamine).

22. The method of claim 19, wherein the ternary DNA template is labeled at a 5′-terminus with a black hole quencher.

23. The method of claim 18, wherein the DNA polymerase comprises DNA polymerase β (pol β).

24. The method of claim 18, wherein an ability to prevent an increase in fluorescence indicates that the one or more candidate DNA polymerase inhibitors is a DNA polymerase inhibitor.

25. The method of claim 18, further comprising measuring a rate constant of fluorescence activity to determine a relative rate of DNA polymerase activity of the one or more candidate DNA polymerase inhibitors.

26. The method of claim 18, further comprising incubating the inhibitor/polymerase solution for a period of time before contacting it with the DNA template and deoxynucleotide triphosphate(s) (dNTP).

27. The method of claim 18, further comprising diluting the inhibitor/polymerase solution before contacting it with the DNA template and deoxynucleotide triphosphate(s) (dNTP).


SEQUENCE LISTING (11 sequences)

SEQ ID NO: 1 - DNA, 30 bp, Artificial sequence
  Synthetic
  misc_feature (16)..(16): N = A, C, T, or G
taatggctaacgcaanacgtaatgcagtct

SEQ ID NO: 2 - DNA, 30 bp, Artificial sequence
  Synthetic
agactgcattacgtattgcgttagccatta

SEQ ID NO: 3 - DNA, 29 bp, Artificial sequence
  Synthetic
tcaccctcgtacgactcttttttttttgc

SEQ ID NO: 4 - DNA, 29 bp, Artificial sequence
  Synthetic
gcaaaaaaaaaagagtcgtacgagggtga

SEQ ID NO: 5 - DNA, 13 bp, Artificial sequence
  Synthetic
tttttttaggttt

SEQ ID NO: 6 - DNA, 17 bp, Artificial sequence
  Synthetic
tcaccctcgtacgactc

SEQ ID NO: 7 - DNA, 22 bp, Artificial sequence
  Synthetic
tccaggactggtacgagggtga

SEQ ID NO: 8 - DNA, 34 bp, Artificial sequence
  Synthetic
accatgggacgtgctgactccactagatacactt

SEQ ID NO: 9 - DNA, 36 bp, Artificial sequence
  Synthetic
aggtgtatctagtggagtctcagcacgtcccatggt

SEQ ID NO: 10 - DNA, 29 bp, Artificial sequence
  Synthetic
taatggctaacgcttccgtaatgcagtct

SEQ ID NO: 11 - DNA, 30 bp, Artificial sequence
  Synthetic
agactgcattacggaaagcgttagccatta